(19) United States
(12) Patent Application Publication
(10) Pub. No.: US 2025/0281653 A1
(43) Pub. Date: Sep. 11, 2025

(54) METHODS FOR TREATING PROSTATE CANCER WITH RADIOPHARMACEUTICALS

(71) Applicant: ARTBIO, Inc., Cambridge, MA (US)

(72) Inventors: Victoria Louise JARDINE, Denham (GB); Aurélie BOUZELMAT, London (GB)

(21) Appl. No.: 19/210,753

(22) Filed: May 16, 2025

Related U.S. Application Data
(63) Continuation of application No. PCT/US2025/018987, filed on Mar. 7, 2025.
(60) Provisional application No. 63/563,214, filed on Mar. 8, 2024.
(60) Provisional application No. 63/563,921, filed on Mar. 11, 2024.
(60) Provisional application No. 63/647,921, filed on May 15, 2024.
(60) Provisional application No. 63/705,945, filed on Oct. 10, 2024.

(51) Int. Cl. A61K 51/04; A61K 31/519

(52) U.S. Cl. CPC A61K 51/0497; A61K 31/519; A61K 51/0406; A61K 2121/00

(57) ABSTRACT

Provided herein are methods of treating prostate cancer using radiopharmaceuticals. The radiopharmaceuticals compositions can comprise a prostate specific membrane antigen (PSMA) targeting moiety, a metal chelator, and a radionuclide chelated to the metal chelator.

### CROSS-REFERENCE TO RELATED APPLICATIONS

This application is a continuation of International Application No. PCT/US2025/018987, filed Mar. 7, 2025, which claims the benefit of U.S. Provisional Application No. 63/563,214 filed Mar. 8, 2024, U.S. Provisional Application No. 63/563,921 filed Mar. 11, 2024, U.S. Provisional Application No. 63/647,921 filed May 15, 2024, and U.S. Provisional Application No. 63/705,945 filed Oct. 10, 2024, each of which is incorporated by reference herein in their entirety.

### BACKGROUND

Prostate-specific membrane antigen (PSMA) is a transmembrane glycoprotein found in more than 90% of prostate cancer cells at levels 100-1000 greater than normal cells. Targeted cancer therapy that is highly sensitive and specific to PSMA expressing tissues has the potential to induce tumor cell death while sparing normal tissues. PSMA-targeted radiopharmaceutical therapies are being developed due to the sensitivity of prostate cancer to radiation. The PSMA targeted radiopharmaceutical ²²⁵Ac-PSMA-617 was reported to have the potential to achieve a complete prostate cancer remission; however, dose limiting toxicities resulted from undesired salivary gland uptake (Kratochwil et al. J. Nucl. Med. 2017; 58:1624-1631). Salivary glands physiologically express PSMA, and targeted therapy accumulation in the salivary glands can be attributed to a combination of both PSMA specific and non-specific uptake mechanisms.

### SUMMARY

Undesired salivary gland uptake of PSMA-targeted radiotherapies can damage the tissue leading to development of xerostomia, a dry mouth, as a critical side effect. Salivary gland damage leading to xerostomia can depend on several factions a) the type of radiation therapy (external beam radiation therapy vs radiotherapeutic agent) b) the radionuclide used (alpha vs beta emission, properties of radionuclide rate of decay) and c) total salivary gland uptake of the PSMA-targeted radiotherapy. Factors b) and c) of these can influence how the administered radioactivity in MBq or GBq relates to the absorbed dose to the salivary glands in Gy, or for alpha emitters in Sv as a relative biological effect (RBE) also needs to be applied to account for the greater potency. For external beam radiation therapy (EBRT), minimal functional impairment occurs at mean doses of <10-15 Gy. Increasing doses in the range of 20-30Gy lead to progressive deterioration. (Gupta et al. Radiation Oncology, 2015; 10:67). For radiotherapeutic agents (e.g., with ¹⁷⁷Lu or alpha emitters) the full dose/toxicity profile for the salivary glands is still being established, however it is noted as a common or dose limiting toxicity in all small molecule-based agents.

Administration of 7.4 GBq (200 mCi) of ¹⁷⁷Lu-PSMA-617 once every 6 weeks (up to 6 cycles) is estimated to provide 4.5 Gy per cycle (i.e., 27 Gy over 6 cycles) of radiation to the salivary glands. Dry mouth (i.e., xerostomia) occurred in 38.8% of the population of the phase 3 VISION study; all grade 1 or 2 severity (Sartor et al. NEJM 2021; 385; 12 1091-1103).

Multiple early phase/compassionate use studies with ²²⁵Ac-PSMA-617 have been published, typically reporting xerostomia as a key toxicity occurring in most/all patients. Severe xerostomia was identified as a dose-limiting above 100 kBq/kg, i.e., approximate 7 MBq for a 70 kg male patient (Kratochwil et al. J. Nucl. Med. 2017; 58:1624-1631). Salivary gland absorbed dose estimate for 1 MBq of ²¹⁵Ac-PSMA-617 was 2.3 Sv (using a relative biological effect (RBE) of 5), for a single dose of 7.4 MBq this equates to 17.24 Sv. Typically 4 to 6 doses given in 6 to 8 week cycles are proposed; 4 cycles would equate to 68.96 Sv and 6 cycles to 103.44 Sv.

Accordingly, there remains a need for the development of PSMA-targeted radiotherapies with reduced or eliminated uptake into salivary glands.

In one aspect, provided herein is a method of treating a cancer in a subject in need thereof, comprising administering to the subject a radiopharmaceutical conjugate, wherein the radiopharmaceutical conjugate comprises,
(a) a metal chelator;
(b) a target-binding moiety; and
(c) a radionuclide bound to the metal chelator, wherein the radionuclide is ²¹²Pb; wherein the radiopharmaceutical conjugate is administered to the subject about every 1 to 7 weeks. In some embodiments, the radiopharmaceutical conjugate comprises a structure of Formula (I):

TL-L-R^{M} Formula (I)
or a pharmaceutically acceptable salt thereof, wherein:
TL is a PSMA targeting ligand comprising a urea;
L is a bivalent linking moiety; and
R^{M} is a metal chelator.

In some embodiments, the cancer is a prostate cancer.

In one aspect, provided herein is a method of treating a prostate cancer in a subject in need thereof, comprising administering to the subject a radiopharmaceutical conjugate, wherein the radiopharmaceutical conjugate comprises,
(a) a compound having the structure:

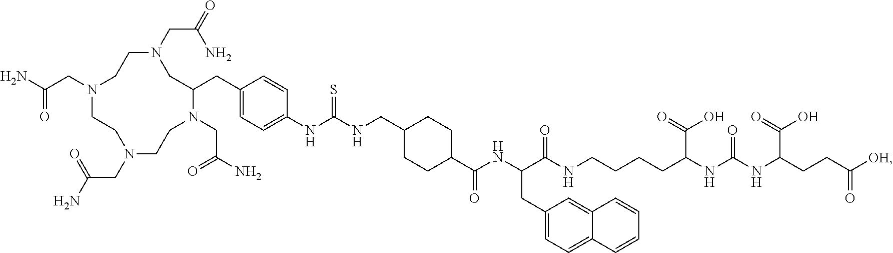 [SMILES: NC(=O)CN1CCN(CC(N)=O)CCN(CC(N)=O)C(Cc2ccc(NC(=S)NCC3CCC(C(=O)NC(Cc4ccc5ccccc5c4)C(=O)NCCCCC(NC(=O)NC(CCC(=O)O)C(=O)O)C(=O)O)CC3)cc2)CN(CC(N)=O)CC1]

or a pharmaceutically acceptable salt thereof; and
(b) a radionuclide bound to the metal chelator, wherein the radionuclide is ²¹²Pb; wherein the radiopharmaceutical conjugate is administered to the subject in an amount between 50 kBq to 500 MBq per dose. In some embodiments, the radiopharmaceutical conjugate is administered in an amount of about 100 kBq to 500 MBq per dose. In some embodiments, the radiopharmaceutical conjugate is administered in an amount of about 1 MBq to 500 MBq per dose. In some embodiments, the radiopharmaceutical conjugate is administered in an amount of about 10 MBq to 500 MBq per dose. In some embodiments, the radiopharmaceutical conjugate is administered in an amount of about 50 MBq to 500 MBq per dose. In some embodiments, the radiopharmaceutical conjugate is administered in an amount of about 10 MBq per dose. In some embodiments, the radiopharmaceutical conjugate is administered in an amount of about 100 MBq per dose. In some embodiments, the radiopharmaceutical conjugate is administered in an amount of about 75 MBq to about 300 MBq per dose. In some embodiments, the radiopharmaceutical conjugate is administered in an amount of about 75 MBq to about 300 MBq per dose. In some embodiments, the radiopharmaceutical conjugate is administered in an amount of about 70 MBq per dose about 75 MBq per dose, about 80 MBq per dose, about 85 MBq per dose, about 90 MBq per dose, about 95 MBq per dose, about 100 MBq per dose, about 105 MBq per dose, about 110 MBq per dose, about 115 MBq per dose, about 120 MBq per dose, about 125 MBq per dose, about 130 MBq per dose, about 140 MBq per dose, about 145 MBq per dose, about 150 MBq per dose, about 155 MBq per dose, about 160 MBq per dose, about 165 MBq per dose, about 170 MBq per dose, about 175 MBq per dose, about 180 MBq per dose, about 185 MBq per dose, about 190 MBq per dose, about 195 MBq per dose, about 200 MBq per dose, about 205 MBq per dose, about 210 MBq per dose, about 215 MBq per dose, about 220 MBq per dose, about 225 MBq per dose, about 230 MBq per dose, about 235 MBq per dose, about 240 MBq per dose, about 245 MBq per dose, about 250 MBq per dose, about 255 MBq per dose, about 260 MBq per dose, about 265 MBq per dose, about 270 MBq per dose, about 275 MBq per dose, about 280 MBq per dose, about 285 MBq per dose, about 290 MBq per dose, about 295 MBq per dose, or about 300 MBq per dose. In some embodiments, the radiopharmaceutical conjugate is administered in an amount of about 75 MBq per dose, about 100 MBq per dose, about 125 MBq per dose, about 150 MBq per dose, about 175 MBq per dose, about 200 MBq per dose, about 225 MBq per dose, about 250 MBq per dose, about 275 MBq per dose, or about 300 MBq per dose. In some embodiments, the radiopharmaceutical conjugate is administered in an amount of about 75 MBq per dose, about 100 MBq per dose, about 150 MBq per dose, about 200 MBq per dose, about 250 MBq per dose, or about 300 MBq per dose. In some embodiments, the radiopharmaceutical conjugate is administered in an amount of about 75 MBq per dose. In some embodiments, the radiopharmaceutical conjugate is administered in an amount of about 100 MBq per dose. In some embodiments, the radiopharmaceutical conjugate is administered in an amount of about 125 MBq per dose. In some embodiments, the radiopharmaceutical conjugate is administered in an amount of about 150 MBq per dose. In some embodiments, the radiopharmaceutical conjugate is administered in an amount of about 175 MBq per dose. In some embodiments, the radiopharmaceutical conjugate is administered in an amount of about 200 MBq per dose. In some embodiments, the radiopharmaceutical conjugate is administered in an amount of about 225 MBq per dose. In some embodiments, the radiopharmaceutical conjugate is administered in an amount of about 250 MBq per dose. In some embodiments, the radiopharmaceutical conjugate is administered in an amount of about 275 MBq per dose. In some embodiments, the radiopharmaceutical conjugate is administered in an amount of about 300 MBq per dose.

In one aspect, provided herein is a method of treating a prostate cancer in a subject in need thereof, comprising administering to the subject a radiopharmaceutical conjugate, wherein the radiopharmaceutical conjugate comprises,
(a) a compound having the structure:

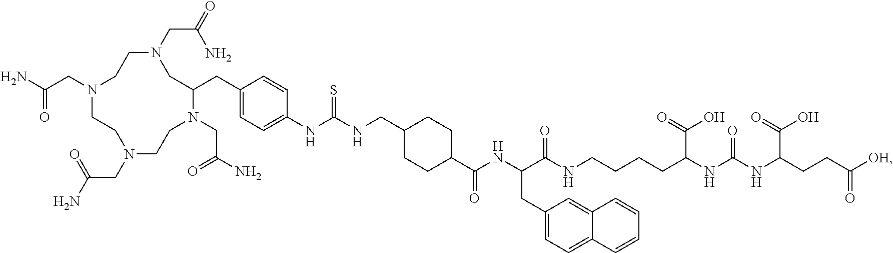 [SMILES: NC(=O)CN1CCN(CC(N)=O)CCN(CC(N)=O)C(Cc2ccc(NC(=S)NCC3CCC(C(=O)NC(Cc4ccc5ccccc5c4)C(=O)NCCCCC(NC(=O)NC(CCC(=O)O)C(=O)O)C(=O)O)CC3)cc2)CN(CC(N)=O)CC1]

or a pharmaceutically acceptable salt thereof; and
(b) a radionuclide bound to the metal chelator, wherein the radionuclide is ²¹²Pb; wherein the radiopharmaceutical conjugate is administered without an agent for treating xerostomia.

In one aspect, provided herein is a method of treating a prostate cancer in a subject in need thereof, comprising administering to the subject a radiopharmaceutical conjugate, wherein the radiopharmaceutical conjugate comprises,
(a) a compound having the structure:

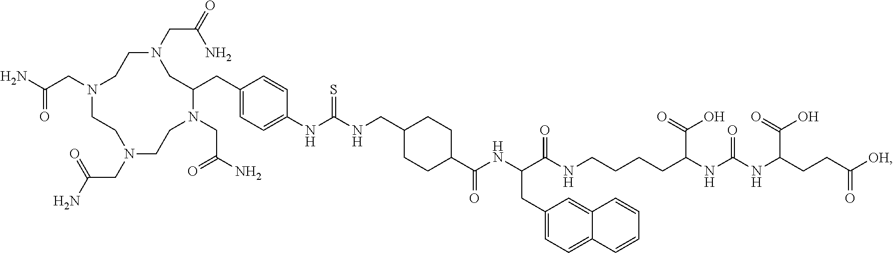 [SMILES: NC(=O)CN1CCN(CC(N)=O)CCN(CC(N)=O)C(Cc2ccc(NC(=S)NCC3CCC(C(=O)NC(Cc4ccc5ccccc5c4)C(=O)NCCCCC(NC(=O)NC(CCC(=O)O)C(=O)O)C(=O)O)CC3)cc2)CN(CC(N)=O)CC1]

or a pharmaceutically acceptable salt thereof; and
(b) a radionuclide bound to the metal chelator, wherein the radionuclide is ²¹²Pb; wherein the subject has previously received an anti-cancer agent before the administering of the radiopharmaceutical conjugate

In one aspect, provided herein is a method of treating a prostate cancer in a subject in need thereof, comprising administering to the subject a radiopharmaceutical conjugate, wherein the radiopharmaceutical conjugate comprises,
(a) a compound having the structure:

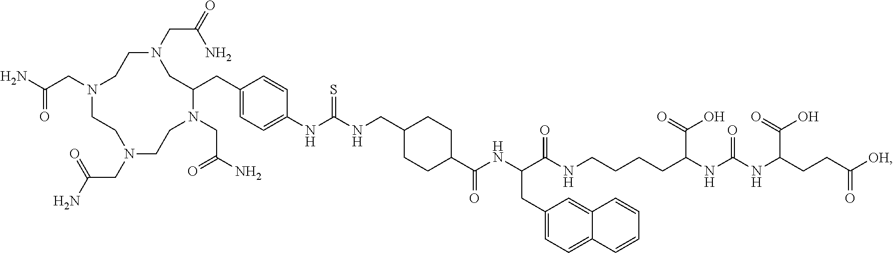 [SMILES: NC(=O)CN1CCN(CC(N)=O)CCN(CC(N)=O)C(Cc2ccc(NC(=S)NCC3CCC(C(=O)NC(Cc4ccc5ccccc5c4)C(=O)NCCCCC(NC(=O)NC(CCC(=O)O)C(=O)O)C(=O)O)CC3)cc2)CN(CC(N)=O)CC1]

or a pharmaceutically acceptable salt thereof; and
(b) a radionuclide bound to the metal chelator, wherein the radionuclide is ²¹²Pb; wherein the radiopharmaceutical conjugate is administered in combination with a second anti-cancer agent.

In one aspect, provided herein is a method of treating a prostate cancer in a subject in need thereof, comprising administering
(a) an imaging radiopharmaceutical agent comprising a first radionuclide; and
(b) a radiopharmaceutical conjugate comprising:
   (i) a compound having the structure:

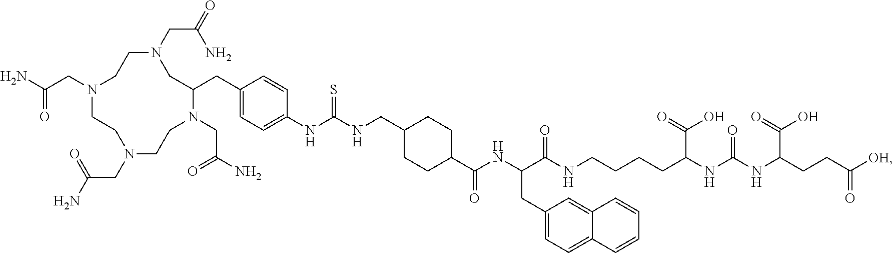 [SMILES: NC(=O)CN1CCN(CC(N)=O)CCN(CC(N)=O)C(Cc2ccc(NC(=S)NCC3CCC(C(=O)NC(Cc4ccc5ccccc5c4)C(=O)NCCCCC(NC(=O)NC(CCC(=O)O)C(=O)O)C(=O)O)CC3)cc2)CN(CC(N)=O)CC1]

or a pharmaceutically acceptable salt thereof; and
(ii) a second radionuclide bound to the metal chelator, wherein the second radionuclide is ²¹²Pb;
   wherein the imaging radiopharmaceutical agent is administered to the patient prior to the administering of the radiopharmaceutical conjugate.

In one aspect, provided herein is a liquid radiopharmaceutical composition comprising
(a) a compound having the structure:

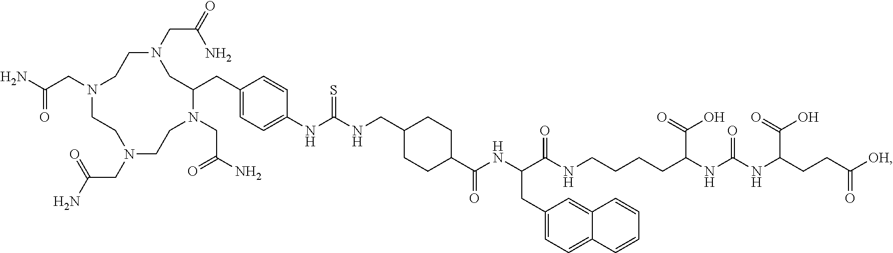 [SMILES: NC(=O)CN1CCN(CC(N)=O)CCN(CC(N)=O)C(Cc2ccc(NC(=S)NCC3CCC(C(=O)NC(Cc4ccc5ccccc5c4)C(=O)NCCCCC(NC(=O)NC(CCC(=O)O)C(=O)O)C(=O)O)CC3)cc2)CN(CC(N)=O)CC1]

or a pharmaceutically acceptable salt thereof; and
(b) a radionuclide bound to the metal chelator, wherein the radionuclide is ²¹²Pb,
   wherein the liquid radiopharmaceutical composition has a radioactivity of about 75 MBq to about 300 MBq.

### INCORPORATION BY REFERENCE

All publications, patents, and patent applications mentioned in this specification are herein incorporated by reference for the specific purposes identified herein.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing (also “figure” and “FIG.” herein), of which:

FIG. 1 illustrates timepoints of planar and SPECT imaging, whole-body probe measurements and blood sample draws.

FIG. 2 illustrates the decay chain of ²¹²Pb including energy of X-rays and Gamma emissions. See From: Kvassheim, M., Revheim, ME. R. & Stokke, C. Quantitative SPECT/CT imaging of lead-212: a phantom study. EJNMMI Phys 9, 52 (2022).

FIG. 3 illustrates the energy spectra indicating ²¹²Bi and ²¹²Pb windows.

FIG. 4A-4B illustrates the SPECT/CT imaging in participant NO-01-001: normal tissue. SPECT imaging following ²¹²Pb-Compound 1 administration illustrating normal tissue for participant NO-01-001 on Day 0 (1 hour timepoint): FIG. 4A illustrates whole body MIP (anterior view); and FIG. 4B illustrates axial section through the cranium at the level of the parotid glands. ²¹²Pb-Compound 1 uptake could not be visualized in the parotid glands.

FIG. 5A-5D illustrates the planar gamma imaging in participant N0-01-002: normal tissue. Planar gamma imaging following ²¹²Pb-Compound 1 administration illustrating normal tissue for participant NO-01-002 on Day 0 (FIG. 5A and FIG. 5B illustrate anterior and posterior views, respectively, at the 1 hour timepoint post injection) and Day 1 (FIG. 5C and FIG. 5D illustrate anterior and posterior views respectively at the 16 hour timepoint post injection).

FIG. 6A-6F illustrates the SPECT/CT imaging in participant N0-01-003; abdominal retrocaval lymph node. Paired SPECT (summed) (FIG. 6A, FIG. 6C, and FIG. 6E) and non-contrast enhanced CT scans (FIG. 6B, FIG. 6D, and FIG. 6F) from the SPECT/CT for participant NO-01-003 on Day 0 (1 hour timepoint): FIG. 6A and FIG. 6B illustrate axial section; FIG. 6C and FIG. 6D illustrate sagittal section;

FIG. 6E and FIG. 6F illustrate coronal section. The retrocaval lymph node metastasis (11 mm short axis) is indicated with white arrows on the CT images, with anatomical location corresponding with the area of increased ²¹²Pb-Compound 1 uptake (dark) on each of the SPECT sections.

FIG. 7 depicts the predicted DMPK properties for Compound 1.

FIG. 8 depicts the predicted DMPK properties for PSMA-617.

### DETAILED DESCRIPTION

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

### I. Definitions

As used in the specification and appended claims, unless specified to the contrary, the following terms have the meaning indicated below.

As used herein and in the appended claims, the singular forms “a,” “an,” and “the” include plural referents unless the context clearly dictates otherwise. Thus, for example, reference to “an agent” includes a plurality of such agents.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

“Amino” refers to the —NH2 radical.

“Nitro” refers to the —NO2 radical.

“Oxo” refers to the ═O radical.

“Imino” refers to the =N—H radical.

“Hydroxy” or “hydroxyl” refers to the —OH radical.

“Alkyl” refers to an optionally substituted straight-chain, or optionally substituted branched-chain saturated hydrocarbon monoradical. An alkyl group can have from one to about twenty carbon atoms, from one to about ten carbon atoms, or from one to six carbon atoms. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₈ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, —NO₂, or —C≡CH. In some embodiments, the alkyl is optionally substituted with oxo, halogen, —CN, —CF₃, —OH, or —OMe. In some embodiments, the alkyl is optionally substituted with halogen. The term “alkylene” refers to a bivalent alkyl.

The term “aryl” refers to a radical comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems. Unless stated otherwise specifically in the specification, an aryl may be optionally substituted, for example, with halogen, amino, alkylamino, aminoalkyl, nitrile, nitro, hydroxyl, alkyl, alkenyl, alkynyl, haloalkyl, heteroalkyl, alkoxy, aryl, cycloalkyl, heterocycloalkyl, heteroaryl, —S(O)₂NH—C₁-C₆alkyl, and the like. In some embodiments, an aryl is optionally substituted with halogen, methyl, ethyl, —CN, —CF₃, —OH, —OMe, —NH₂, —NO₂, —S(O)₂NH₂, —S(O)₂NHCH₃, —S(O)₂NHCH₂CH₃, —S(O)₂NHCH(CH₃)₂, —S(O)₂N(CH₃)₂, or —S(O)₂NHC(CH₃)₃. In some embodiments, an aryl is optionally substituted with halogen, methyl, ethyl, —CN, —CF₃, —OH, or —OMe. In some embodiments, the aryl is optionally substituted with halogen. In some embodiments, the aryl is substituted with alkyl, alkenyl, alkynyl, haloalkyl, or heteroalkyl, wherein each alkyl, alkenyl, alkynyl, haloalkyl, heteroalkyl is independently unsubstituted, or substituted with halogen, methyl, ethyl, —CN, —CF₃, —OH, —OMe, —NH₂, or —NO₂ The term “arylene” refers to a bivalent aryl.

The term “cycloalkyl” refers to a monocyclic or polycyclic non-aromatic radical, wherein each of the atoms forming the ring (i.e., skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₈ cycloalkyl), or three to four carbon atoms (C₃-C₄ cycloalkyl). A cycloalkyl can comprise a fused, spiro or bridged ring system. In some embodiments, the cycloalkyl comprises a fused ring system. In some embodiments, the cycloalkyl comprises a spiro ring system. In some embodiments, the cycloalkyl comprises a bridged ring system. In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen. The term “cycloalkylene” refers to a divalent cycloalkyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂—O—CH₂—, —CH₂—N(alkyl)-CH₂—, —CH₂—N(aryl)-CH₂—, —OCH₂CH₂O—, —OCH₂CH₂OCH₂CH₂O—, or —OCH₂CH₂OCH₂CH₂OCH₂CH₂O—. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen. The term “heteroalkylene” refers to a bivalent heteroalkyl.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF3, —OH, or —OMe. In some embodiments, the heterocycloalkyl is optionally substituted with halogen. The term “heterocycloalkylene” refers to a bivalent heterocycloalkyl.

“Heteroaryl” refers to a ring system radical comprising carbon atom(s) and one or more ring heteroatoms that selected from the group consisting of nitrogen, oxygen, phosphorous, and sulfur, and at least one aromatic ring. In some embodiments, heteroaryl is monocyclic, bicyclic or polycyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₈ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7,8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, spiro or bridged ring systems. Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH2, or —NO₂. In some embodiments, a heteroaryl is optionally substituted with halogen, methyl, ethyl, —CN, —CF3, —OH, or —OMe. In some embodiments, the heteroaryl is optionally substituted with halogen. The term “heteroarylene” refers to a bivalent heteroaryl.

The term “optionally substituted” or “substituted” means that the referenced group is optionally substituted with one or more additional group(s) individually and independently selected from D, halogen, —CN, oxo, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some embodiments, optional substituents are independently selected from D, halogen, —CN, oxo, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. When indicating the number of substituents, the term “one or more” means from one substituent to the highest possible number of substitutions, i.e. replacement of one hydrogen up to replacement of all hydrogens by substituents.

The term “unsubstituted” means that the specified group bears no substituents.

The term “moiety” refers to a specific segment or functional group of a molecule. Chemical moieties are often recognized chemical entities embedded in or appended to a molecule.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%. Furthermore, the treatment, prevention, amelioration, or inhibition provided by the methods disclosed herein can include treatment, prevention, amelioration, or inhibition of one or more conditions or symptoms of the disorder, e.g., cancer or an inflammatory disease. Also, for purposes herein, “treatment,” “prevention,” “amelioration,” or “inhibition” encompass delaying the onset of the disorder, or a symptom or condition thereof. As used herein, “treating” includes the concepts of “alleviating”, which refers to lessening the frequency of occurrence or recurrence, or the severity, of any symptoms or other ill effects related to a disorder and/or the associated side effects. The term “treating” also encompasses the concept of “managing” which refers to reducing the severity of a particular disease or disorder in a patient or delaying its recurrence, e.g., lengthening the period of remission in a patient who had suffered from the disease.

As used herein, the term “prevent,” “preventing,” or “prevention” means prevention of the occurrence or onset of one or more symptoms associated with a particular disorder and does not necessarily mean the complete prevention of a disorder. For example, the term “prevent,” “preventing,” and “prevention” refers to the administration of therapy on a prophylactic or preventative basis to an individual who may ultimately, without treatment, manifest at least one symptom of a disease or condition but who has not yet done so. Such individuals can be identified since there are risk factors that are known to correlate with the subsequent occurrence of the disease. Alternatively, prevention therapy can be administered without prior identification of a risk factor, as a prophylactic measure. Delaying the onset of the at least one symptom can also be considered prevention or prophylaxis.

The term “therapeutically effective amount” as used herein to refer to an amount effective at the dosage and duration necessary to achieve the desired therapeutic result. A therapeutically effective amount of the composition may vary depending on factors such as the individual's condition, age, sex, and weight, the radiopharmaceutical conjugate administered, and the route of administration. A therapeutically effective amount can also be an amount that exceeds any toxic or deleterious effect of the composition that would have a beneficial effect on the treatment.

Certain compounds described herein may exist in tautomeric forms, and all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction. Further, ranges described herein such as a range “between 1 and 50” encompass the end points of the ranges. For example, a range “between 10 μg and 100 mg” encompasses both 10 μg and 100 mg.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a companion animal such as a dog or a cat. In one aspect, the mammal is a human. In one aspect, the subject or patient is a human.

### II. Technical Overview

Targeted Radiopharmaceuticals (TRP) are a new generation of nuclear medicine for cancer treatment, imaging, or diagnosis. A TRP can selectively deliver high concentrations of radionuclide-containing molecules to the target cells such as a tumor, and no or very low concentrations to the undesired cells present in normal, healthy tissues. The process can be achieved by engineering the drug molecule with the high-affinity binder (e.g., targeting ligands) and linking it to the radioactive isotope. The biological targets of these binders are highly expressed on tumor cells and have low or no expression in healthy tissues and organs. When the radioisotope decays, it emits highly energic ionizing radiation in form of alpha, beta, and/or gamma particles. The released energy at the target sites can cause damage or death of the target tissues or be visualized by imaging scanner to achieve therapeutic, imaging, or diagnostic purposes.

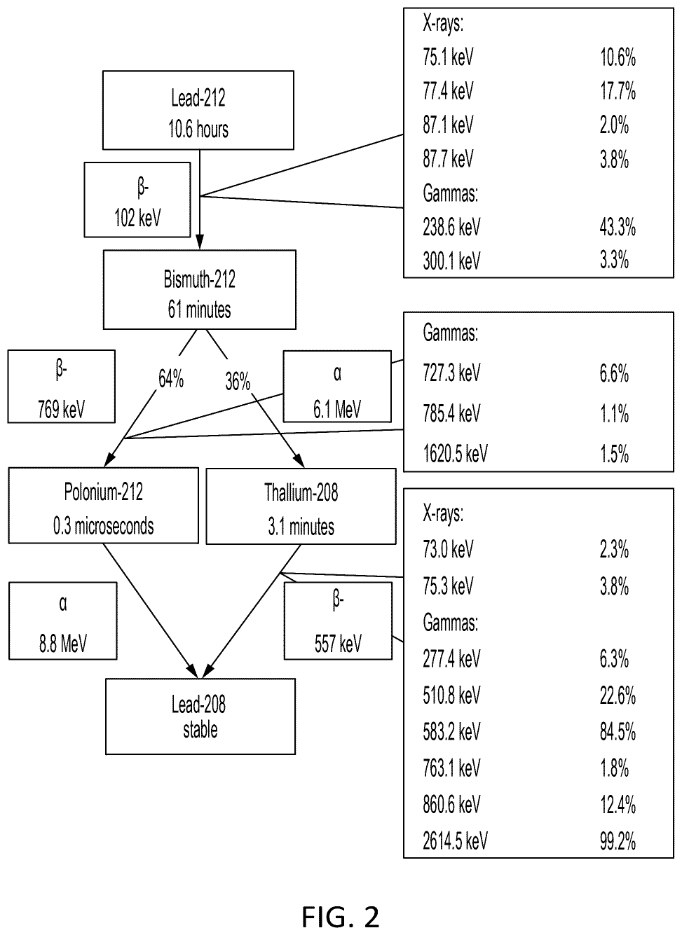

FIG. 2 shows the decay chain of a ²¹²Pb radionuclide. ²¹²Pb has a 10.6 hour half-life and decays to alpha-particle emitting daughter ions ²¹²Bi and ²¹²Po. Eventually, the decayed daughters accumulate in the form of stable ²⁰⁸Pb. The ²¹²Bi and ²¹²Po daughter ions emit a 6.1 MeV and 8.8 MeV alpha-particle, respectively.

### III. Radiopharmaceutical Conjugate

In one aspect, provided herein are radiopharmaceutical conjugates comprising:
(a) a metal chelator;
(b) a target-binding moiety; and
(c) a radionuclide bound to the metal chelator, wherein the radionuclide is ²¹²Pb.
   In some embodiments, the radiopharmaceutical conjugate comprises a structure of Formula (I):

TL-L-R^{M} Formula (I)
or a pharmaceutically acceptable salt thereof, wherein:
TL is a PSMA targeting ligand comprising a urea;
L is a bivalent linking moiety; and
R^{M} is a metal chelator.

In some embodiments of Formula (I), TL is

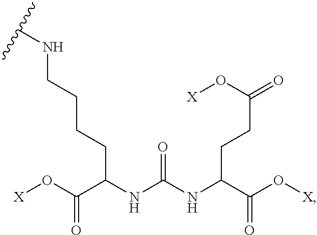

wherein each X is independently selected from the group consisting of H and C₁-C₄alkyl. In some embodiments, TL is

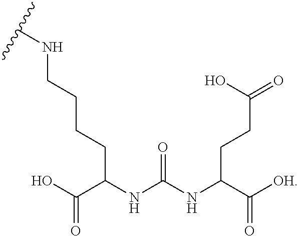 [SMILES: *NCCCCC(NC(=O)NC(CCC(=O)O)C(=O)O)C(=O)O]

In some embodiments TL is

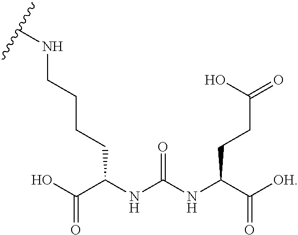 [SMILES: *NCCCC[C@H](NC(=O)N[C@@H](CCC(=O)O)C(=O)O)C(=O)O]

In some embodiments of Formula (I), the a metal chelator R^{M} is a metal chelator described herein. In some embodiments of Formula (I), R^{M} is TCMC.

In some embodiments of Formula (I), R^{M} has the structure of Formula (M-1):

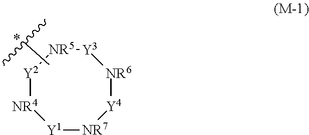

wherein:
Y¹, Y², Y³, and Y⁴ are each independently an optionally substituted —C₁₋₆alkylene-;
R⁴, R⁵, R⁶, and R⁷ are each independently selected from the group consisting of —C₁₋₁₀alkylene-C(═O)OR³, —C₁₋₁₀alkylene-C(═O)N(R³)₂, —C₁₋₁₀alkylene-P(═O)(OR³)₂, —C₁₋₁₀alkylene-P(═O)OR³(R³), and —C₁₋₁₀alkylene-P(═O)(R³)₂; or one of R⁴ and R⁶ or R⁵ and R⁷ together from a —(CH₂)ₘ—bridge; wherein each C₁₋₁₀alkylene is optionally substituted;
each R³ is independently selected from the group consisting of H and C₁₋₆alkyl, wherein the C₁₋₆alkyl is optionally substituted;
m is 1 to 3; and
* is attached to L.

In some embodiments of Formula (I), the radiopharmaceutical conjugate comprises a structure of Formula (I-A):

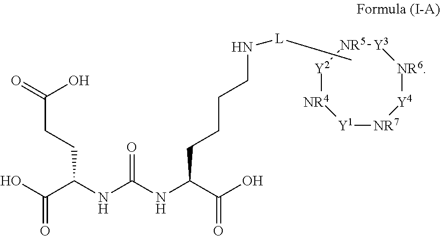

In some embodiments of Formula (I) or Formula (I-A), Y¹, Y², Y³, and Y⁴ are each independently an optionally substituted —C₂alkylene-.

In some embodiments of Formula (I) or Formula (I-A), the radiopharmaceutical conjugate comprises a structure of Formula (I-A-1):

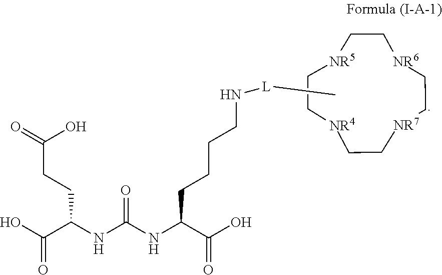

In some embodiments of Formula (I) or Formula (I-A), the radiopharmaceutical conjugate comprises a structure of Formula (I-B-1):

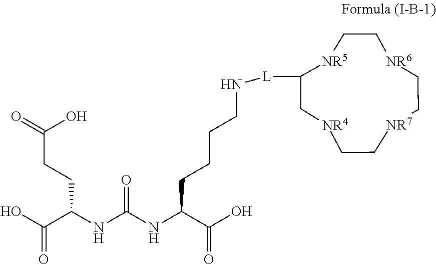

wherein R⁴, R⁵, R⁶, and R⁷ are each independently selected from the group consisting of —C₁₋₃alkylene-C(═O)OR³, and —C₁₋₃alkylene-C(═O)N(R³)₂, wherein each C₁₋₃alkylene is optionally substituted with —C(═O)OH.

In some embodiments of Formula (I-B-1), R⁴, R⁵, R⁶, and R⁷ are each —C₁alkylene-C(═O)NH₂.

In some embodiments of Formula (I) or Formula (I-A), the radiopharmaceutical conjugate comprises a structure of Formula (I-B-2):

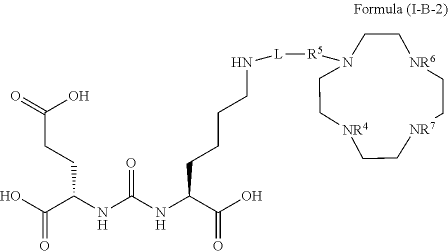

wherein R¹ is —C₁₋₃alkylene-C(═O)—, wherein the C₁₋₃alkylene is optionally substituted with —C(═O)OH; and
R⁴, R⁶, and R⁷ are each independently selected from the group consisting of —C₁₋₃alkylene-C(═O)OR³, and —C₁₋₃alkylene-C(═O)N(R³)₂, wherein each C₁₋₃alkylene is optionally substituted with —C(═O)OH.

In some embodiments of Formula (I-B-2), R⁴, R⁶, and R⁷ are each —C₁alkylene-C(═O)NH₂, such as —CH₂—C(═O)NH₂.

In some embodiments of Formula (I), Formula (I-A), Formula (I-A-1), Formula (I-B-1), Formula (I-B-1), or Formula (I-B-2), L comprises an optionally substituted C₁₋₁₀heteroalkylene. In some embodiments, the heteroalkylene comprises at least one nitrogen atom; and:
wherein the heteroalkylene is optionally substituted with one or more substituents selected from

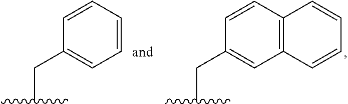 [SMILES: *Cc1ccccc1 ; *Cc1ccc2ccccc2c1]

each of which is optionally substituted with C₁₋₃alkyl, halogen, and —OH.
   In some embodiments, the heteroalkylene is substituted with

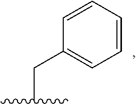 [SMILES: *Cc1ccccc1]

which is optionally substituted with C₁₋₃alkyl, halogen, and —OH. In some embodiments, the heteroalkylene is substituted with

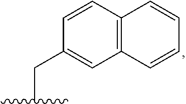 [SMILES: *Cc1ccc2ccccc2c1]

which is optionally substituted with C₁₋₃alkyl, halogen, and —OH.

In some embodiments of Formula (I), Formula (I-A), Formula (I-A-1), Formula (I-B-1), Formula (I-B-1), or Formula (I-B-2), L has the structure of:

-L1-X1-L2-X2-L3-X3-L4-X4-L5-
wherein,
L1, L2, L3, L4, and L5 are each independently selected from a bond, —O—, —S—, —C(═O)—, —C(═O)NR³—, —NR³—, —C(═O)O—, —C(═O)S—, —S(═O)₂—, —S(═O)₂NR³—, S(═O)NR³—, —OS(═O)₂—, —N(R³)C(═S)N(R³)—, and —N(R³)C(═O)N(R³)—;
X1, X2, X3, and X4 are each independently selected from a bond, a structure of

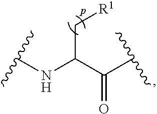

C₁₋₁₀alkylene-, —C₁₋₁₀heteroalkylene-, —C₅₋₇cycloalkylene-, —C₁₋₁₀alkylene-C₅₋₇cycloalkylene-, —C₆arylene-, —C₁₋₁₀alkylene-C₆arylene-, −5 to 7-membered heterocycloalkylene-, —C₁₋₁₀alkylene-5 to 7-membered heterocycloalkylene-, 5 to 7-membered heteroarylene-, and —C₁₋₁₀alkylene-5 to 7-membered heteroarylene-, wherein each alkylene and heteroalkylene is independently optionally substituted;
p is 0, 1, 2, 3, or 4; and
R¹ is —H, —OH, —OMe, —SH, —SMe, —NH₂, —C(═O)OH, —C(═O)NH₂, C₆₋₁₀ aryl, or 5 to 10-membered heteroaryl, wherein each aryl and heteroaryl are independently optionally substituted with one or more substituents selected from halogen and —OH;

In some embodiments, the alkylene and heteroalkylene of X1, X2, X3, and/or X4 is independently, optionally substituted with —C(═O)OH.

In some embodiments, the radiopharmaceutical conjugate comprises a compound having the structure listed in Table 1, or a pharmaceutically acceptable salt thereof.

TABLE 1

| Cpd | Structure |
|---|---|
| 1 | 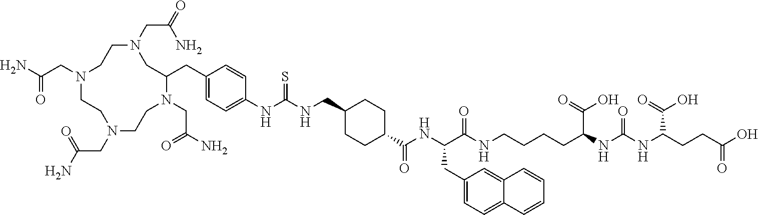 |
| 2 | 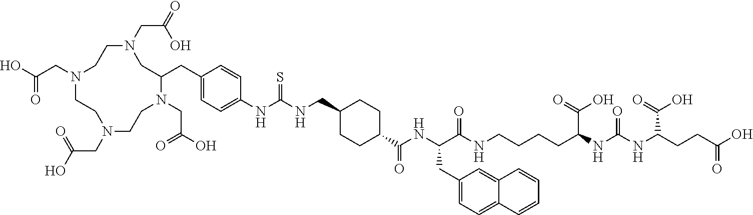 |
| 3 | 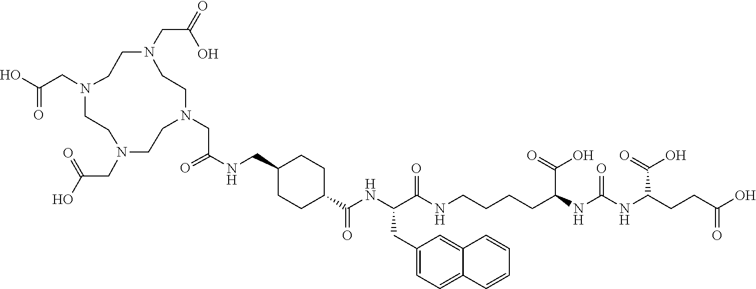 |
| 4 | 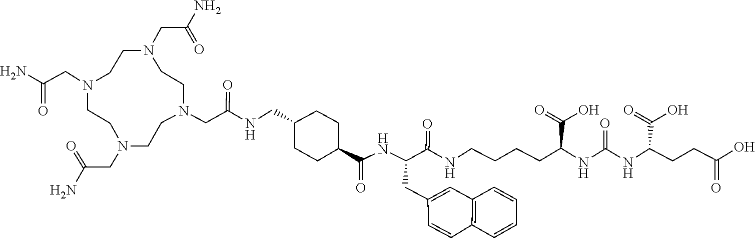 [SMILES: NC(=O)CN1CCN(CC(N)=O)CCN(CC(=O)NC[C@H]2CC[C@H](C(=O)N[C@@H](Cc3ccc4ccccc4c3)C(=O)NCCCC[C@H](NC(=O)N[C@@H](CCC(=O)O)C(=O)O)C(=O)O)CC2)CCN(CC(N)=O)CC1] |
| 5 | 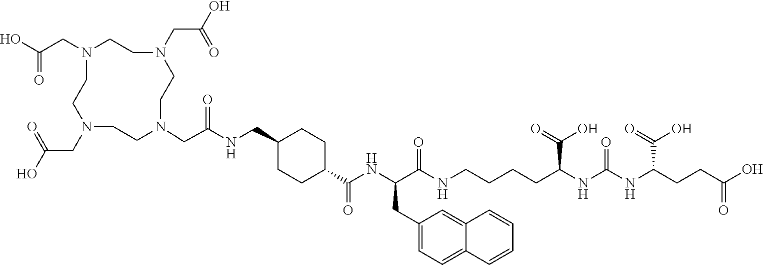 [SMILES: O=C(O)CC[C@H](NC(=O)N[C@@H](CCCCNC(=O)[C@@H](Cc1ccc2ccccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O] |
| 6 | 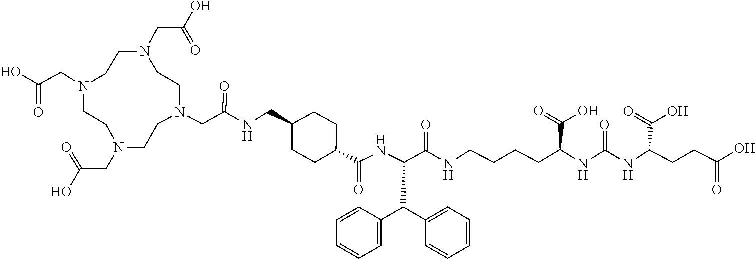 [SMILES: O=C(O)CC[C@H](NC(=O)N[C@@H](CCCCNC(=O)[C@@H](NC(=O)[C@H]1CC[C@H](CNC(=O)CN2CCN(CC(=O)O)CCN(CC(=O)O)CCN(CC(=O)O)CC2)CC1)C(c1ccccc1)c1ccccc1)C(=O)O)C(=O)O] |
| 7 | 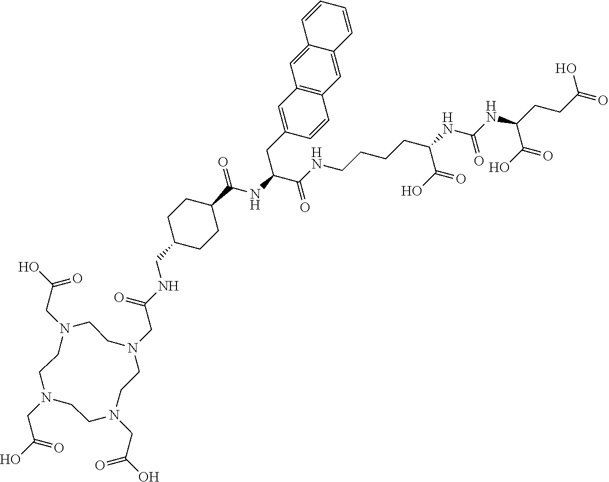 [SMILES: O=C(O)CC[C@H](NC(=O)N[C@@H](CCCCNC(=O)[C@H](Cc1ccc2cc3ccccc3cc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O] |
| 8 | 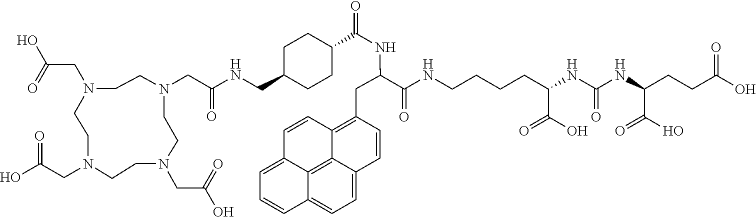 [SMILES: O=C(O)CC[C@H](NC(=O)N[C@@H](CCCCNC(=O)C(Cc1ccc2ccc3cccc4ccc1c2c34)NC(=O)[C@H]1CC[C@H](CNC(=O)CN2CCN(CC(=O)O)CCN(CC(=O)O)CCN(CC(=O)O)CC2)CC1)C(=O)O)C(=O)O] |
| 9 | 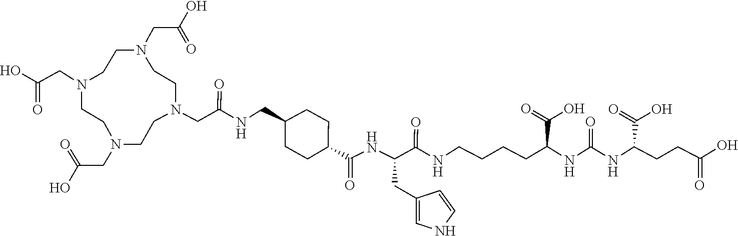 [SMILES: O=C(O)CC[C@H](NC(=O)N[C@@H](CCCCNC(=O)[C@H](Cc1cc[nH]c1)NC(=O)[C@H]1CC[C@H](CNC(=O)CN2CCN(CC(=O)O)CCN(CC(=O)O)CCN(CC(=O)O)CC2)CC1)C(=O)O)C(=O)O] |
| 10 | 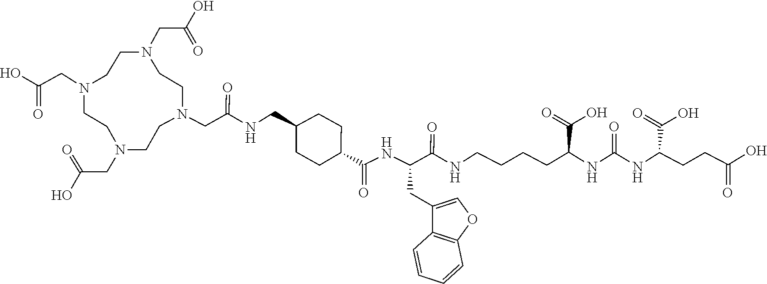 [SMILES: O=C(O)CC[C@H](NC(=O)N[C@@H](CCCCNC(=O)[C@H](Cc1coc2ccccc12)NC(=O)[C@H]1CC[C@H](CNC(=O)CN2CCN(CC(=O)O)CCN(CC(=O)O)CCN(CC(=O)O)CC2)CC1)C(=O)O)C(=O)O] |
| 11 | 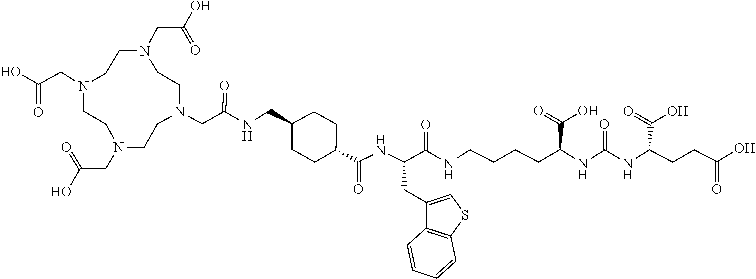 [SMILES: O=C(O)CC[C@H](NC(=O)N[C@@H](CCCCNC(=O)[C@H](Cc1csc2ccccc12)NC(=O)[C@H]1CC[C@H](CNC(=O)CN2CCN(CC(=O)O)CCN(CC(=O)O)CCN(CC(=O)O)CC2)CC1)C(=O)O)C(=O)O] |
| 11 | 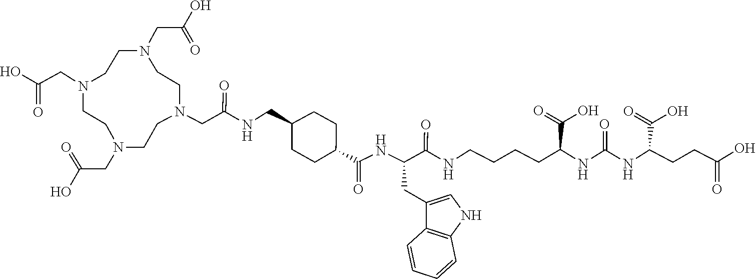 [SMILES: O=C(O)CC[C@H](NC(=O)N[C@@H](CCCCNC(=O)[C@H](Cc1c[nH]c2ccccc12)NC(=O)[C@H]1CC[C@H](CNC(=O)CN2CCN(CC(=O)O)CCN(CC(=O)O)CCN(CC(=O)O)CC2)CC1)C(=O)O)C(=O)O] |
| 12 | 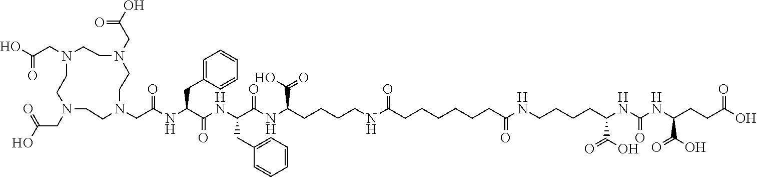 [SMILES: O=C(O)CC[C@H](NC(=O)N[C@@H](CCCCNC(=O)CCCCCCC(=O)NCCCC[C@@H](NC(=O)[C@H](Cc1ccccc1)NC(=O)[C@H](Cc1ccccc1)NC(=O)CN1CCN(CC(=O)O)CCN(CC(=O)O)CCN(CC(=O)O)CC1)C(=O)O)C(=O)O)C(=O)O] |
| 13 | 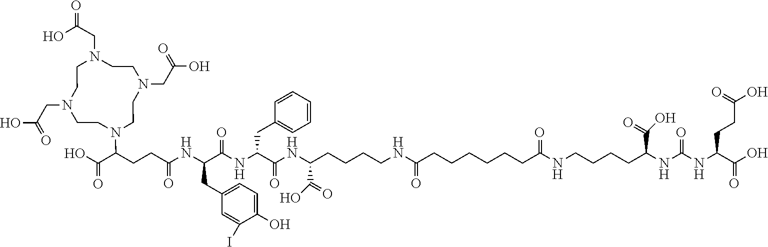 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O] |
| 14 | 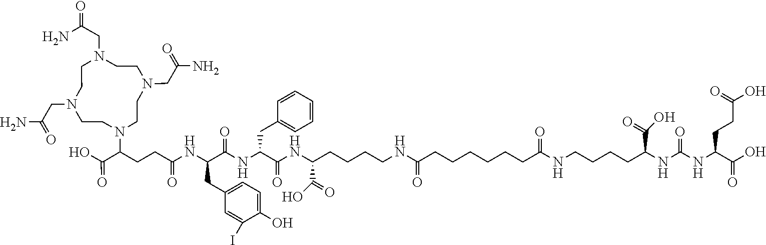 [SMILES: NC(=O)CN1CCN(CC(N)=O)CCN(C(CCC(=O)N[C@H](Cc2ccc(O)c(I)c2)C(=O)N[C@H](Cc2ccccc2)C(=O)N[C@H](CCCCNC(=O)CCCCCCC(=O)NCCCC[C@H](NC(=O)N[C@@H](CCC(=O)O)C(=O)O)C(=O)O)C(=O)O)C(=O)O)CCN(CC(N)=O)CC1] |
| 15 | 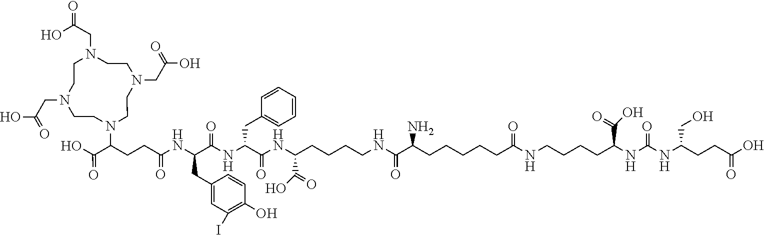 [SMILES: N[C@@H](CCCCCC(=O)NCCCC[C@H](NC(=O)N[C@H](CO)CCC(=O)O)C(=O)O)C(=O)NCCCC[C@@H](NC(=O)[C@@H](Cc1ccccc1)NC(=O)[C@@H](Cc1ccc(O)c(I)c1)NC(=O)CCC(C(=O)O)N1CCN(CC(=O)O)CCN(CC(=O)O)CCN(CC(=O)O)CC1)C(=O)O] |
| 16 | 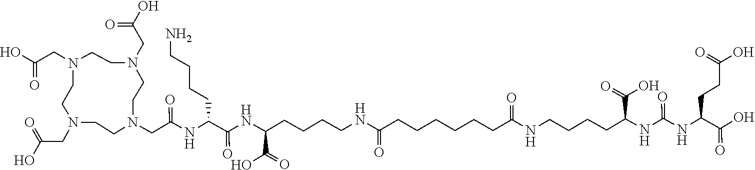 [SMILES: NCCCC[C@@H](NC(=O)CN1CCN(CC(=O)O)CCN(CC(=O)O)CCN(CC(=O)O)CC1)C(=O)N[C@@H](CCCCNC(=O)CCCCCCC(=O)NCCCC[C@H](NC(=O)N[C@@H](CCC(=O)O)C(=O)O)C(=O)O)C(=O)O] |
| 17 | 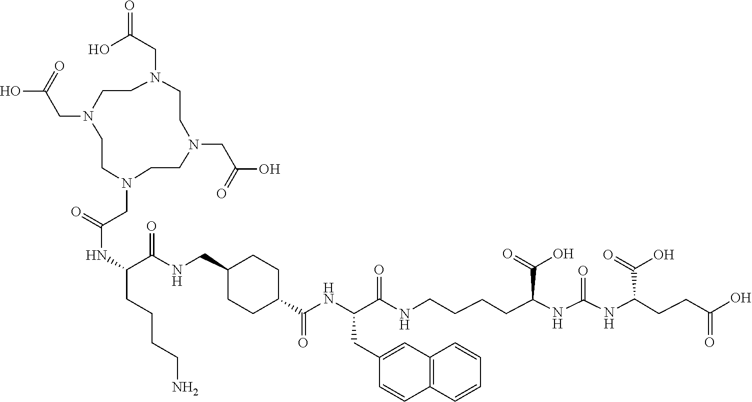 [SMILES: NCCCC[C@H](NC(=O)CN1CCN(CC(=O)O)CCN(CC(=O)O)CCN(CC(=O)O)CC1)C(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 18 | 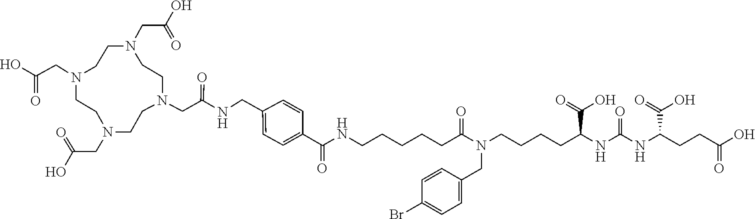 [SMILES: O=C(O)CC[C@H](NC(=O)N[C@@H](CCCCN(Cc1ccc(Br)cc1)C(=O)CCCCCNC(=O)c1ccc(CNC(=O)CN2CCN(CC(=O)O)CCN(CC(=O)O)CCN(CC(=O)O)CC2)cc1)C(=O)O)C(=O)O] |
| 19 | 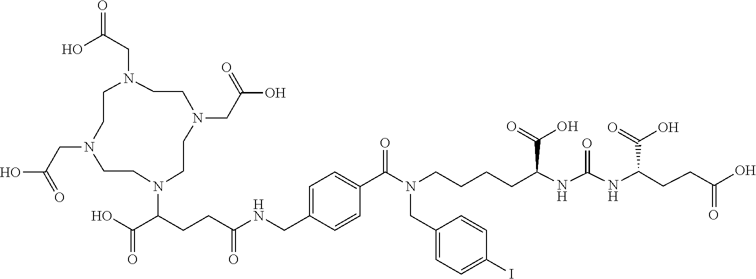 [SMILES: O=C(O)CC[C@H](NC(=O)N[C@@H](CCCCN(Cc1ccc(I)cc1)C(=O)c1ccc(CNC(=O)CCC(C(=O)O)N2CCN(CC(=O)O)CCN(CC(=O)O)CCN(CC(=O)O)CC2)cc1)C(=O)O)C(=O)O] |
| 20 | 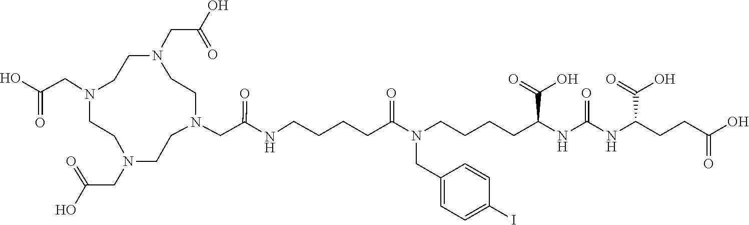 [SMILES: O=C(O)CC[C@H](NC(=O)N[C@@H](CCCCN(Cc1ccc(I)cc1)C(=O)CCCCNC(=O)CN1CCN(CC(=O)O)CCN(CC(=O)O)CCN(CC(=O)O)CC1)C(=O)O)C(=O)O] |
| 21 | 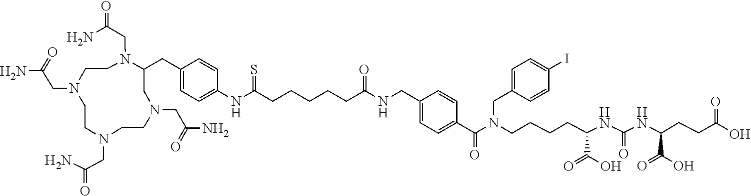 [SMILES: NC(=O)CN1CCN(CC(N)=O)CCN(CC(N)=O)C(Cc2ccc(NC(=S)CCCCCC(=O)NCc3ccc(C(=O)N(CCCC[C@H](NC(=O)N[C@@H](CCC(=O)O)C(=O)O)C(=O)O)Cc4ccc(I)cc4)cc3)cc2)CN(CC(N)=O)CC1] |
| 22 | 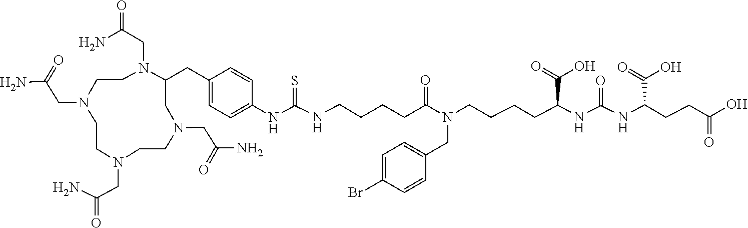 [SMILES: NC(=O)CN1CCN(CC(N)=O)CCN(CC(N)=O)C(Cc2ccc(NC(=S)NCCCCC(=O)N(CCCC[C@H](NC(=O)N[C@@H](CCC(=O)O)C(=O)O)C(=O)O)Cc3ccc(Br)cc3)cc2)CN(CC(N)=O)CC1] |
| 23 | 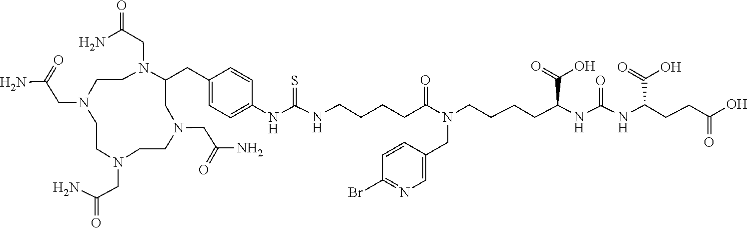 [SMILES: NC(=O)CN1CCN(CC(N)=O)CCN(CC(N)=O)C(Cc2ccc(NC(=S)NCCCCC(=O)N(CCCC[C@H](NC(=O)N[C@@H](CCC(=O)O)C(=O)O)C(=O)O)Cc3ccc(Br)nc3)cc2)CN(CC(N)=O)CC1] |
| 24 | 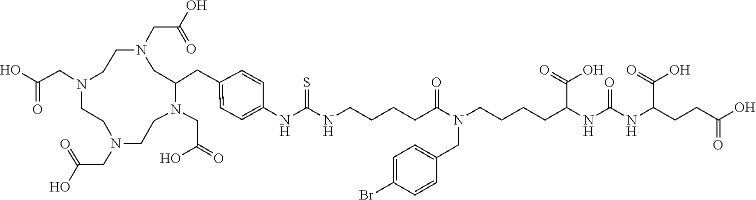 [SMILES: O=C(O)CCC(NC(=O)NC(CCCCN(Cc1ccc(Br)cc1)C(=O)CCCCNC(=S)Nc1ccc(CC2CN(CC(=O)O)CCN(CC(=O)O)CCN(CC(=O)O)CCN2CC(=O)O)cc1)C(=O)O)C(=O)O] |
| 25 | 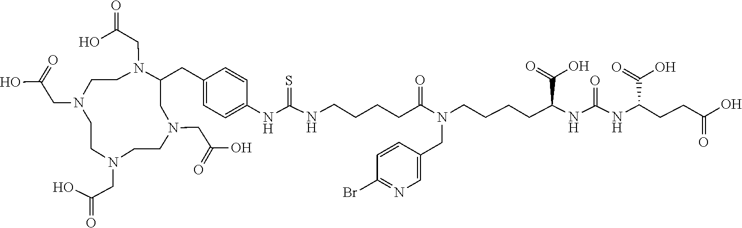 [SMILES: O=C(O)CC[C@H](NC(=O)N[C@@H](CCCCN(Cc1ccc(Br)nc1)C(=O)CCCCNC(=S)Nc1ccc(CC2CN(CC(=O)O)CCN(CC(=O)O)CCN(CC(=O)O)CCN2CC(=O)O)cc1)C(=O)O)C(=O)O] |
| 26 | 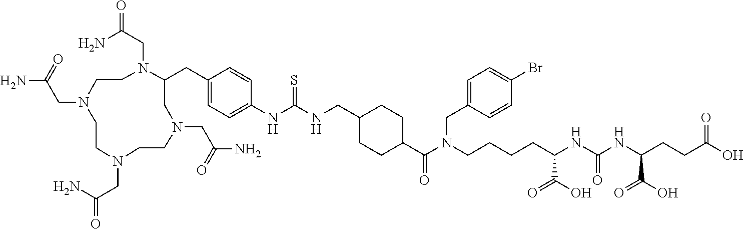 [SMILES: NC(=O)CN1CCN(CC(N)=O)CCN(CC(N)=O)C(Cc2ccc(NC(=S)NCC3CCC(C(=O)N(CCCC[C@H](NC(=O)N[C@@H](CCC(=O)O)C(=O)O)C(=O)O)Cc4ccc(Br)cc4)CC3)cc2)CN(CC(N)=O)CC1] |
| 27 | 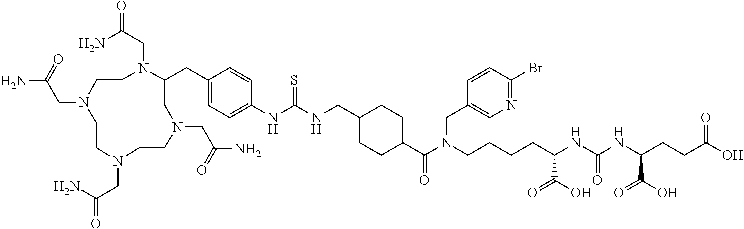 [SMILES: NC(=O)CN1CCN(CC(N)=O)CCN(CC(N)=O)C(Cc2ccc(NC(=S)NCC3CCC(C(=O)N(CCCC[C@H](NC(=O)N[C@@H](CCC(=O)O)C(=O)O)C(=O)O)Cc4ccc(Br)nc4)CC3)cc2)CN(CC(N)=O)CC1] |
| 28 | 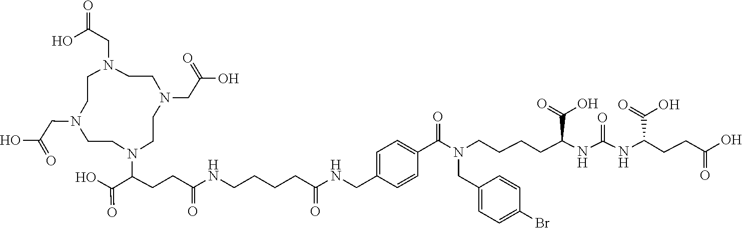 [SMILES: O=C(O)CC[C@H](NC(=O)N[C@@H](CCCCN(Cc1ccc(Br)cc1)C(=O)c1ccc(CNC(=O)CCCCNC(=O)CCC(C(=O)O)N2CCN(CC(=O)O)CCN(CC(=O)O)CCN(CC(=O)O)CC2)cc1)C(=O)O)C(=O)O] |
| 29 | 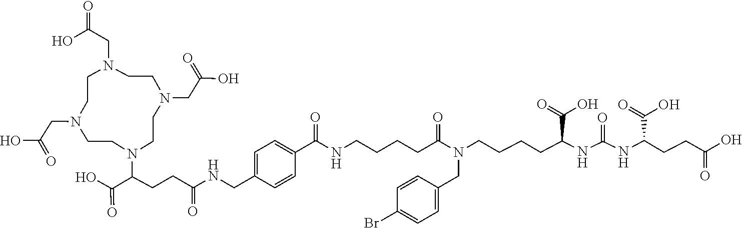 [SMILES: O=C(O)CC[C@H](NC(=O)N[C@@H](CCCCN(Cc1ccc(Br)cc1)C(=O)CCCCNC(=O)c1ccc(CNC(=O)CCC(C(=O)O)N2CCN(CC(=O)O)CCN(CC(=O)O)CCN(CC(=O)O)CC2)cc1)C(=O)O)C(=O)O] |
| 30 | 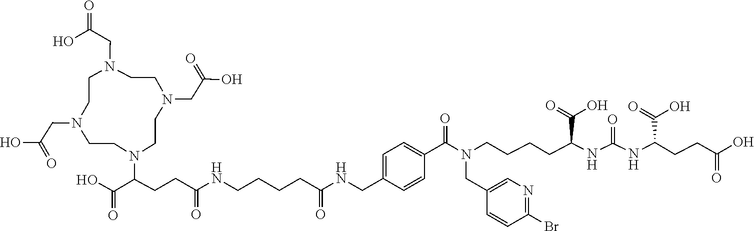 [SMILES: O=C(O)CC[C@H](NC(=O)N[C@@H](CCCCN(Cc1ccc(Br)nc1)C(=O)c1ccc(CNC(=O)CCCCNC(=O)CCC(C(=O)O)N2CCN(CC(=O)O)CCN(CC(=O)O)CCN(CC(=O)O)CC2)cc1)C(=O)O)C(=O)O] |
| 31 | 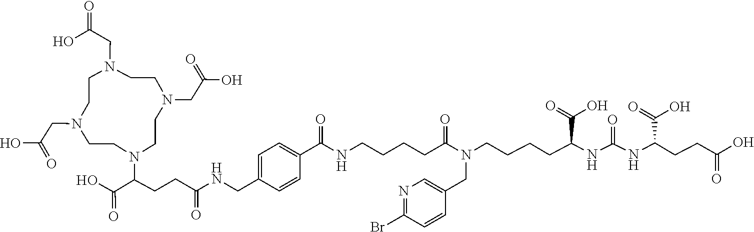 [SMILES: O=C(O)CC[C@H](NC(=O)N[C@@H](CCCCN(Cc1ccc(Br)nc1)C(=O)CCCCNC(=O)c1ccc(CNC(=O)CCC(C(=O)O)N2CCN(CC(=O)O)CCN(CC(=O)O)CCN(CC(=O)O)CC2)cc1)C(=O)O)C(=O)O] |
| 32 | 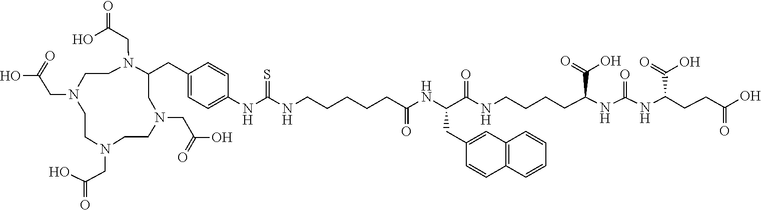 [SMILES: O=C(O)CC[C@H](NC(=O)N[C@@H](CCCCNC(=O)[C@H](Cc1ccc2ccccc2c1)NC(=O)CCCCCNC(=S)Nc1ccc(CC2CN(CC(=O)O)CCN(CC(=O)O)CCN(CC(=O)O)CCN2CC(=O)O)cc1)C(=O)O)C(=O)O] |
| 33 | 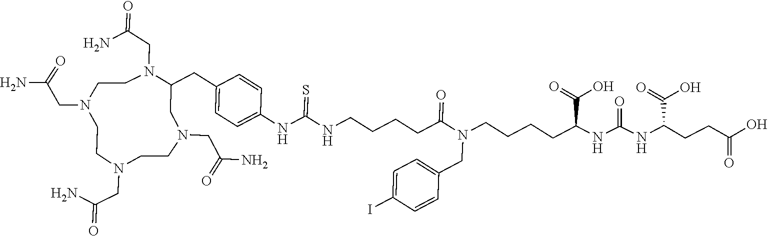 [SMILES: NC(=O)CN1CCN(CC(N)=O)CCN(CC(N)=O)C(Cc2ccc(NC(=S)NCCCCC(=O)N(CCCC[C@H](NC(=O)N[C@@H](CCC(=O)O)C(=O)O)C(=O)O)Cc3ccc(I)cc3)cc2)CN(CC(N)=O)CC1] |
| 34 | 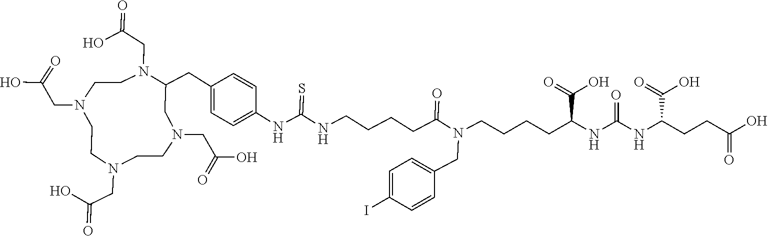 [SMILES: O=C(O)CC[C@H](NC(=O)N[C@@H](CCCCN(Cc1ccc(I)cc1)C(=O)CCCCNC(=S)Nc1ccc(CC2CN(CC(=O)O)CCN(CC(=O)O)CCN(CC(=O)O)CCN2CC(=O)O)cc1)C(=O)O)C(=O)O] |
| 35 | 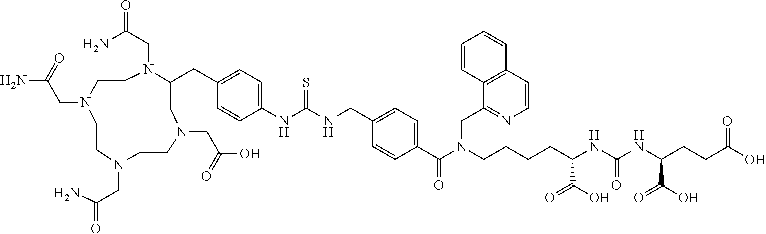 [SMILES: NC(=O)CN1CCN(CC(N)=O)CCN(CC(N)=O)C(Cc2ccc(NC(=S)NCc3ccc(C(=O)N(CCCC[C@H](NC(=O)N[C@@H](CCC(=O)O)C(=O)O)C(=O)O)Cc4nccc5ccccc45)cc3)cc2)CN(CC(=O)O)CC1] |
| 36 | 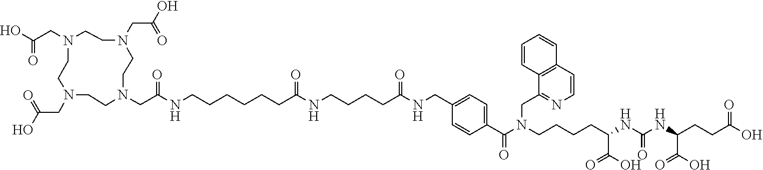 [SMILES: O=C(O)CC[C@H](NC(=O)N[C@@H](CCCCN(Cc1nccc2ccccc12)C(=O)c1ccc(CNC(=O)CCCCNC(=O)CCCCCCNC(=O)CN2CCN(CC(=O)O)CCN(CC(=O)O)CCN(CC(=O)O)CC2)cc1)C(=O)O)C(=O)O] |
| 37 | 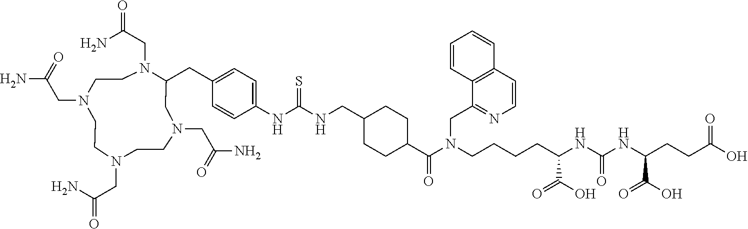 [SMILES: NC(=O)CN1CCN(CC(N)=O)CCN(CC(N)=O)C(Cc2ccc(NC(=S)NCC3CCC(C(=O)N(CCCC[C@H](NC(=O)N[C@@H](CCC(=O)O)C(=O)O)C(=O)O)Cc4nccc5ccccc45)CC3)cc2)CN(CC(N)=O)CC1] |
| 38 | 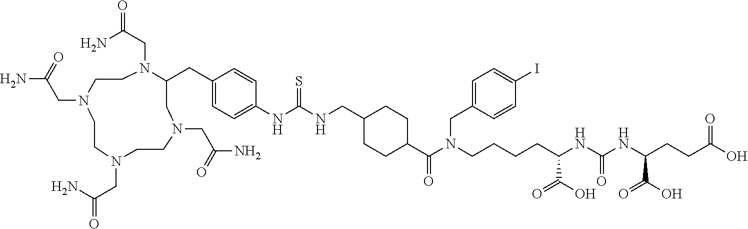 [SMILES: NC(=O)CN1CCN(CC(N)=O)CCN(CC(N)=O)C(Cc2ccc(NC(=S)NCC3CCC(C(=O)N(CCCC[C@H](NC(=O)N[C@@H](CCC(=O)O)C(=O)O)C(=O)O)Cc4ccc(I)cc4)CC3)cc2)CN(CC(N)=O)CC1] |
| 39 | 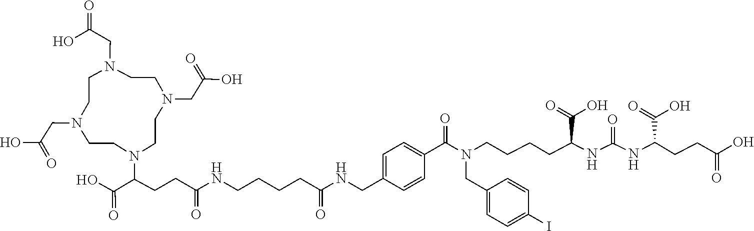 [SMILES: O=C(O)CC[C@H](NC(=O)N[C@@H](CCCCN(Cc1ccc(I)cc1)C(=O)c1ccc(CNC(=O)CCCCNC(=O)CCC(C(=O)O)N2CCN(CC(=O)O)CCN(CC(=O)O)CCN(CC(=O)O)CC2)cc1)C(=O)O)C(=O)O] |
| 40 | 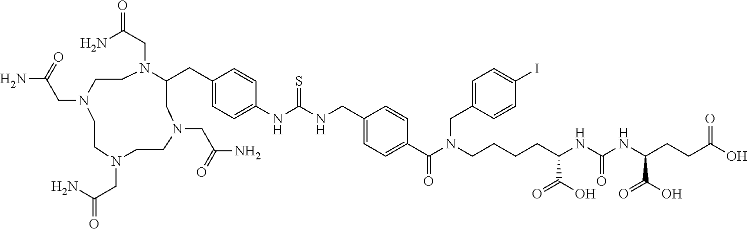 [SMILES: NC(=O)CN1CCN(CC(N)=O)CCN(CC(N)=O)C(Cc2ccc(NC(=S)NCc3ccc(C(=O)N(CCCC[C@H](NC(=O)N[C@@H](CCC(=O)O)C(=O)O)C(=O)O)Cc4ccc(I)cc4)cc3)cc2)CN(CC(N)=O)CC1] |
| 41 | 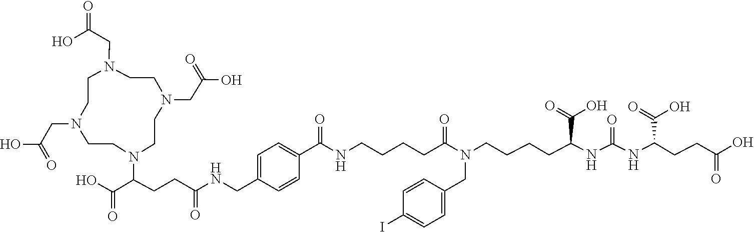 [SMILES: O=C(O)CC[C@H](NC(=O)N[C@@H](CCCCN(Cc1ccc(I)cc1)C(=O)CCCCNC(=O)c1ccc(CNC(=O)CCC(C(=O)O)N2CCN(CC(=O)O)CCN(CC(=O)O)CCN(CC(=O)O)CC2)cc1)C(=O)O)C(=O)O] |
| 42 | 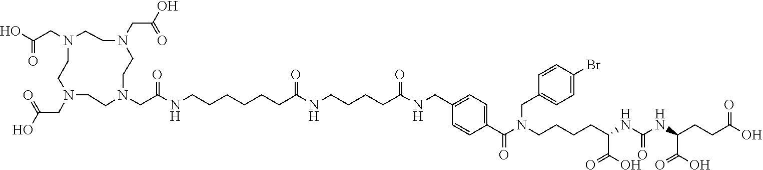 [SMILES: O=C(O)CC[C@H](NC(=O)N[C@@H](CCCCN(Cc1ccc(Br)cc1)C(=O)c1ccc(CNC(=O)CCCCNC(=O)CCCCCCNC(=O)CN2CCN(CC(=O)O)CCN(CC(=O)O)CCN(CC(=O)O)CC2)cc1)C(=O)O)C(=O)O] |
| 43 | 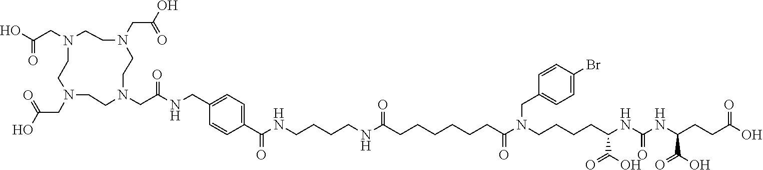 [SMILES: O=C(O)CC[C@H](NC(=O)N[C@@H](CCCCN(Cc1ccc(Br)cc1)C(=O)CCCCCCC(=O)NCCCCNC(=O)c1ccc(CNC(=O)CN2CCN(CC(=O)O)CCN(CC(=O)O)CCN(CC(=O)O)CC2)cc1)C(=O)O)C(=O)O] |
| 44 | 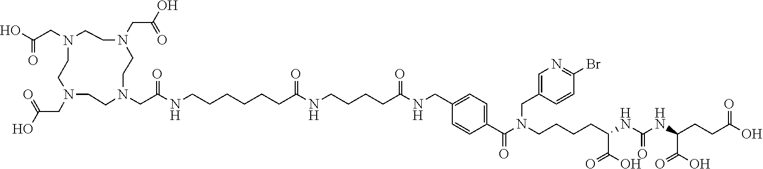 [SMILES: O=C(O)CC[C@H](NC(=O)N[C@@H](CCCCN(Cc1ccc(Br)nc1)C(=O)c1ccc(CNC(=O)CCCCNC(=O)CCCCCCNC(=O)CN2CCN(CC(=O)O)CCN(CC(=O)O)CCN(CC(=O)O)CC2)cc1)C(=O)O)C(=O)O] |
| 45 | 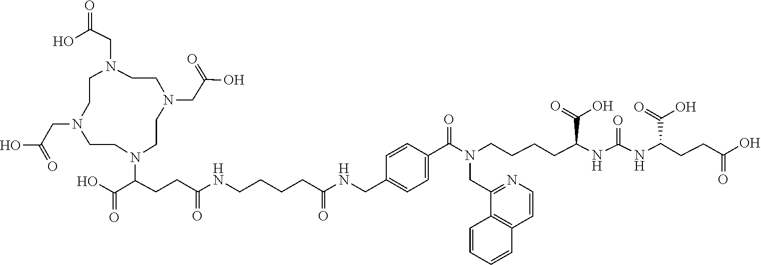 [SMILES: O=C(O)CC[C@H](NC(=O)N[C@@H](CCCCN(Cc1nccc2ccccc12)C(=O)c1ccc(CNC(=O)CCCCNC(=O)CCC(C(=O)O)N2CCN(CC(=O)O)CCN(CC(=O)O)CCN(CC(=O)O)CC2)cc1)C(=O)O)C(=O)O] |
| 46 | 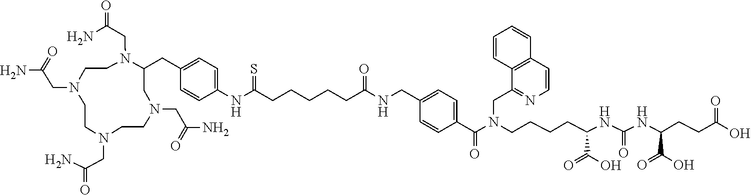 [SMILES: NC(=O)CN1CCN(CC(N)=O)CCN(CC(N)=O)C(Cc2ccc(NC(=S)CCCCCC(=O)NCc3ccc(C(=O)N(CCCC[C@H](NC(=O)N[C@@H](CCC(=O)O)C(=O)O)C(=O)O)Cc4nccc5ccccc45)cc3)cc2)CN(CC(N)=O)CC1] |
| 47 | 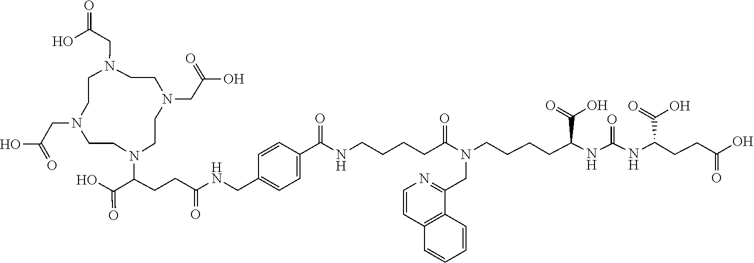 [SMILES: O=C(O)CC[C@H](NC(=O)N[C@@H](CCCCN(Cc1nccc2ccccc12)C(=O)CCCCNC(=O)c1ccc(CNC(=O)CCC(C(=O)O)N2CCN(CC(=O)O)CCN(CC(=O)O)CCN(CC(=O)O)CC2)cc1)C(=O)O)C(=O)O] |
| 48 | 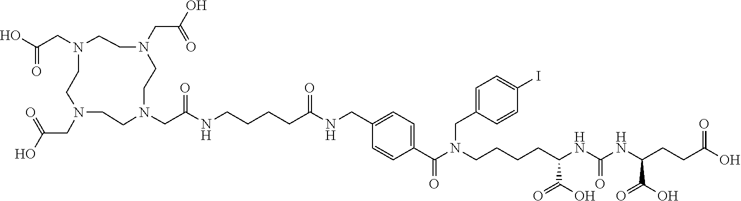 [SMILES: O=C(O)CC[C@H](NC(=O)N[C@@H](CCCCN(Cc1ccc(I)cc1)C(=O)c1ccc(CNC(=O)CCCCNC(=O)CN2CCN(CC(=O)O)CCN(CC(=O)O)CCN(CC(=O)O)CC2)cc1)C(=O)O)C(=O)O] |
| 49 | 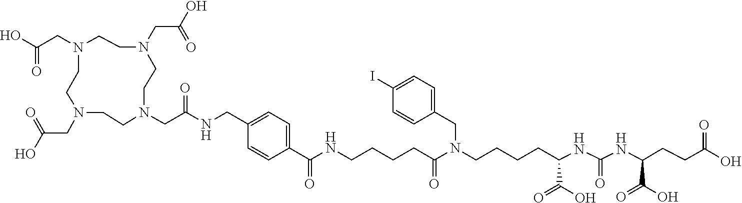 [SMILES: O=C(O)CC[C@H](NC(=O)N[C@@H](CCCCN(Cc1ccc(I)cc1)C(=O)CCCCNC(=O)c1ccc(CNC(=O)CN2CCN(CC(=O)O)CCN(CC(=O)O)CCN(CC(=O)O)CC2)cc1)C(=O)O)C(=O)O] |
| 50 | 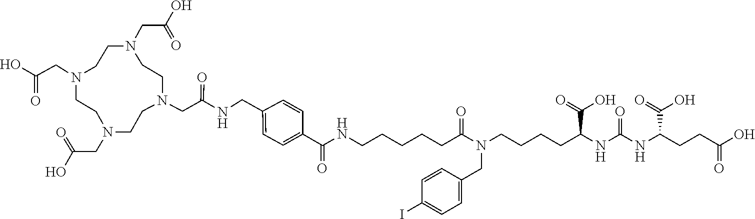 [SMILES: O=C(O)CC[C@H](NC(=O)N[C@@H](CCCCN(Cc1ccc(I)cc1)C(=O)CCCCCNC(=O)c1ccc(CNC(=O)CN2CCN(CC(=O)O)CCN(CC(=O)O)CCN(CC(=O)O)CC2)cc1)C(=O)O)C(=O)O] |
| 51 | 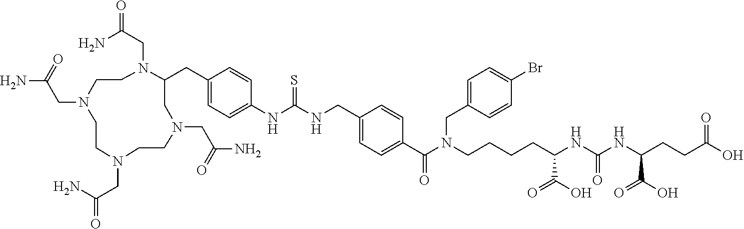 [SMILES: NC(=O)CN1CCN(CC(N)=O)CCN(CC(N)=O)C(Cc2ccc(NC(=S)NCc3ccc(C(=O)N(CCCC[C@H](NC(=O)N[C@@H](CCC(=O)O)C(=O)O)C(=O)O)Cc4ccc(Br)cc4)cc3)cc2)CN(CC(N)=O)CC1] |
| 52 | 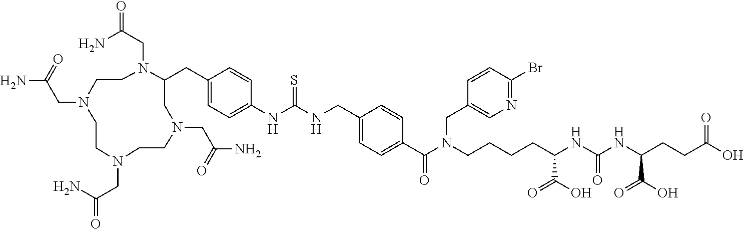 [SMILES: NC(=O)CN1CCN(CC(N)=O)CCN(CC(N)=O)C(Cc2ccc(NC(=S)NCc3ccc(C(=O)N(CCCC[C@H](NC(=O)N[C@@H](CCC(=O)O)C(=O)O)C(=O)O)Cc4ccc(Br)nc4)cc3)cc2)CN(CC(N)=O)CC1] |
| 53 | 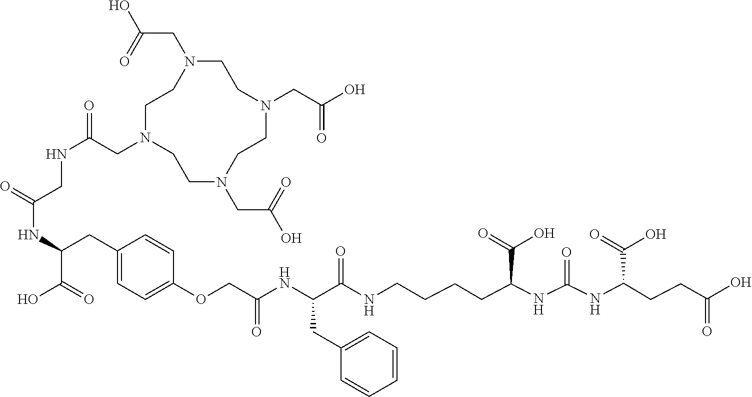 [SMILES: O=C(O)CC[C@H](NC(=O)N[C@@H](CCCCNC(=O)[C@H](Cc1ccccc1)NC(=O)COc1ccc(C[C@H](NC(=O)CNC(=O)CN2CCN(CC(=O)O)CCN(CC(=O)O)CCN(CC(=O)O)CC2)C(=O)O)cc1)C(=O)O)C(=O)O] |
| 54 | 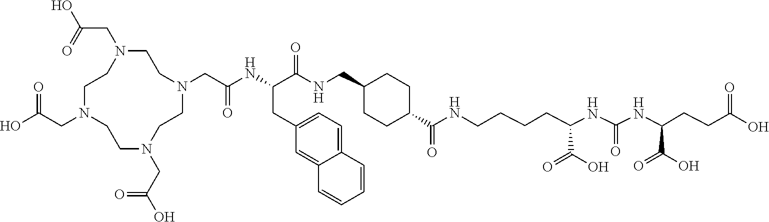 [SMILES: O=C(O)CC[C@H](NC(=O)N[C@@H](CCCCNC(=O)[C@H]1CC[C@H](CNC(=O)[C@H](Cc2ccc3ccccc3c2)NC(=O)CN2CCN(CC(=O)O)CCN(CC(=O)O)CCN(CC(=O)O)CC2)CC1)C(=O)O)C(=O)O] |
| 55 | 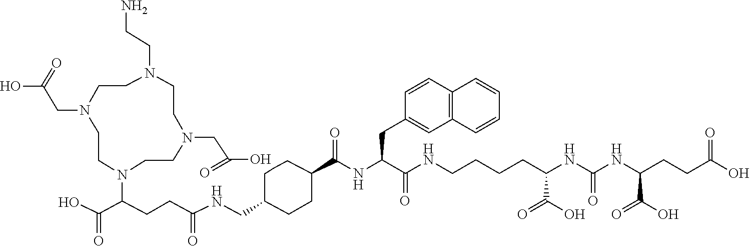 [SMILES: NCCN1CCN(CC(=O)O)CCN(C(CCC(=O)NC[C@@H]2CCC[C@@H](C(=O)N[C@@H](Cc3ccc4ccccc4c3)C(=O)NCCCC[C@H](NC(=O)N[C@@H](CCC(=O)O)C(=O)O)C(=O)O)CC2)C(=O)O)CCN(CC(=O)O)CC1] |
| 56 | 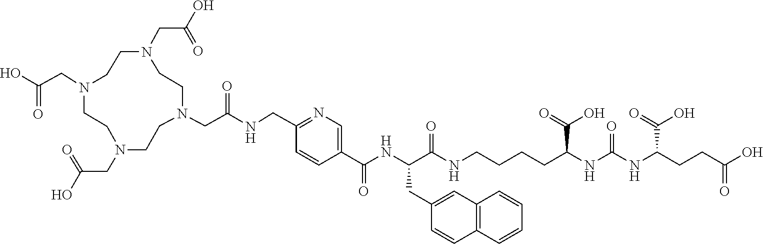 [SMILES: O=C(O)CC[C@H](NC(=O)N[C@@H](CCCCNC(=O)[C@H](Cc1ccc2ccccc2c1)NC(=O)c1ccc(CNC(=O)CN2CCN(CC(=O)O)CCN(CC(=O)O)CCN(CC(=O)O)CC2)nc1)C(=O)O)C(=O)O] |
| 57 | 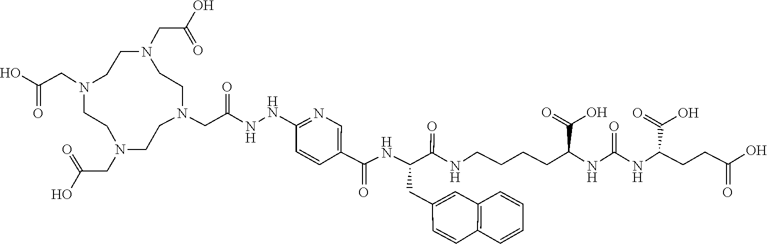 [SMILES: O=C(O)CC[C@H](NC(=O)N[C@@H](CCCCNC(=O)[C@H](Cc1ccc2ccccc2c1)NC(=O)c1ccc(NNC(=O)CN2CCN(CC(=O)O)CCN(CC(=O)O)CCN(CC(=O)O)CC2)nc1)C(=O)O)C(=O)O] |
| 58 | 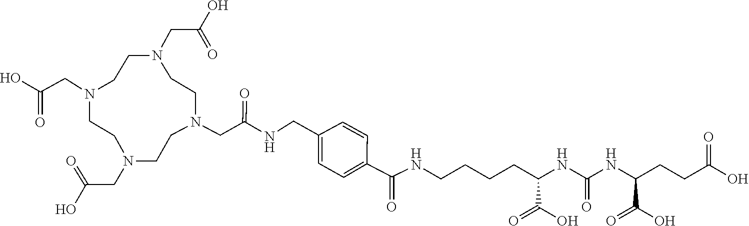 [SMILES: O=C(O)CC[C@H](NC(=O)N[C@@H](CCCCNC(=O)c1ccc(CNC(=O)CN2CCN(CC(=O)O)CCN(CC(=O)O)CCN(CC(=O)O)CC2)cc1)C(=O)O)C(=O)O] |
| 59 | 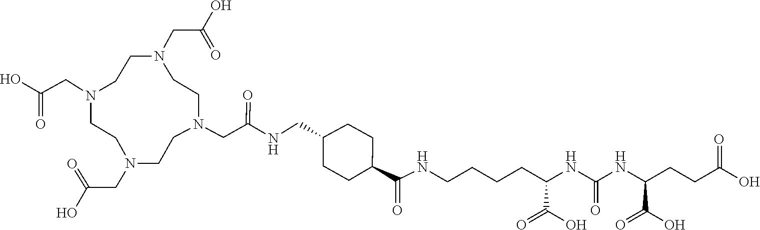 [SMILES: O=C(O)CC[C@H](NC(=O)N[C@@H](CCCCNC(=O)[C@H]1CC[C@H](CNC(=O)CN2CCN(CC(=O)O)CCN(CC(=O)O)CCN(CC(=O)O)CC2)CC1)C(=O)O)C(=O)O] |
| 60 | 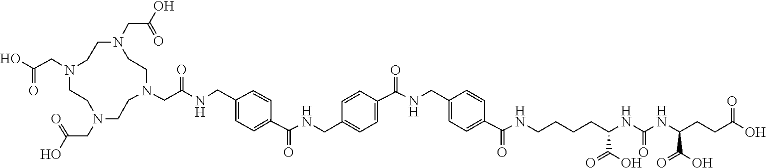 [SMILES: O=C(O)CC[C@H](NC(=O)N[C@@H](CCCCNC(=O)c1ccc(CNC(=O)c2ccc(CNC(=O)c3ccc(CNC(=O)CN4CCN(CC(=O)O)CCN(CC(=O)O)CCN(CC(=O)O)CC4)cc3)cc2)cc1)C(=O)O)C(=O)O] |
| 61 | 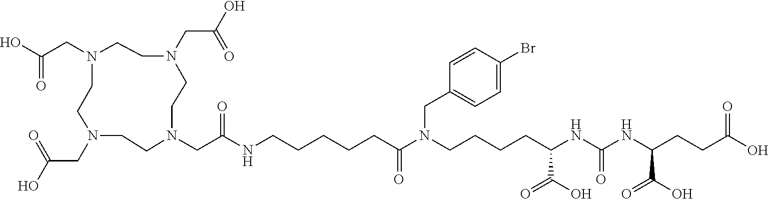 [SMILES: O=C(O)CC[C@H](NC(=O)N[C@@H](CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O] |
| 62 | 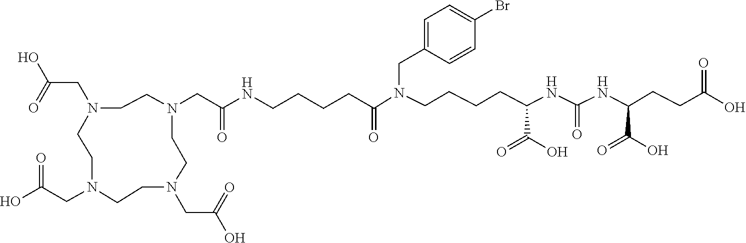 [SMILES: O=C(O)CC[C@H](NC(=O)N[C@@H](CCCCN(Cc1ccc(Br)cc1)C(=O)CCCCNC(=O)CN1CCN(CC(=O)O)CCN(CC(=O)O)CCN(CC(=O)O)CC1)C(=O)O)C(=O)O] |
| 63 | 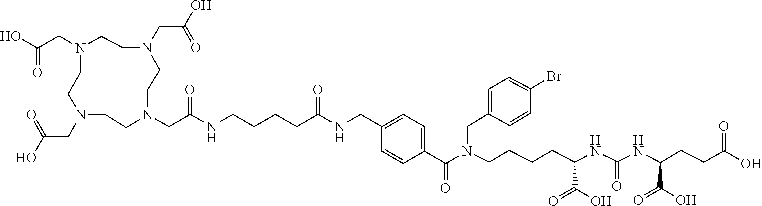 [SMILES: O=C(O)CC[C@H](NC(=O)N[C@@H](CCCCN(Cc1ccc(Br)cc1)C(=O)c1ccc(CNC(=O)CCCCNC(=O)CN2CCN(CC(=O)O)CCN(CC(=O)O)CCN(CC(=O)O)CC2)cc1)C(=O)O)C(=O)O] |
| 64 | 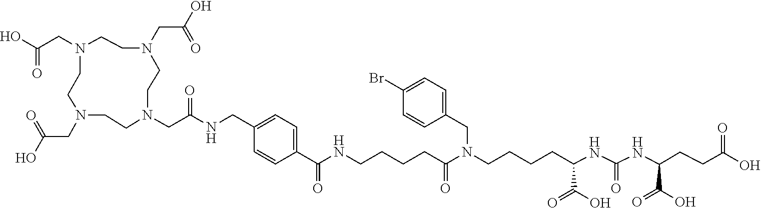 [SMILES: O=C(O)CC[C@H](NC(=O)N[C@@H](CCCCN(Cc1ccc(Br)cc1)C(=O)CCCCNC(=O)c1ccc(CNC(=O)CN2CCN(CC(=O)O)CCN(CC(=O)O)CCN(CC(=O)O)CC2)cc1)C(=O)O)C(=O)O] |
| 65 | 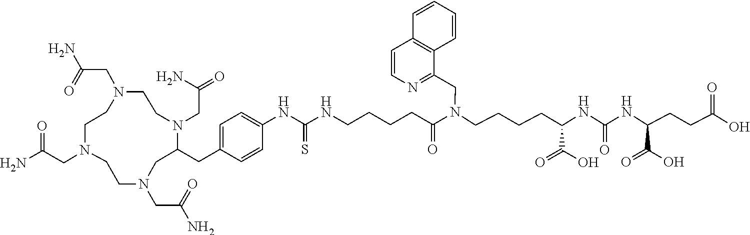 [SMILES: NC(=O)CN1CCN(CC(N)=O)CCN(CC(N)=O)C(Cc2ccc(NC(=S)NCCCCC(=O)N(CCCC[C@H](NC(=O)N[C@@H](CCC(=O)O)C(=O)O)C(=O)O)Cc3nccc4ccccc34)cc2)CN(CC(N)=O)CC1] |
| 66 | 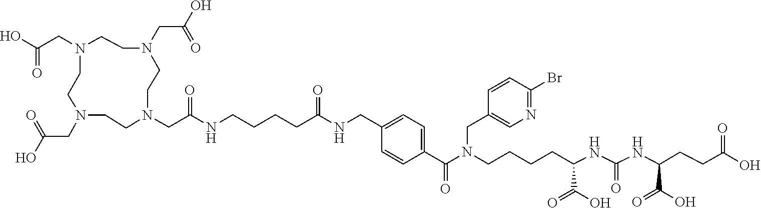 [SMILES: O=C(O)CC[C@H](NC(=O)N[C@@H](CCCCN(Cc1ccc(Br)nc1)C(=O)c1ccc(CNC(=O)CCCCNC(=O)CN2CCN(CC(=O)O)CCN(CC(=O)O)CCN(CC(=O)O)CC2)cc1)C(=O)O)C(=O)O] |
| 67 | 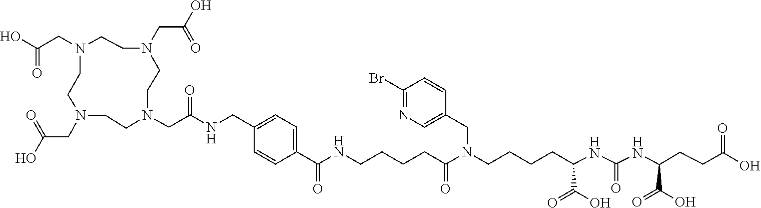 [SMILES: O=C(O)CC[C@H](NC(=O)N[C@@H](CCCCN(Cc1ccc(Br)nc1)C(=O)CCCCNC(=O)c1ccc(CNC(=O)CN2CCN(CC(=O)O)CCN(CC(=O)O)CCN(CC(=O)O)CC2)cc1)C(=O)O)C(=O)O] |
| 68 | 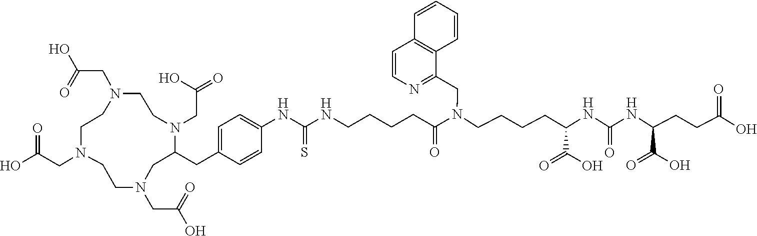 [SMILES: O=C(O)CC[C@H](NC(=O)N[C@@H](CCCCN(Cc1nccc2ccccc12)C(=O)CCCCNC(=S)Nc1ccc(CC2CN(CC(=O)O)CCN(CC(=O)O)CCN(CC(=O)O)CCN2CC(=O)O)cc1)C(=O)O)C(=O)O] |
| 69 | 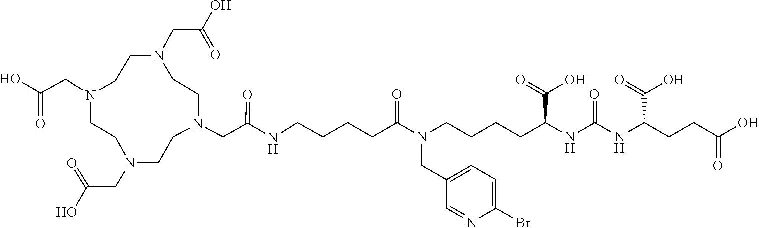 [SMILES: O=C(O)CC[C@H](NC(=O)N[C@@H](CCCCN(Cc1ccc(Br)nc1)C(=O)CCCCNC(=O)CN1CCN(CC(=O)O)CCN(CC(=O)O)CCN(CC(=O)O)CC1)C(=O)O)C(=O)O] |
| 70 | 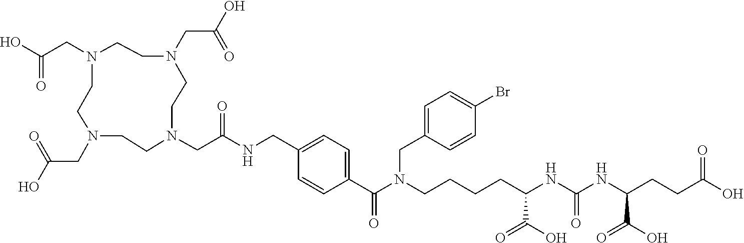 [SMILES: O=C(O)CC[C@H](NC(=O)N[C@@H](CCCCN(Cc1ccc(Br)cc1)C(=O)c1ccc(CNC(=O)CN2CCN(CC(=O)O)CCN(CC(=O)O)CCN(CC(=O)O)CC2)cc1)C(=O)O)C(=O)O] |
| 71 | 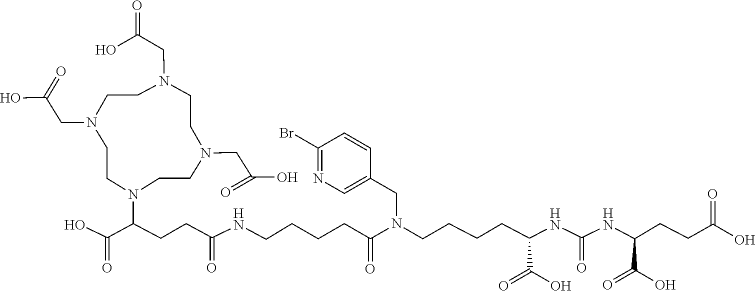 [SMILES: O=C(O)CC[C@H](NC(=O)N[C@@H](CCCCN(Cc1ccc(Br)nc1)C(=O)CCCCNC(=O)CCC(C(=O)O)N1CCN(CC(=O)O)CCN(CC(=O)O)CCN(CC(=O)O)CC1)C(=O)O)C(=O)O] |
| 72 | 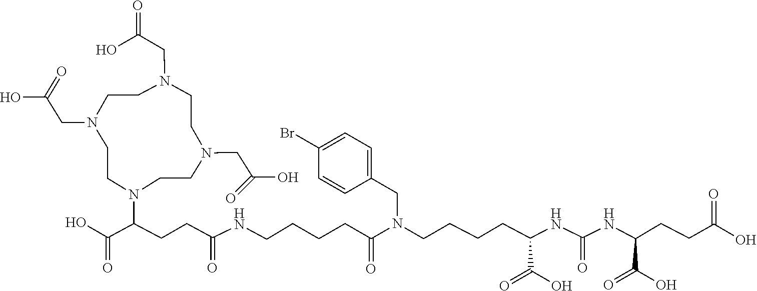 [SMILES: O=C(O)CC[C@H](NC(=O)N[C@@H](CCCCN(Cc1ccc(Br)cc1)C(=O)CCCCNC(=O)CCC(C(=O)O)N1CCN(CC(=O)O)CCN(CC(=O)O)CCN(CC(=O)O)CC1)C(=O)O)C(=O)O] |
| 73 | 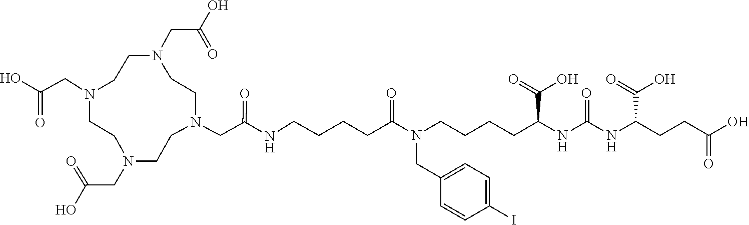 [SMILES: O=C(O)CC[C@H](NC(=O)N[C@@H](CCCCN(Cc1ccc(I)cc1)C(=O)CCCCNC(=O)CN1CCN(CC(=O)O)CCN(CC(=O)O)CCN(CC(=O)O)CC1)C(=O)O)C(=O)O] |
| 74 | 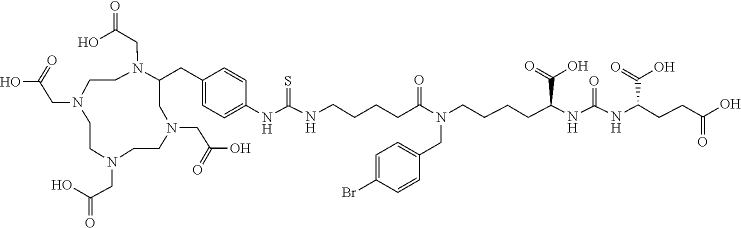 [SMILES: O=C(O)CC[C@H](NC(=O)N[C@@H](CCCCN(Cc1ccc(Br)cc1)C(=O)CCCCNC(=S)Nc1ccc(CC2CN(CC(=O)O)CCN(CC(=O)O)CCN(CC(=O)O)CCN2CC(=O)O)cc1)C(=O)O)C(=O)O] |
| 75 | 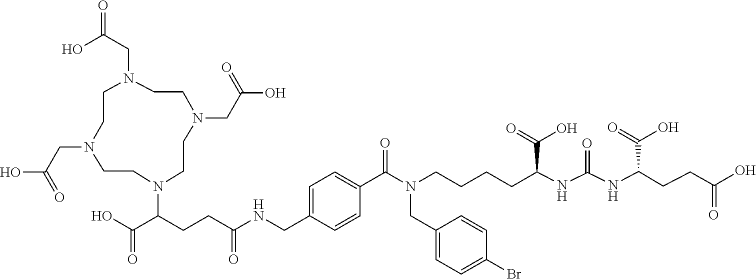 [SMILES: O=C(O)CC[C@H](NC(=O)N[C@@H](CCCCN(Cc1ccc(Br)cc1)C(=O)c1ccc(CNC(=O)CCC(C(=O)O)N2CCN(CC(=O)O)CCN(CC(=O)O)CCN(CC(=O)O)CC2)cc1)C(=O)O)C(=O)O] |
| 76 | 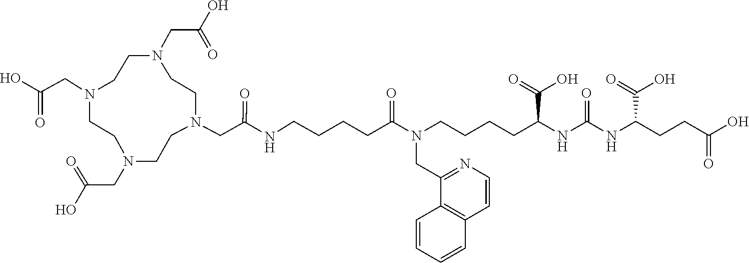 [SMILES: O=C(O)CC[C@H](NC(=O)N[C@@H](CCCCN(Cc1nccc2ccccc12)C(=O)CCCCNC(=O)CN1CCN(CC(=O)O)CCN(CC(=O)O)CCN(CC(=O)O)CC1)C(=O)O)C(=O)O] |
| 77 | 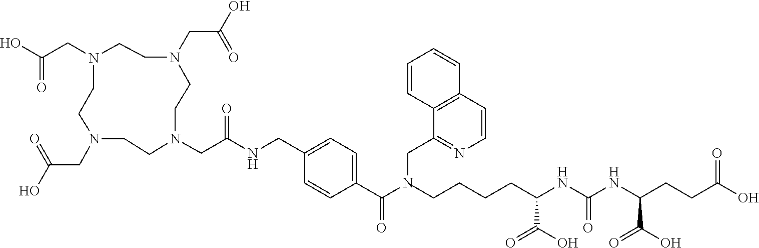 |
| 78 | 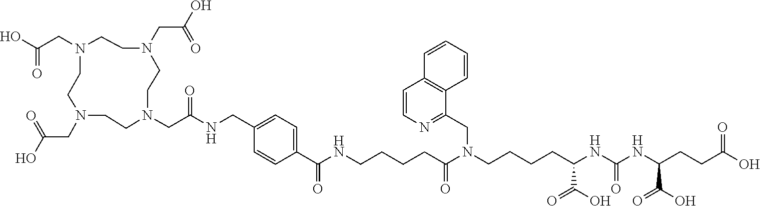 [SMILES: O=C(O)CC[C@H](NC(=O)N[C@@H](CCCCN(Cc1nccc2ccccc12)C(=O)CCCCNC(=O)c1ccc(CNC(=O)CN2CCN(CC(=O)O)CCN(CC(=O)O)CCN(CC(=O)O)CC2)cc1)C(=O)O)C(=O)O] |
| 79 | 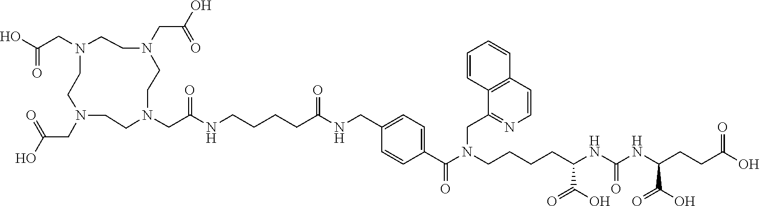 [SMILES: O=C(O)CC[C@H](NC(=O)N[C@@H](CCCCN(Cc1nccc2ccccc12)C(=O)c1ccc(CNC(=O)CCCCNC(=O)CN2CCN(CC(=O)O)CCN(CC(=O)O)CCN(CC(=O)O)CC2)cc1)C(=O)O)C(=O)O] |
| 80 | 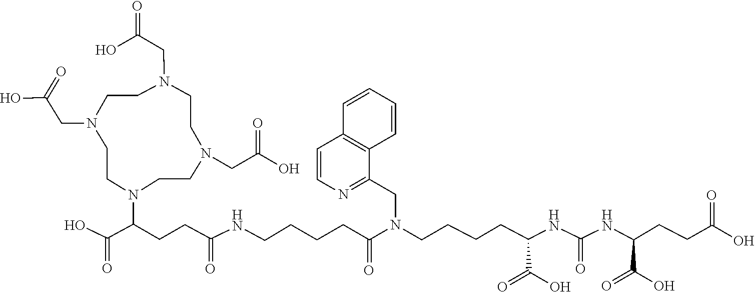 [SMILES: O=C(O)CC[C@H](NC(=O)N[C@@H](CCCCN(Cc1nccc2ccccc12)C(=O)CCCCNC(=O)CCC(C(=O)O)N1CCN(CC(=O)O)CCN(CC(=O)O)CCN(CC(=O)O)CC1)C(=O)O)C(=O)O] |
| 81 | 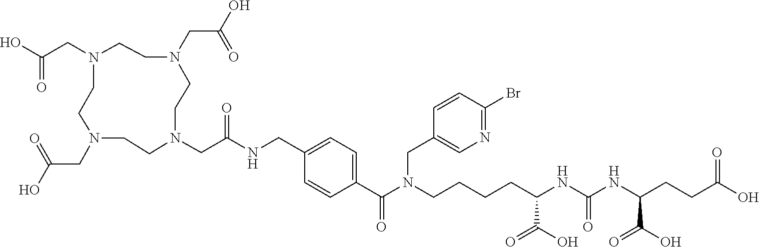 [SMILES: O=C(O)CC[C@H](NC(=O)N[C@@H](CCCCN(Cc1ccc(Br)nc1)C(=O)c1ccc(CNC(=O)CN2CCN(CC(=O)O)CCN(CC(=O)O)CCN(CC(=O)O)CC2)cc1)C(=O)O)C(=O)O] |
| 82 | 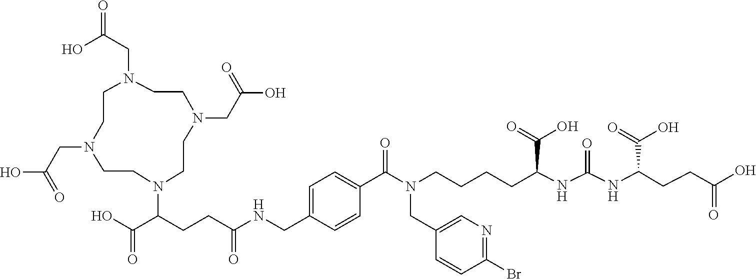 [SMILES: O=C(O)CC[C@H](NC(=O)N[C@@H](CCCCN(Cc1ccc(Br)nc1)C(=O)c1ccc(CNC(=O)CCC(C(=O)O)N2CCN(CC(=O)O)CCN(CC(=O)O)CCN(CC(=O)O)CC2)cc1)C(=O)O)C(=O)O] |
| 83 | 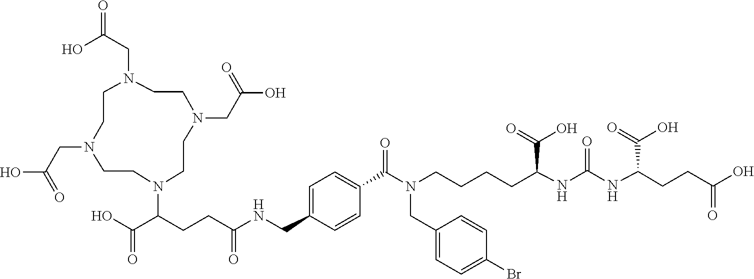 |
| 84 | 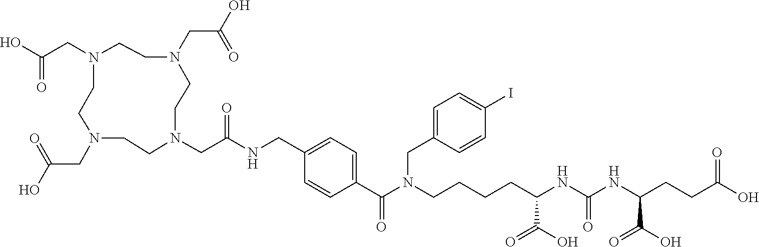 [SMILES: O=C(O)CC[C@H](NC(=O)N[C@@H](CCCCN(Cc1ccc(I)cc1)C(=O)c1ccc(CNC(=O)CN2CCN(CC(=O)O)CCN(CC(=O)O)CCN(CC(=O)O)CC2)cc1)C(=O)O)C(=O)O] |
| 85 | 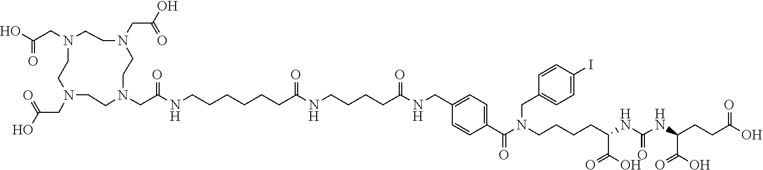 [SMILES: O=C(O)CC[C@H](NC(=O)N[C@@H](CCCCN(Cc1ccc(I)cc1)C(=O)c1ccc(CNC(=O)CCCCNC(=O)CCCCCCNC(=O)CN2CCN(CC(=O)O)CCN(CC(=O)O)CCN(CC(=O)O)CC2)cc1)C(=O)O)C(=O)O] |
| 86 | 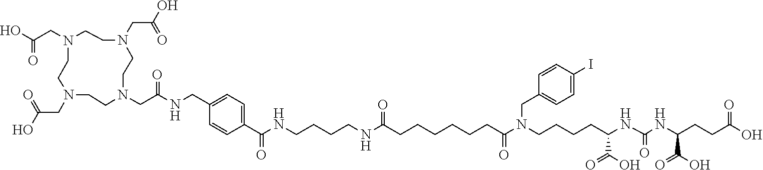 [SMILES: O=C(O)CC[C@H](NC(=O)N[C@@H](CCCCN(Cc1ccc(I)cc1)C(=O)CCCCCCC(=O)NCCCCNC(=O)c1ccc(CNC(=O)CN2CCN(CC(=O)O)CCN(CC(=O)O)CCN(CC(=O)O)CC2)cc1)C(=O)O)C(=O)O] |
| 87 | 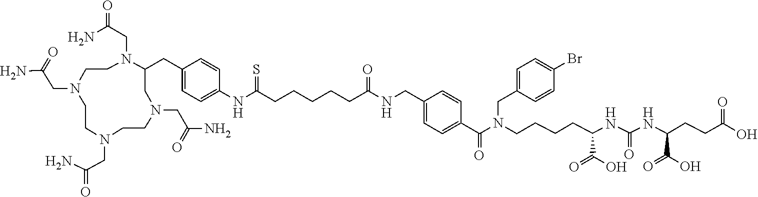 [SMILES: NC(=O)CN1CCN(CC(N)=O)CCN(CC(N)=O)C(Cc2ccc(NC(=S)CCCCCC(=O)NCc3ccc(C(=O)N(CCCC[C@H](NC(=O)N[C@@H](CCC(=O)O)C(=O)O)C(=O)O)Cc4ccc(Br)cc4)cc3)cc2)CN(CC(N)=O)CC1] |
| 88 | 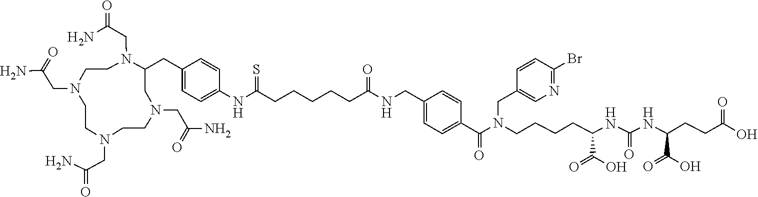 [SMILES: NC(=O)CN1CCN(CC(N)=O)CCN(CC(N)=O)C(Cc2ccc(NC(=S)CCCCCC(=O)NCc3ccc(C(=O)N(CCCC[C@H](NC(=O)N[C@@H](CCC(=O)O)C(=O)O)C(=O)O)Cc4ccc(Br)nc4)cc3)cc2)CN(CC(N)=O)CC1] |
| 89 | 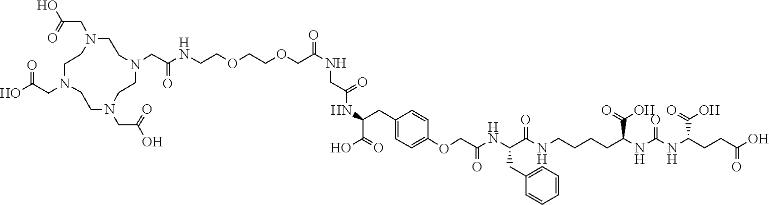 [SMILES: O=C(O)CC[C@H](NC(=O)N[C@@H](CCCCNC(=O)[C@H](Cc1ccccc1)NC(=O)COc1ccc(C[C@H](NC(=O)CNC(=O)COCCOCCNC(=O)CN2CCN(CC(=O)O)CCN(CC(=O)O)CCN(CC(=O)O)CC2)C(=O)O)cc1)C(=O)O)C(=O)O] |
| 90 | 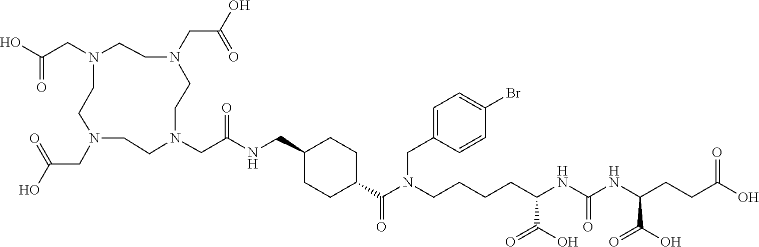 [SMILES: O=C(O)CC[C@H](NC(=O)N[C@@H](CCCCN(Cc1ccc(Br)cc1)C(=O)[C@H]1CC[C@H](CNC(=O)CN2CCN(CC(=O)O)CCN(CC(=O)O)CCN(CC(=O)O)CC2)CC1)C(=O)O)C(=O)O] |
| 91 | 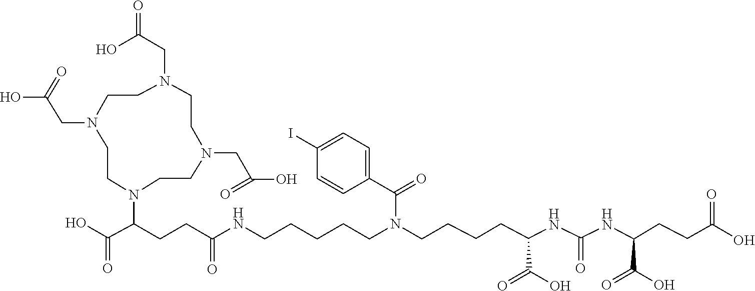 [SMILES: O=C(O)CC[C@H](NC(=O)N[C@@H](CCCCN(CCCCCNC(=O)CCC(C(=O)O)N1CCN(CC(=O)O)CCN(CC(=O)O)CCN(CC(=O)O)CC1)C(=O)c1ccc(I)cc1)C(=O)O)C(=O)O] |
| 92 | 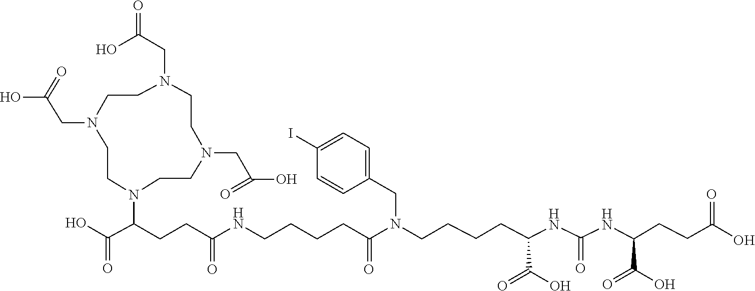 [SMILES: O=C(O)CC[C@H](NC(=O)N[C@@H](CCCCN(Cc1ccc(I)cc1)C(=O)CCCCNC(=O)CCC(C(=O)O)N1CCN(CC(=O)O)CCN(CC(=O)O)CCN(CC(=O)O)CC1)C(=O)O)C(=O)O] |
| 93 | 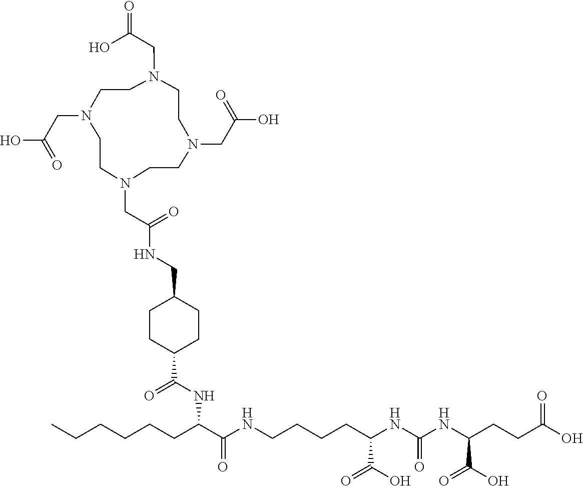 [SMILES: CCCCCC[C@H](NC(=O)[C@H]1CC[C@H](CNC(=O)CN2CCN(CC(=O)O)CCN(CC(=O)O)CCN(CC(=O)O)CC2)CC1)C(=O)NCCCC[C@H](NC(=O)N[C@@H](CCC(=O)O)C(=O)O)C(=O)O] |
| 94 | 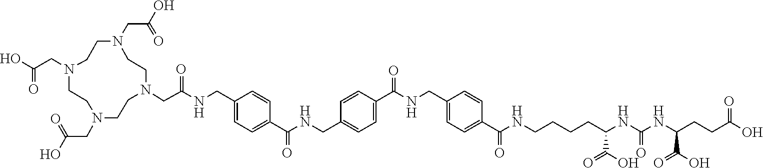 [SMILES: O=C(O)CC[C@H](NC(=O)N[C@@H](CCCCNC(=O)c1ccc(CNC(=O)c2ccc(CNC(=O)c3ccc(CNC(=O)CN4CCN(CC(=O)O)CCN(CC(=O)O)CCN(CC(=O)O)CC4)cc3)cc2)cc1)C(=O)O)C(=O)O] |
| 95 | 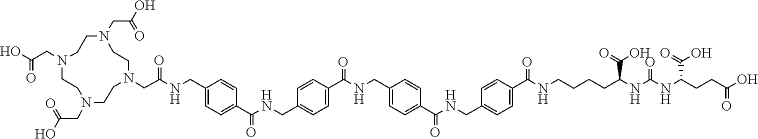 [SMILES: O=C(O)CC[C@H](NC(=O)N[C@@H](CCCCNC(=O)c1ccc(CNC(=O)c2ccc(CNC(=O)c3ccc(CNC(=O)c4ccc(CNC(=O)CN5CCN(CC(=O)O)CCN(CC(=O)O)CCN(CC(=O)O)CC5)cc4)cc3)cc2)cc1)C(=O)O)C(=O)O] |
| 96 | 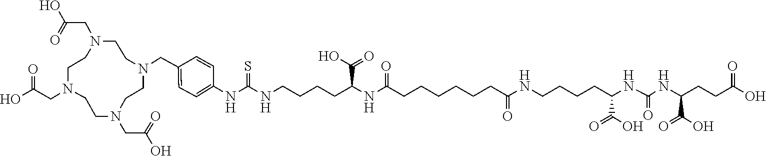 [SMILES: O=C(O)CC[C@H](NC(=O)N[C@@H](CCCCNC(=O)CCCCCCC(=O)N[C@@H](CCCCNC(=S)Nc1ccc(CN2CCN(CC(=O)O)CCN(CC(=O)O)CCN(CC(=O)O)CC2)cc1)C(=O)O)C(=O)O)C(=O)O] |
| 97 | 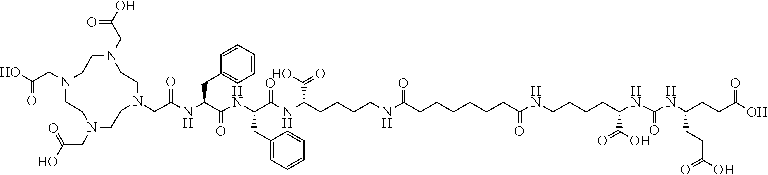 |
| 98 | 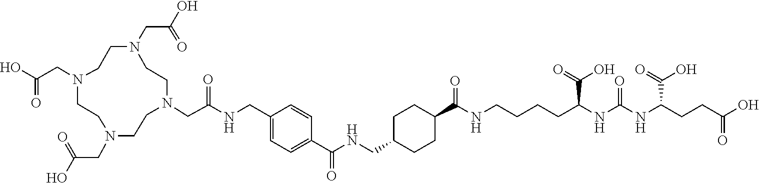 [SMILES: O=C(O)CC[C@H](NC(=O)N[C@@H](CCCCNC(=O)[C@H]1CC[C@H](CNC(=O)c2ccc(CNC(=O)CN3CCN(CC(=O)O)CCN(CC(=O)O)CCN(CC(=O)O)CC3)cc2)CC1)C(=O)O)C(=O)O] |
| 99 | 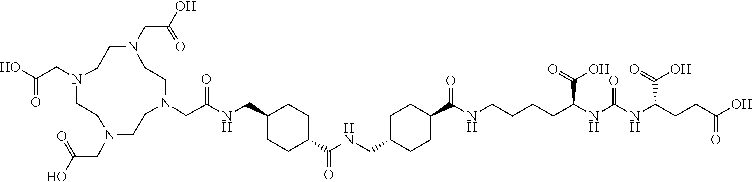 [SMILES: O=C(O)CC[C@H](NC(=O)N[C@@H](CCCCNC(=O)[C@H]1CC[C@H](CNC(=O)[C@H]2CC[C@H](CNC(=O)CN3CCN(CC(=O)O)CCN(CC(=O)O)CCN(CC(=O)O)CC3)CC2)CC1)C(=O)O)C(=O)O] |
| 100 | 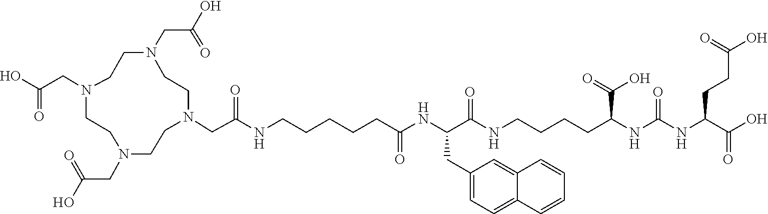 [SMILES: O=C(O)CC[C@H](NC(=O)N[C@@H](CCCCNC(=O)[C@H](Cc1ccc2ccccc2c1)NC(=O)CCCCCNC(=O)CN1CCN(CC(=O)O)CCN(CC(=O)O)CCN(CC(=O)O)CC1)C(=O)O)C(=O)O] |
| 101 | 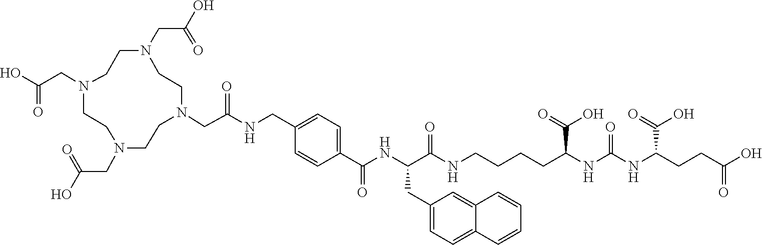 [SMILES: O=C(O)CC[C@H](NC(=O)N[C@@H](CCCCNC(=O)[C@H](Cc1ccc2ccccc2c1)NC(=O)c1ccc(CNC(=O)CN2CCN(CC(=O)O)CCN(CC(=O)O)CCN(CC(=O)O)CC2)cc1)C(=O)O)C(=O)O] |
| 102 | 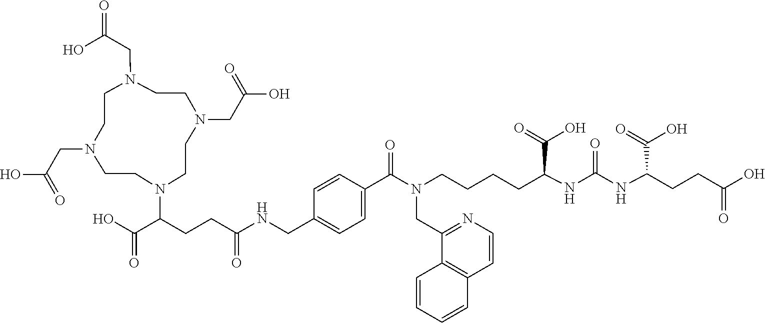 [SMILES: O=C(O)CC[C@H](NC(=O)N[C@@H](CCCCN(Cc1nccc2ccccc12)C(=O)c1ccc(CNC(=O)CCC(C(=O)O)N2CCN(CC(=O)O)CCN(CC(=O)O)CCN(CC(=O)O)CC2)cc1)C(=O)O)C(=O)O] |
| 103 | 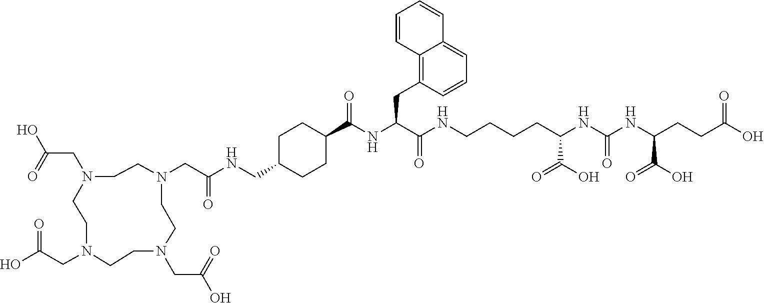 [SMILES: O=C(O)CC[C@H](NC(=O)N[C@@H](CCCCNC(=O)[C@H](Cc1cccc2ccccc12)NC(=O)[C@H]1CC[C@H](CNC(=O)CN2CCN(CC(=O)O)CCN(CC(=O)O)CCN(CC(=O)O)CC2)CC1)C(=O)O)C(=O)O] |
| 104 | 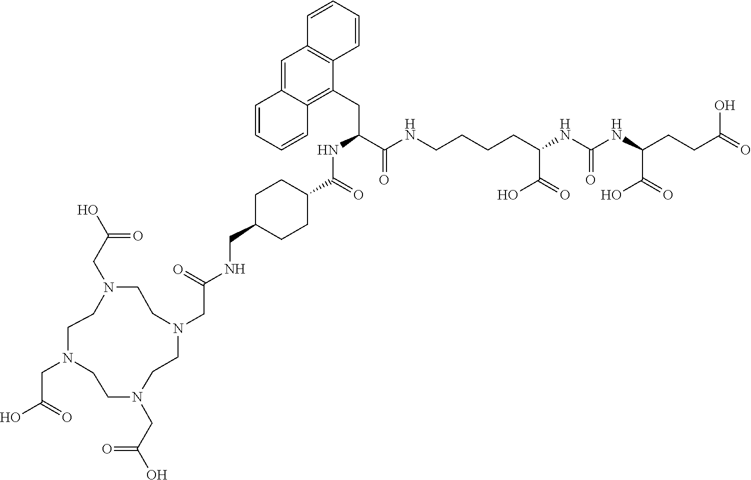 [SMILES: O=C(O)CC[C@H](NC(=O)N[C@@H](CCCCNC(=O)[C@H](Cc1c2ccccc2cc2ccccc12)NC(=O)[C@H]1CC[C@H](CNC(=O)CN2CCN(CC(=O)O)CCN(CC(=O)O)CCN(CC(=O)O)CC2)CC1)C(=O)O)C(=O)O] |
| 105 | 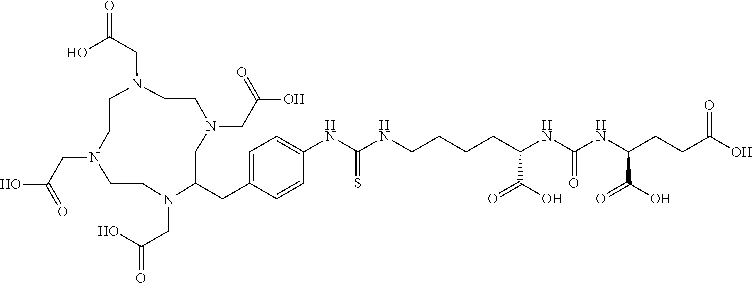 [SMILES: O=C(O)CC[C@H](NC(=O)N[C@@H](CCCCNC(=S)Nc1ccc(CC2CN(CC(=O)O)CCN(CC(=O)O)CCN(CC(=O)O)CCN2CC(=O)O)cc1)C(=O)O)C(=O)O] |
| 106 | 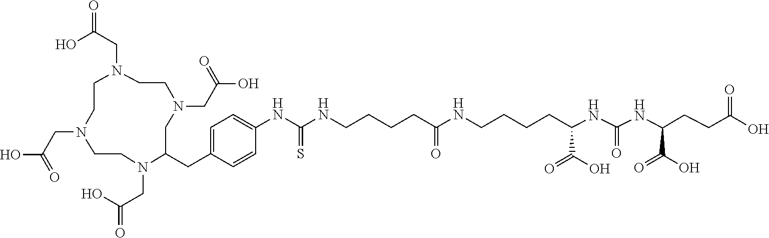 [SMILES: O=C(O)CC[C@H](NC(=O)N[C@@H](CCCCNC(=O)CCCCNC(=S)Nc1ccc(CC2CN(CC(=O)O)CCN(CC(=O)O)CCN(CC(=O)O)CCN2CC(=O)O)cc1)C(=O)O)C(=O)O] |
| 107 | 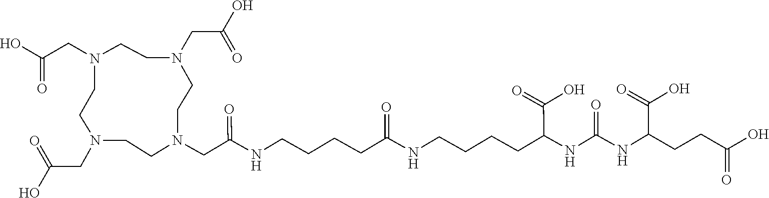 [SMILES: O=C(O)CCC(NC(=O)NC(CCCCNC(=O)CCCCNC(=O)CN1CCN(CC(=O)O)CCN(CC(=O)O)CCN(CC(=O)O)CC1)C(=O)O)C(=O)O] |
| 108 | 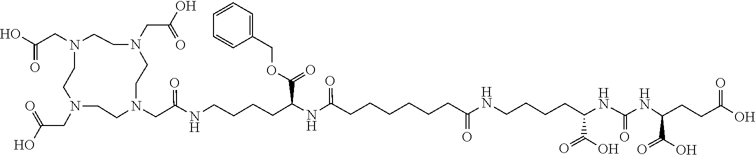 [SMILES: O=C(O)CC[C@H](NC(=O)N[C@@H](CCCCNC(=O)CCCCCCC(=O)N[C@@H](CCCCNC(=O)CN1CCN(CC(=O)O)CCN(CC(=O)O)CCN(CC(=O)O)CC1)C(=O)OCc1ccccc1)C(=O)O)C(=O)O] |
| 109 | 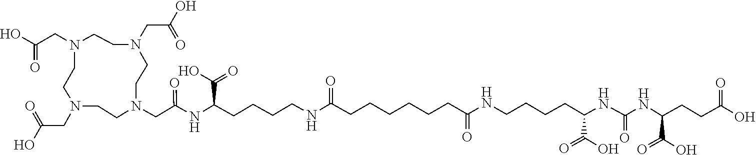 [SMILES: O=C(O)CC[C@H](NC(=O)N[C@@H](CCCCNC(=O)CCCCCCC(=O)NCCCC[C@@H](NC(=O)CN1CCN(CC(=O)O)CCN(CC(=O)O)CCN(CC(=O)O)CC1)C(=O)O)C(=O)O)C(=O)O] |
| 110 | 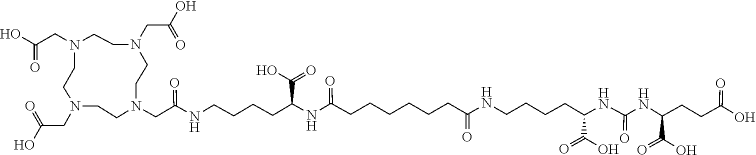 [SMILES: O=C(O)CC[C@H](NC(=O)N[C@@H](CCCCNC(=O)CCCCCCC(=O)N[C@@H](CCCCNC(=O)CN1CCN(CC(=O)O)CCN(CC(=O)O)CCN(CC(=O)O)CC1)C(=O)O)C(=O)O)C(=O)O] |
| 111 | 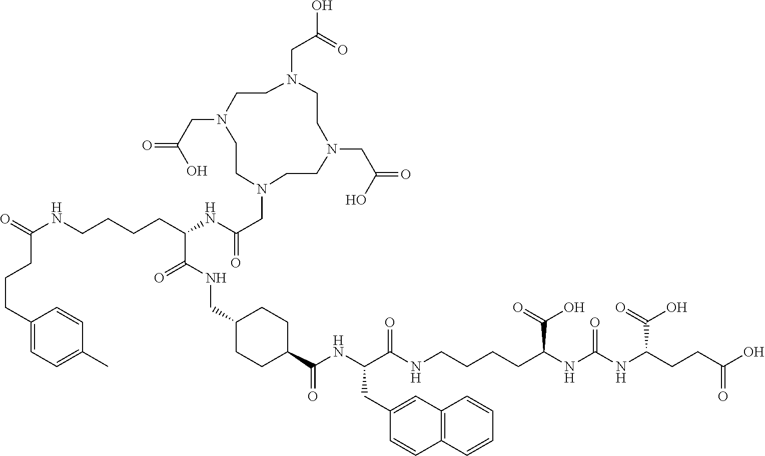 [SMILES: Cc1ccc(CCCC(=O)NCCCC[C@H](NC(=O)CN2CCN(CC(=O)O)CCN(CC(=O)O)CCN(CC(=O)O)CC2)C(=O)NC[C@H]2CC[C@H](C(=O)N[C@@H](Cc3ccc4ccccc4c3)C(=O)NCCCC[C@H](NC(=O)N[C@@H](CCC(=O)O)C(=O)O)C(=O)O)CC2)cc1] |
| 112 | 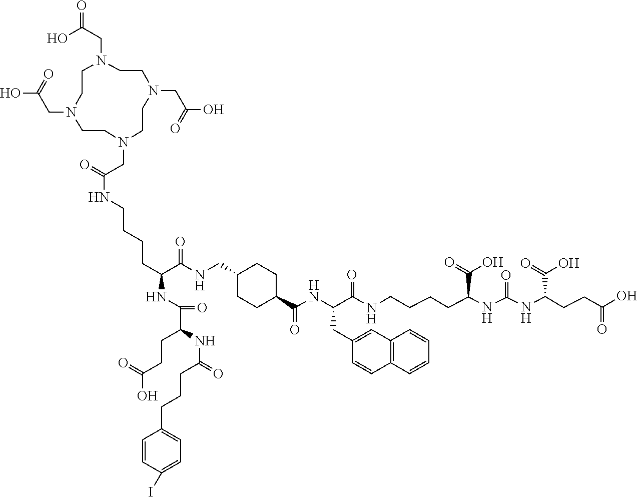 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)[C@H](CCCCNC(=O)CN2CCN(CC(=O)O)CCN(CC(=O)O)CCN(CC(=O)O)CC2)NC(=O)[C@H](CCC(=O)O)NC(=O)CCCc2ccc(I)cc2)CC1)C(=O)O)C(=O)O] |
| 113 | 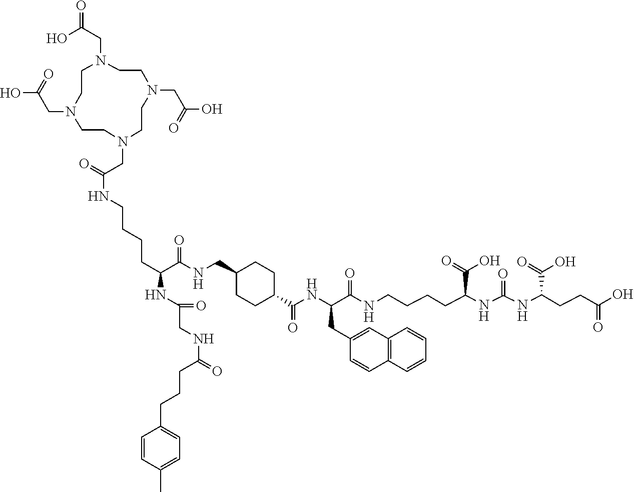 [SMILES: Cc1ccc(CCCC(=O)NCC(=O)N[C@@H](CCCCNC(=O)CN2CCN(CC(=O)O)CCN(CC(=O)O)CCN(CC(=O)O)CC2)C(=O)NC[C@H]2CC[C@H](C(=O)N[C@H](Cc3ccc4ccccc4c3)C(=O)NCCCC[C@H](NC(=O)N[C@@H](CCC(=O)O)C(=O)O)C(=O)O)CC2)cc1] |
| 114 | 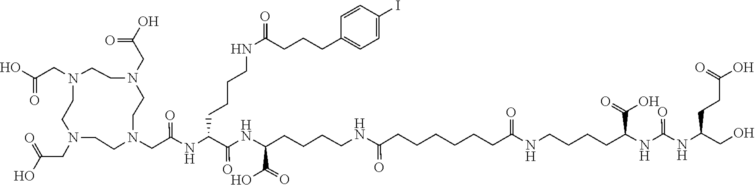 [SMILES: O=C(O)CC[C@@H](CO)NC(=O)N[C@@H](CCCCNC(=O)CCCCCCC(=O)NCCCC[C@H](NC(=O)[C@@H](CCCCNC(=O)CCCc1ccc(I)cc1)NC(=O)CN1CCN(CC(=O)O)CCN(CC(=O)O)CCN(CC(=O)O)CC1)C(=O)O)C(=O)O] |
| 115 | 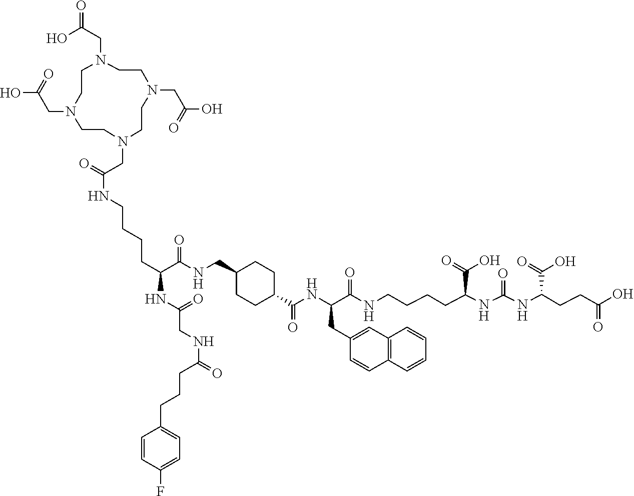 [SMILES: O=C(O)CC[C@H](NC(=O)N[C@@H](CCCCNC(=O)[C@@H](Cc1ccc2ccccc2c1)NC(=O)[C@H]1CC[C@H](CNC(=O)[C@H](CCCCNC(=O)CN2CCN(CC(=O)O)CCN(CC(=O)O)CCN(CC(=O)O)CC2)NC(=O)CNC(=O)CCCc2ccc(F)cc2)CC1)C(=O)O)C(=O)O] |
| 116 | 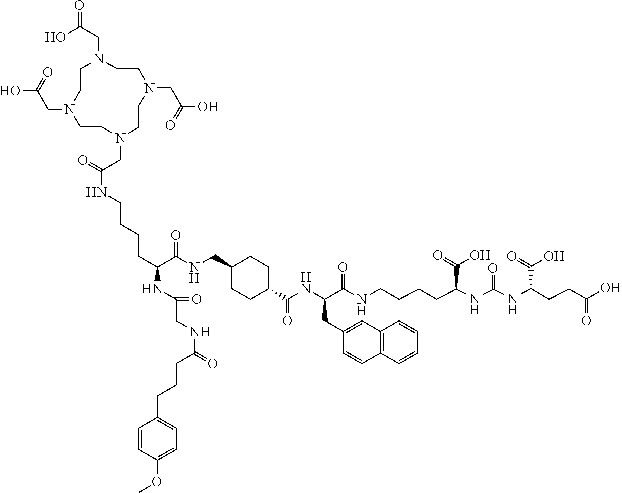 [SMILES: COc1ccc(CCCC(=O)NCC(=O)N[C@@H](CCCCNC(=O)CN2CCN(CC(=O)O)CCN(CC(=O)O)CCN(CC(=O)O)CC2)C(=O)NC[C@H]2CC[C@H](C(=O)N[C@H](Cc3ccc4ccccc4c3)C(=O)NCCCC[C@H](NC(=O)N[C@@H](CCC(=O)O)C(=O)O)C(=O)O)CC2)cc1] |
| 117 | 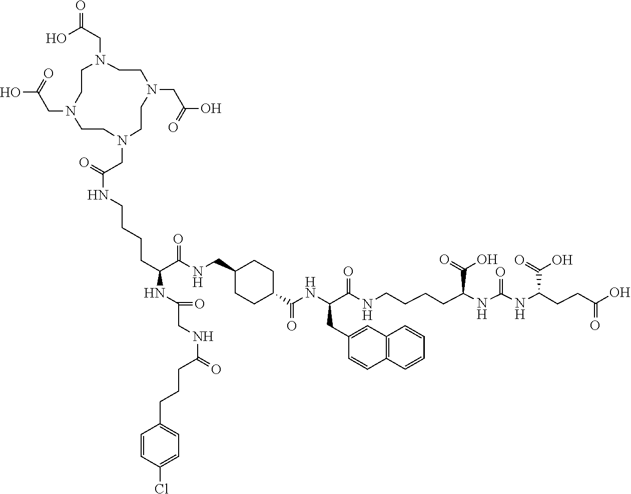 [SMILES: O=C(O)CC[C@H](NC(=O)N[C@@H](CCCCNC(=O)[C@@H](Cc1ccc2ccccc2c1)NC(=O)[C@H]1CC[C@H](CNC(=O)[C@H](CCCCNC(=O)CN2CCN(CC(=O)O)CCN(CC(=O)O)CCN(CC(=O)O)CC2)NC(=O)CNC(=O)CCCc2ccc(Cl)cc2)CC1)C(=O)O)C(=O)O] |
| 118 | 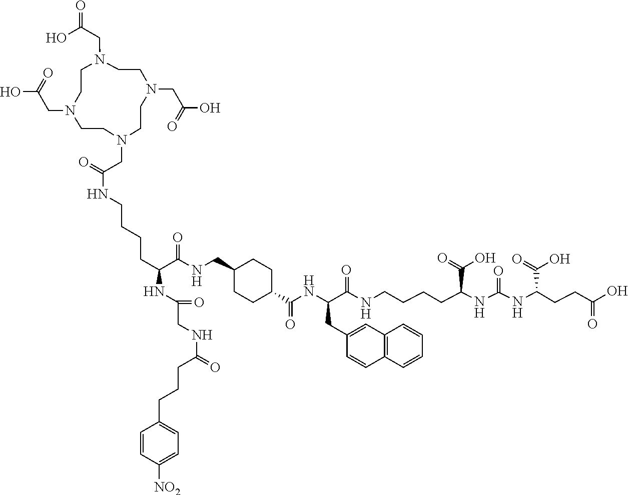 [SMILES: O=C(O)CC[C@H](NC(=O)N[C@@H](CCCCNC(=O)[C@@H](Cc1ccc2ccccc2c1)NC(=O)[C@H]1CC[C@H](CNC(=O)[C@H](CCCCNC(=O)CN2CCN(CC(=O)O)CCN(CC(=O)O)CCN(CC(=O)O)CC2)NC(=O)CNC(=O)CCCc2ccc([N+](=O)[O-])cc2)CC1)C(=O)O)C(=O)O] |
| 119 | 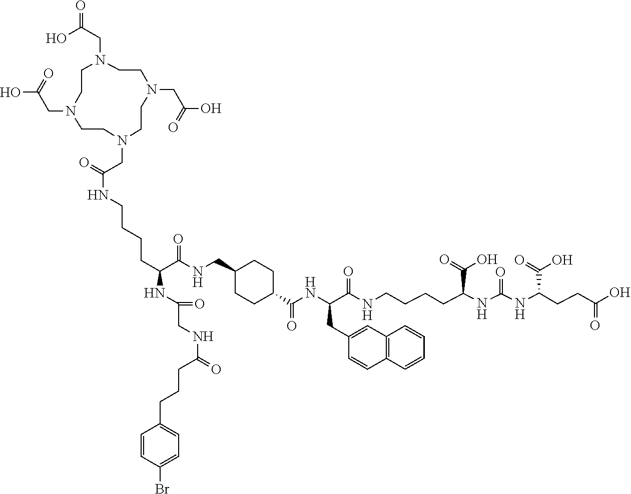 [SMILES: O=C(O)CC[C@H](NC(=O)N[C@@H](CCCCNC(=O)[C@@H](Cc1ccc2ccccc2c1)NC(=O)[C@H]1CC[C@H](CNC(=O)[C@H](CCCCNC(=O)CN2CCN(CC(=O)O)CCN(CC(=O)O)CCN(CC(=O)O)CC2)NC(=O)CNC(=O)CCCc2ccc(Br)cc2)CC1)C(=O)O)C(=O)O] |
| 120 | 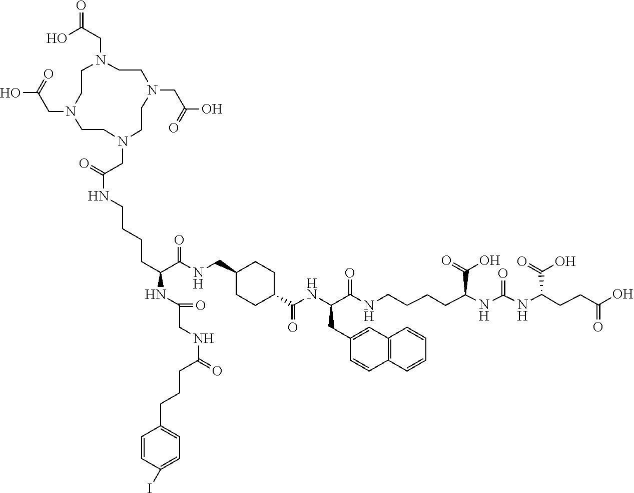 [SMILES: O=C(O)CC[C@H](NC(=O)N[C@@H](CCCCNC(=O)[C@@H](Cc1ccc2ccccc2c1)NC(=O)[C@H]1CC[C@H](CNC(=O)[C@H](CCCCNC(=O)CN2CCN(CC(=O)O)CCN(CC(=O)O)CCN(CC(=O)O)CC2)NC(=O)CNC(=O)CCCc2ccc(I)cc2)CC1)C(=O)O)C(=O)O] |
| 121 | 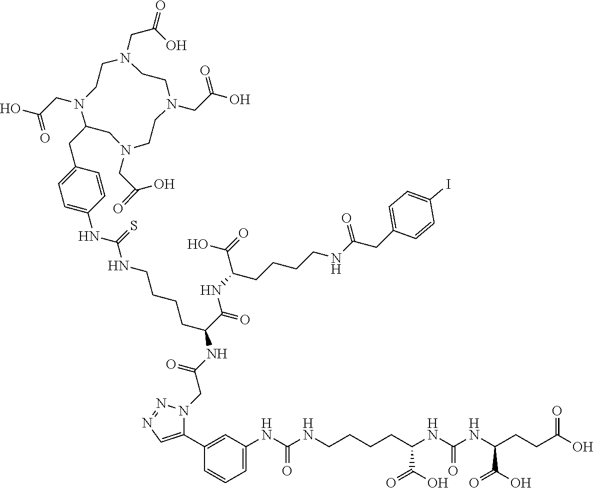 [SMILES: O=C(O)CC[C@H](NC(=O)N[C@@H](CCCCNC(=O)Nc1cccc(-c2cnnn2CC(=O)N[C@@H](CCCCNC(=S)Nc2ccc(CC3CN(CC(=O)O)CCN(CC(=O)O)CCN(CC(=O)O)CCN3CC(=O)O)cc2)C(=O)N[C@@H](CCCCNC(=O)Cc2ccc(I)cc2)C(=O)O)c1)C(=O)O)C(=O)O] |
| 122 | 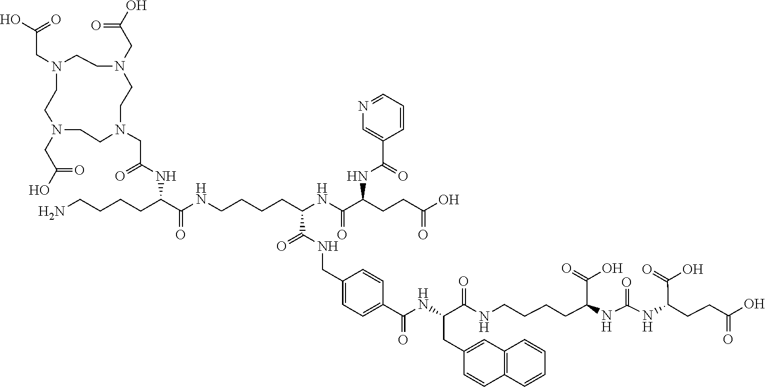 [SMILES: NCCCC[C@H](NC(=O)CN1CCN(CC(=O)O)CCN(CC(=O)O)CCN(CC(=O)O)CC1)C(=O)NCCCC[C@H](NC(=O)[C@H](CCC(=O)O)NC(=O)c1cccnc1)C(=O)NCc1ccc(C(=O)N[C@@H](Cc2ccc3ccccc3c2)C(=O)NCCCC[C@H](NC(=O)N[C@@H](CCC(=O)O)C(=O)O)C(=O)O)cc1] |
| 123 | 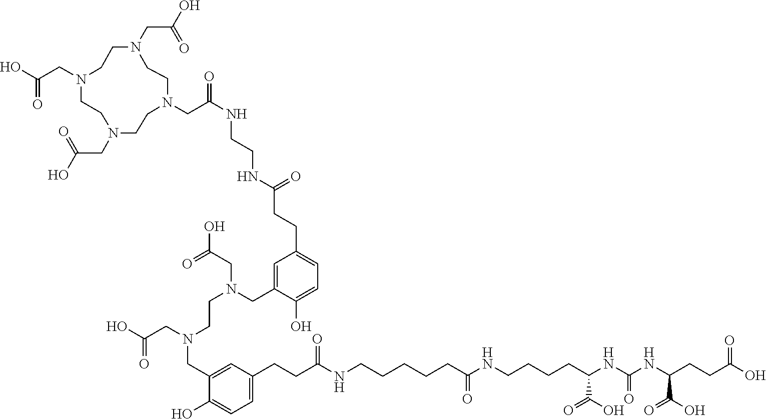 [SMILES: O=C(O)CC[C@H](NC(=O)N[C@@H](CCCCNC(=O)CCCCCNC(=O)CCc1ccc(O)c(CN(CCN(CC(=O)O)Cc2cc(CCC(=O)NCCNC(=O)CN3CCN(CC(=O)O)CCN(CC(=O)O)CCN(CC(=O)O)CC3)ccc2O)CC(=O)O)c1)C(=O)O)C(=O)O] |
| 124 | 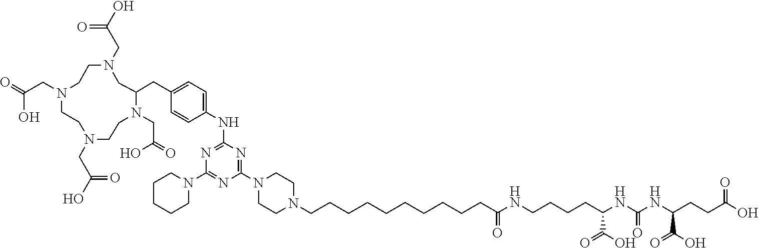 [SMILES: O=C(O)CC[C@H](NC(=O)N[C@@H](CCCCNC(=O)CCCCCCCCCCN1CCN(c2nc(Nc3ccc(CC4CN(CC(=O)O)CCN(CC(=O)O)CCN(CC(=O)O)CCN4CC(=O)O)cc3)nc(N3CCCCC3)n2)CC1)C(=O)O)C(=O)O] |
| 125 | 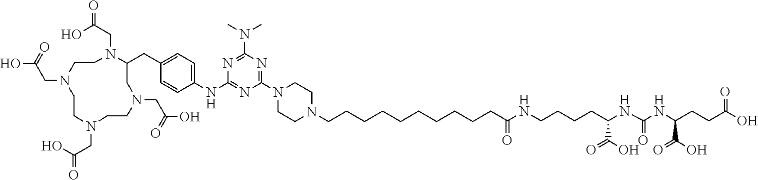 [SMILES: CN(C)c1nc(Nc2ccc(CC3CN(CC(=O)O)CCN(CC(=O)O)CCN(CC(=O)O)CCN3CC(=O)O)cc2)nc(N2CCN(CCCCCCCCCCC(=O)NCCCC[C@H](NC(=O)N[C@@H](CCC(=O)O)C(=O)O)C(=O)O)CC2)n1] |
| 126 | 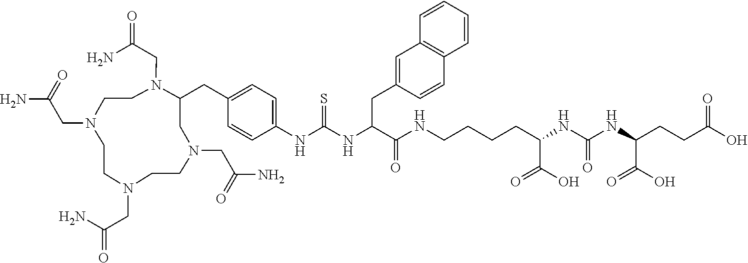 [SMILES: NC(=O)CN1CCN(CC(N)=O)CCN(CC(N)=O)C(Cc2ccc(NC(=S)NC(Cc3ccc4ccccc4c3)C(=O)NCCCC[C@H](NC(=O)N[C@@H](CCC(=O)O)C(=O)O)C(=O)O)cc2)CN(CC(N)=O)CC1] |
| 127 | 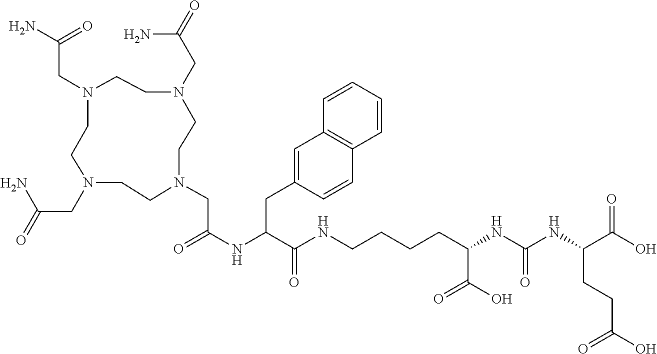 [SMILES: NC(=O)CN1CCN(CC(N)=O)CCN(CC(=O)NC(Cc2ccc3ccccc3c2)C(=O)NCCCC[C@H](NC(=O)N[C@@H](CCC(=O)O)C(=O)O)C(=O)O)CCN(CC(N)=O)CC1] |
| 128 | 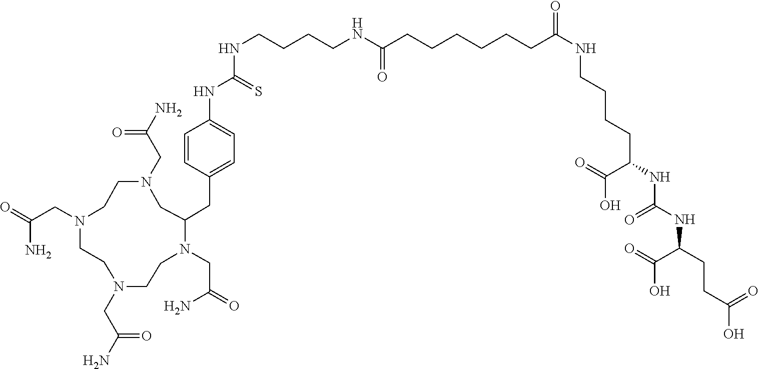 [SMILES: NC(=O)CN1CCN(CC(N)=O)CCN(CC(N)=O)C(Cc2ccc(NC(=S)NCCCCNC(=O)CCCCCCC(=O)NCCCC[C@H](NC(=O)N[C@@H](CCC(=O)O)C(=O)O)C(=O)O)cc2)CN(CC(N)=O)CC1] |
| 129 | 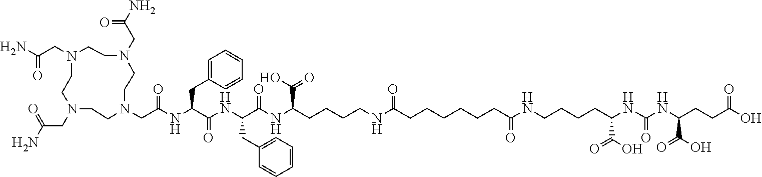 [SMILES: NC(=O)CN1CCN(CC(N)=O)CCN(CC(=O)N[C@@H](Cc2ccccc2)C(=O)N[C@@H](Cc2ccccc2)C(=O)N[C@H](CCCCNC(=O)CCCCCCC(=O)NCCCC[C@H](NC(=O)N[C@@H](CCC(=O)O)C(=O)O)C(=O)O)C(=O)O)CCN(CC(N)=O)CC1] |

In some embodiments, a compound in Table 1 comprising the structure of

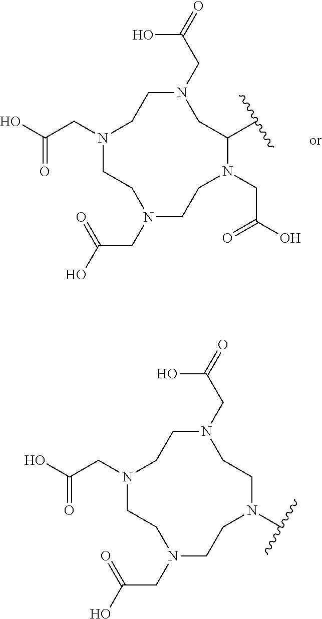 [SMILES: *C1CN(CC(=O)O)CCN(CC(=O)O)CCN(CC(=O)O)CCN1CC(=O)O ; *N1CCN(CC(=O)O)CCN(CC(=O)O)CCN(CC(=O)O)CC1]

has the structure of

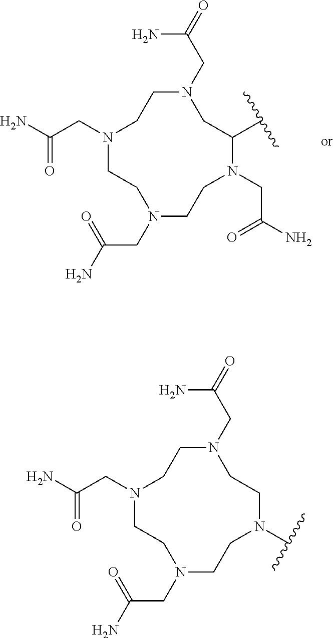 [SMILES: *C1CN(CC(N)=O)CCN(CC(N)=O)CCN(CC(N)=O)CCN1CC(N)=O ; *N1CCN(CC(N)=O)CCN(CC(N)=O)CCN(CC(N)=O)CC1]

In some embodiments, a compound in Table 1 comprises a radionuclide bound to the metal chelator, wherein the radionuclide is ²¹²Pb.

In one aspect, provided herein are radiopharmaceutical conjugates comprising a target binding moiety, a metal chelator, and a linking moiety covalently connecting the target binding moiety and the metal chelator. In some embodiments, the target binding moiety is a PSMA-binding moiety. In some embodiments, the radiopharmaceutical conjugate comprises a PSMA-binding moiety, a TCMC metal chelator, and a linking moiety covalently connecting the PSMA-binding moiety and the metal chelator. In some embodiments, the PSMA-binding moiety has the structure of

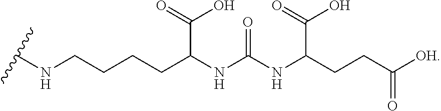 [SMILES: *NCCCCC(NC(=O)NC(CCC(=O)O)C(=O)O)C(=O)O]

In some embodiments, the linking moiety has the structure of

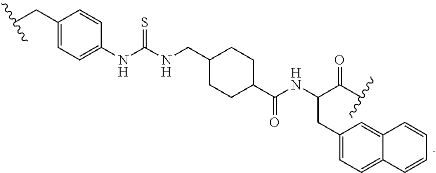 [SMILES: *Cc1ccc(NC(=S)NCC2CCC(C(=O)NC(Cc3ccc4ccccc4c3)C(*)=O)CC2)cc1]

In some embodiments, the metal chelator has the structure
of

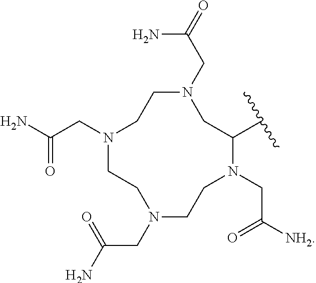 [SMILES: *C1CN(CC(N)=O)CCN(CC(N)=O)CCN(CC(N)=O)CCN1CC(N)=O]

In some embodiments, the radiopharmaceutical conjugate further comprises a radionuclide bound to the metal chelator. In some embodiments, the radiopharmaceutical conjugate comprises a compound having the structure

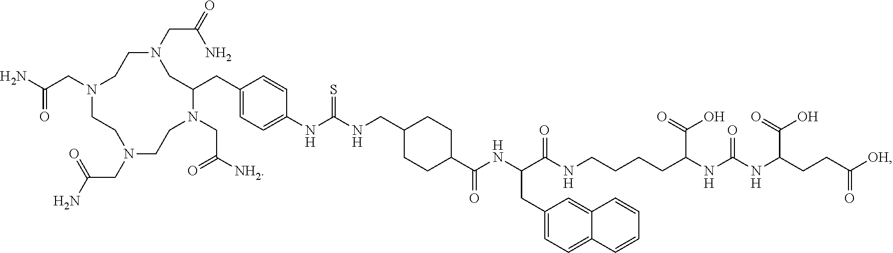 [SMILES: NC(=O)CN1CCN(CC(N)=O)CCN(CC(N)=O)C(Cc2ccc(NC(=S)NCC3CCC(C(=O)NC(Cc4ccc5ccccc5c4)C(=O)NCCCCC(NC(=O)NC(CCC(=O)O)C(=O)O)C(=O)O)CC3)cc2)CN(CC(N)=O)CC1]

or a pharmaceutically acceptable salt thereof. In some embodiments, the compound has the structure

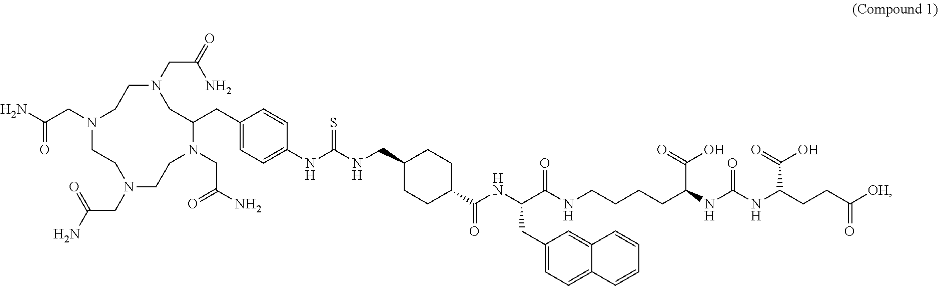

or a pharmaceutically acceptable salt thereof. In some embodiments, the radiopharmaceutical conjugate comprises a ²¹²Pb radionuclide bound to the metal chelator. In some embodiments, the radiopharmaceutical conjugate comprises a compound having the structure

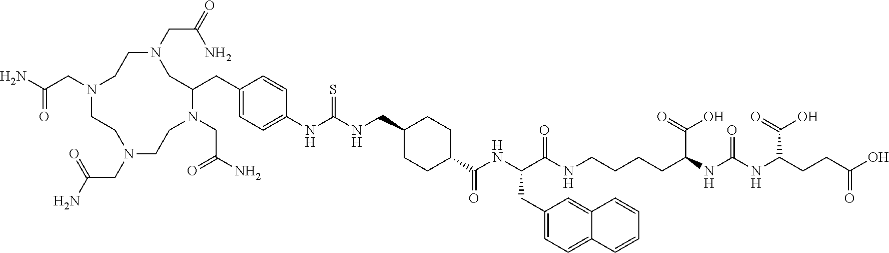

of Compound 1, and a radionuclide bound to the metal chelator, wherein the radionuclide is ²¹²Pb (²¹²Pb-Compound 1). In some embodiments, provided herein is a radiopharmaceutical conjugate having a structure of

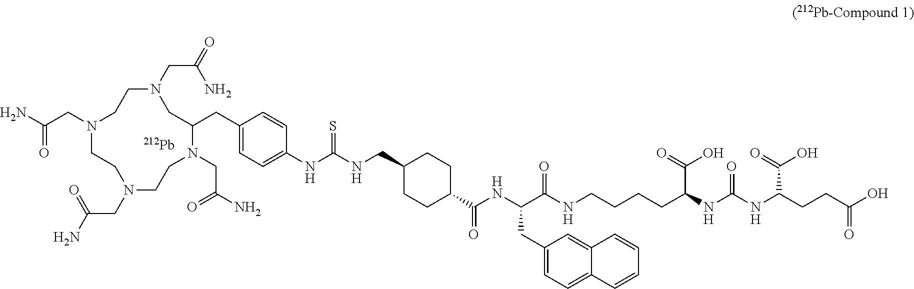

or a salt thereof.

The metal chelator, such as TCMC, can interact with the radionuclide (e.g., ²¹²Pb) via one or more functional groups and/or atoms. For example, the metal chelator can interact with the radionuclide via nitrogen and/or oxygen atoms. As another example, the metal chelator can interact with the radionuclide via amino and/or amide groups of the metal chelator. In some embodiments, the interaction of a metal chelator and a radionuclide of the conjugates disclosed herein can be illustrated as

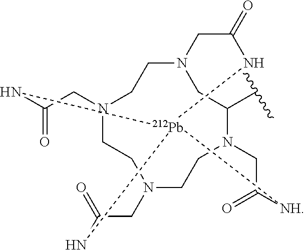

In some embodiments, the interaction of a metal chelator and a radionuclide of the conjugates disclosed herein can be illustrated as

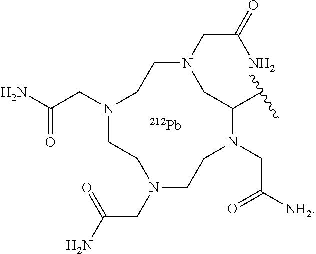

In some embodiments, ²¹²Pb-Compound 1 can be illustrated as

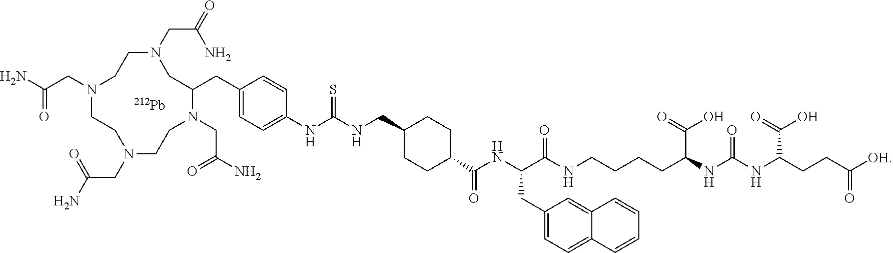

In some embodiments, the interaction of a metal chelator and a radionuclide of the conjugates disclosed herein can be illustrated as

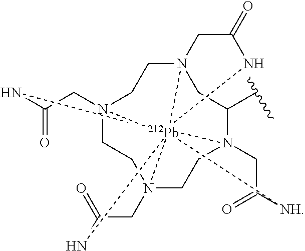

In some embodiments, the interaction of a metal chelator and a radionuclide of the conjugates disclosed herein can be illustrated as

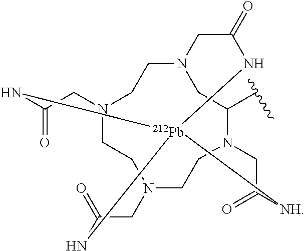

In some embodiments, the interaction of a metal chelator and a radionuclide of the conjugates disclosed herein can be illustrated as

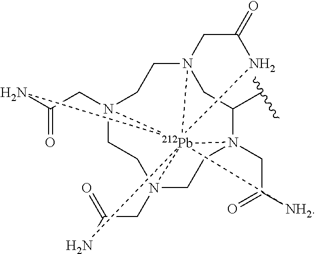

In some embodiments, ²¹²Pb-Compound 1 can be illustrated as

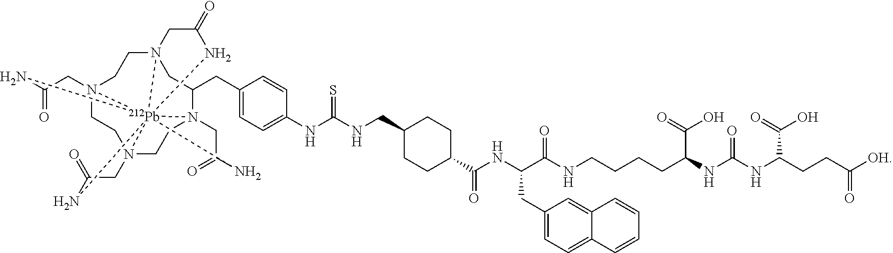

TCMC (also known as DOTAM) has a structure of

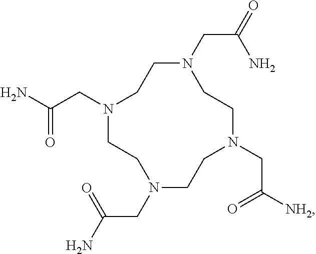

when the attachment point is not shown.

In some embodiments, Compound 1 and ²¹²Pb-Compound 1 has a reduced uptake into salivary glands. In some embodiments, the reduced salivary gland uptake of ²¹²Pb-Compound 1 lowers a risk of a subject developing xerostomia. In some embodiments, the risk of a subject developing xerostomia is reduced with ²¹²Pb-Compound 1 compared to ¹⁷⁷Lu-PSMA-617 or ²¹²Ac-PSMA-617. In some embodiments, the subject does not develop xerostomia. In some embodiments, the ²¹²Pb-Compound 1 is administered without an agent for treating xerostomia. In some embodiments, the subject is not administered an agent for treating xerostomia. In some embodiments, a subject is selected for ²¹²Pb-Compound 1 administration due to a previous xerostomia diagnosis or is susceptible of developing xerostomia. In some embodiments, the subject was diagnosed with xerostomia prior to administering ²¹²Pb-Compound 1.

### Isomers Stereoisomers

In some embodiments, the compounds described herein exist as geometric isomers. In some embodiments, the compounds described herein possess one or more double bonds. The compounds presented herein include cis, trans, syn, anti, entgegen (E), and zusammen (Z) isomers as well as the corresponding mixtures thereof. In some situations, the compounds described herein possess one or more chiral centers and each center exists in the R configuration or S configuration. The compounds described herein include diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Tautomers

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

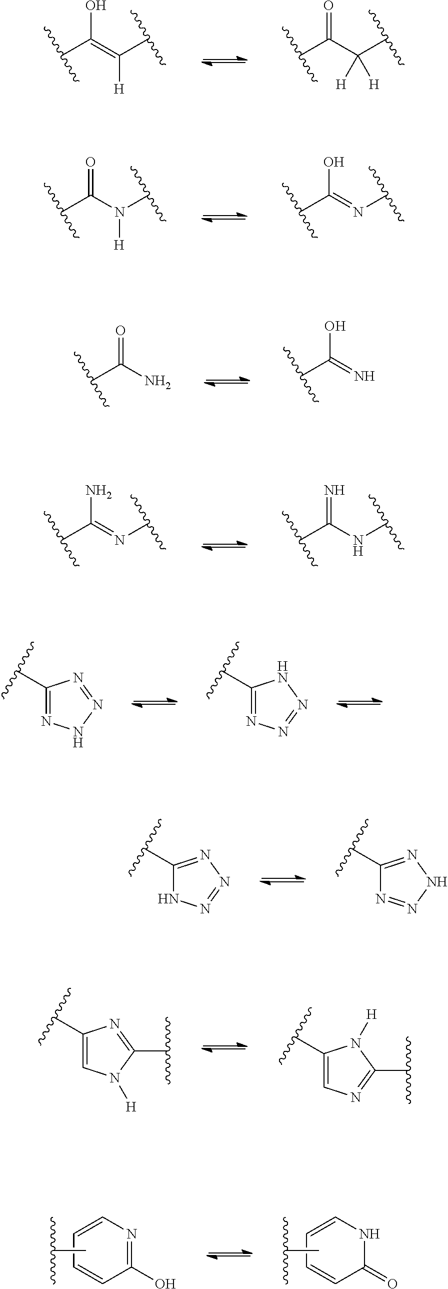

In some instances, the compounds disclosed herein exist in tautomeric forms. The structures of said compounds are illustrated in the one tautomeric form for clarity. The alternative tautomeric forms are expressly included in this disclosure.

### Labeled Compounds.

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds described herein, or a solvate, or stereoisomer thereof, include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are notable for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compound or a pharmaceutically acceptable salt, solvate, or stereoisomer thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts.

In some embodiments, the compounds described herein exist as their pharmaceutically acceptable salts. In some embodiments, the compounds disclosed herein include their pharmaceutically acceptable salts. As used herein, a “pharmaceutically acceptable salt” refers to any salt of a stabilizing agent that is useful for stabilizing the radiopharmaceutical compositions. As used herein, a “pharmaceutically acceptable salt” refers to any salt of a stabilizing agent that is useful for preventing or delaying the decomposition of the radiopharmaceutical within the compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral acid, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, y-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid, metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, the compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts, and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates.

In some embodiments, the compounds described herein exist as solvates. Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein. Accordingly, one aspect of the present disclosure pertains to hydrates and solvates of compounds of the present disclosure and/or their pharmaceutical acceptable salts, as described herein, that can be isolated and characterized by methods known in the art, such as, thermogravimetric analysis (TGA), TGA-mass spectroscopy, TGA-Infrared spectroscopy, powder X-ray diffraction (PXRD), Karl Fisher titration, high resolution X-ray diffraction, and the like.

### Pharmaceutical Compositions

The radiopharmaceutical conjugate described herein, including e.g., pharmaceutically acceptable salts or solvates thereof, can be administered as a component of a pharmaceutically acceptable formulation. In some embodiments, the radiopharmaceutical conjugate described herein is combined with a pharmaceutically suitable or acceptable carrier selected on the basis of a chosen route of administration and standard pharmaceutical practice as described, for example, in Remington: The Science and Practice of Pharmacy (Gennaro, 21^{st} Ed. Mack Pub. Co., Easton, PA (2005)). Provided herein is a radiopharmaceutical pharmaceutical composition comprising a conjugate described herein, or a stereoisomer, pharmaceutically acceptable salt, amide, ester, solvate, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition.

In one aspect, the disclosure provides a liquid radiopharmaceutical composition comprising a herein described conjugate, or a pharmaceutically acceptable salt or solvate thereof, and a pharmaceutically acceptable excipient or carrier. In some embodiments, the liquid radiopharmaceutical composition comprises
(a) a compound having the structure:

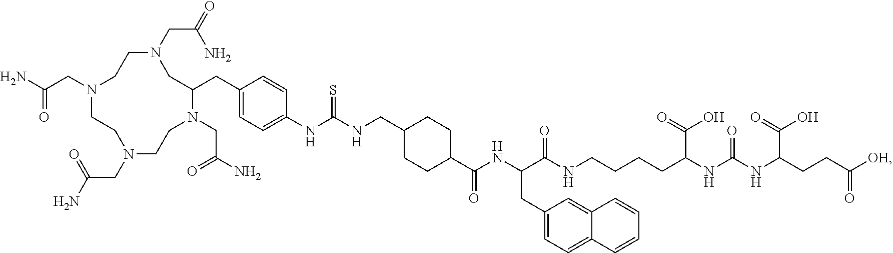 [SMILES: NC(=O)CN1CCN(CC(N)=O)CCN(CC(N)=O)C(Cc2ccc(NC(=S)NCC3CCC(C(=O)NC(Cc4ccc5ccccc5c4)C(=O)NCCCCC(NC(=O)NC(CCC(=O)O)C(=O)O)C(=O)O)CC3)cc2)CN(CC(N)=O)CC1]

or a pharmaceutically acceptable salt thereof; and
(b) a radionuclide bound to the metal chelator, wherein the radionuclide is ²¹²Pb, and wherein the liquid radiopharmaceutical composition has a radioactivity of about 75 MBq to about 300 MBq.
   In some embodiments, the liquid radiopharmaceutical composition has a radioactivity of about 75 MBq to about 200 MBq In some embodiments, the liquid radiopharmaceutical has a radioactivity of about 75 MBq. In some embodiments, the liquid radiopharmaceutical has a radioactivity of about 100 MBq. In some embodiments, wherein the liquid radiopharmaceutical has a radioactivity of about 150 MBq. In some embodiments, the liquid radiopharmaceutical has a radioactivity of about 200 MBq. In some embodiments, the liquid radiopharmaceutical has a radioactivity of about 300 MBq In some embodiments, wherein the compound is present in an amount of about 1 μg to about 1,000 μg. In some embodiments, wherein the compound is present in an amount of about 50 μg to about 500 μg. In some embodiments, wherein the compound is present in an amount of about 50 μg to about 300 μg. In some embodiments, wherein the compound is present in an amount of about 50 μg to about 150 μg. In some embodiments, wherein the compound is present in an amount of about 100 μg to about 200 μg. In some embodiments, wherein the compound is present in an amount of about 150 μg to about 250 μg. In some embodiments, wherein the compound is present in an amount of about 200 μg to about 300 μg. In some embodiments, wherein the compound is present in an amount of about 250 μg to about 350 μg. In some embodiments, wherein the compound is present in an amount of about 300 μg to about 400 μg. In some embodiments, wherein the compound is present in an amount of about 350 μg to about 450 μg. In some embodiments, wherein the compound is present in an amount of about 400 μg to about 500 μg. In some embodiments, wherein the compound is present in an amount of about 50 μg to about 100 μg. In some embodiments, wherein the compound is present in an amount of about 75 μg to about 125 μg. In some embodiments, wherein the compound is present in an amount of about 100 μg to about 150 μg. In some embodiments, wherein the compound is present in an amount of about 125 μg to about 175 μg. In some embodiments, wherein the compound is present in an amount of about 150 μg to about 200 μg. In some embodiments, wherein the compound is present in an amount of about 175 μg to about 225 μg. In some embodiments, wherein the compound is present in an amount of about 200 μg to about 250 μg. In some embodiments, wherein the compound is present in an amount of about 225 μg to about 275 μg. In some embodiments, wherein the compound is present in an amount of about 250 μg to about 300 μg.

The conjugates and pharmaceutical compositions of the current disclosure can be administered by any suitable means, e.g., by injection, including intravenous administration.

### IV. Method of Treatment

In one aspect, the disclosure provides methods of treating a disease or condition in a subject in need thereof. The methods can comprise administering a radiopharmaceutical conjugate, for example, a radiopharmaceutical of Formula (I), to the subject in need thereof. The methods can provide a therapeutic and/or prophylactic benefit to a subject in need thereof comprising administering a radiopharmaceutical conjugate described herein. In one aspect, the disclosure provides methods of preventing the formation of metastasis in a subject in need thereof.

The methods can comprise administering to a subject a therapeutically effective amount of a radiopharmaceutical conjugate or a pharmaceutically acceptable salt or solvate thereof. In some embodiments, the subject has cancer. In some embodiments, the cancer is a solid tumor. In some embodiments, the cancer is a PSMA-associated cancer. In some embodiments, the cancer is a PSMA positive cancer. In some embodiments, the cancer is prostate cancer. In some embodiments, the prostate cancer is metastatic castrate resistant prostate cancer (mCRPC). In some embodiments, the prostate cancer is progressive mCRPC. In some embodiments, provided herein is a method of treating a metastatic lesion in a subject in need thereof, comprising administering to the subject a radiopharmaceutical conjugate or a pharmaceutically acceptable salt thereof. In some embodiments, provided herein is a method of preventing or reducing the formation of a metastatic lesion in a subject in need thereof, comprising administering to the subject a radiopharmaceutical conjugate or a pharmaceutically acceptable salt thereof. In some embodiments, provided herein is a method of preventing the formation of a metastatic lesion in a subject in need thereof, comprising administering to the subject a radiopharmaceutical conjugate or a pharmaceutically acceptable salt thereof. In some embodiments, the subject has prostate cancer. In some embodiments, metastatic lesions are present in bone, viscera, lymph node(s), or a combination thereof. In some embodiments, the metastatic lesion is present in bone. In some embodiments, the metastatic lesion is present in viscera. In some embodiments, the metastatic lesion is present in a lymph node(s). In some embodiments, the metastatic lesion is present in the lung, liver, heart, or kidney. In some embodiments, the metastatic lesions have an EANM PSMA-PET score of 1, 2, or 3. In some embodiments, the EANM PSMA-PET score of the metastatic lesion is 1. In some embodiments, the EANM PSMA-PET score of the metastatic lesion is 2. In some embodiments, the EANM PSMA-PET score of the metastatic lesion is 3. In some embodiments, the subject does not have a metastatic lesion. In some embodiments, the metastatic lesion is undetectable or not detected. In some embodiments, the subject does not have a PSMA-positive metastatic lesion.

In one aspect, provided herein is a method of treating a prostate cancer in a subject in need thereof, comprising administering to the subject a radiopharmaceutical conjugate of Formula (I), Formula (I-A), Formula (I-A-1), Formula (I-B-1), or Formula (I-B-2), wherein the radiopharmaceutical conjugate is administered without an agent for treating xerostomia. In some embodiments, provided herein is a method of treating a prostate cancer in a subject in need thereof, comprising administering to the subject a radiopharmaceutical conjugate, wherein the radiopharmaceutical conjugate comprises,
(a) a compound having the structure:

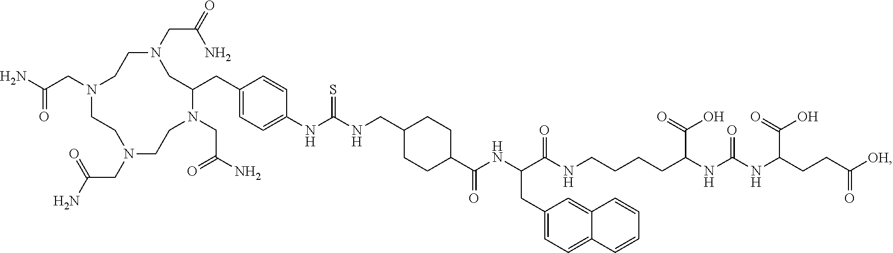 [SMILES: NC(=O)CN1CCN(CC(N)=O)CCN(CC(N)=O)C(Cc2ccc(NC(=S)NCC3CCC(C(=O)NC(Cc4ccc5ccccc5c4)C(=O)NCCCCC(NC(=O)NC(CCC(=O)O)C(=O)O)C(=O)O)CC3)cc2)CN(CC(N)=O)CC1]

or a pharmaceutically acceptable salt thereof; and
(b) a radionuclide bound to the metal chelator, wherein the radionuclide is ²¹²Pb;
   wherein the radiopharmaceutical conjugate is administered without an agent for treating xerostomia. In some embodiments, the method comprises administering a therapeutically effective amount of the radiopharmaceutical conjugate to the subject. In some embodiments, the radiopharmaceutical conjugate comprises a compound having the structure of

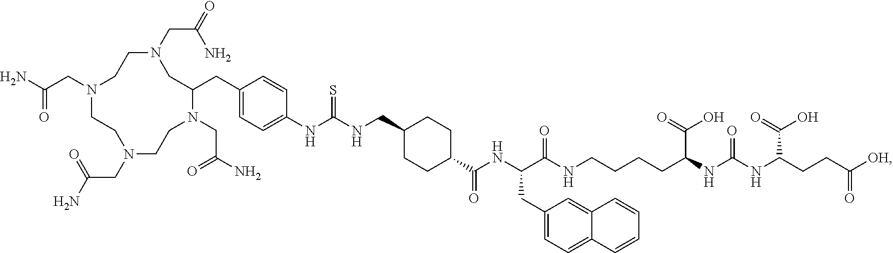

or a pharmaceutically acceptable salt thereof. In some embodiments, the radiopharmaceutical conjugate has a structure of

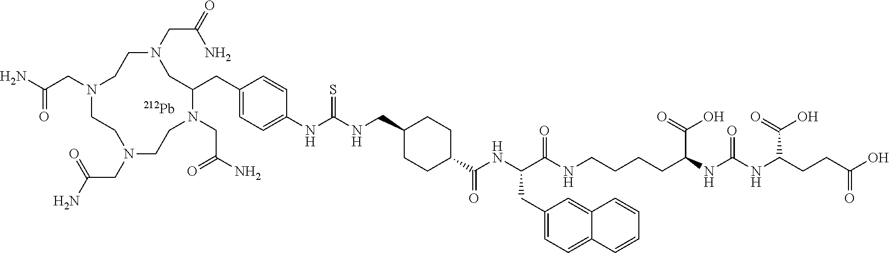

or a salt thereof. In some embodiments, the subject has previously received an anti-cancer agent before the administering of the radiopharmaceutical conjugate, and wherein the anti-cancer agent is external beam radiation, chemotherapy, hormonal therapy, or a combination thereof. In some embodiments, the anti-cancer therapy is external beam radiation. In some embodiments, the anti-cancer therapy is chemotherapy. In some embodiments, the chemotherapy is a taxane (for example, docetaxel). In some embodiments, the anti-cancer therapy is hormonal therapy (for example, bicalutamide. flutamide, and goserelin). In some embodiments, the anti-cancer therapy is an androgen receptor axis targeted agent. In some embodiments, the radiopharmaceutical conjugate is administered in combination with a second anti-cancer agent, and wherein the second anti-cancer agent is administered concurrently or sequentially with the radiopharmaceutical conjugate. In some embodiments, the second anti-cancer agent is administered sequentially (e.g., after) the radiopharmaceutical conjugate. In some embodiments, the second anti-cancer agent is administered concurrently with the radiopharmaceutical conjugate. In some embodiments, the second anti-cancer agent is administered prior to administering the radiopharmaceutical conjugate. In some embodiments, the second anti-cancer agent is an androgen receptor-axis targeted agent. In some embodiments, the second anti-cancer agent is abiraterone, enzalutamide, nilutamide, flutamide, bicalutamide, ARN 509, galeterone, orteronel, or a salt thereof. In some embodiments, the second anti-cancer agent is enzalutamide, goserelin, leuprorelin, or a salt thereof. In some embodiments, the radiopharmaceutical conjugate is administered an amount of 50 kBq to 500 MBq per dose. In some embodiments, the radiopharmaceutical conjugate is administered an amount of 100 kBq to 500 MBq per dose. In some embodiments, the radiopharmaceutical conjugate is administered in an amount of about 1 MBq to 500 MBq per dose. In some embodiments, the radiopharmaceutical conjugate is administered in an amount of about 10 MBq to 500 MBq per dose. In some embodiments, the radiopharmaceutical conjugate is administered in an amount of about 50 MBq to 500 MBq per dose. In some embodiments, the radiopharmaceutical conjugate is administered in an amount of about 75 MBq to about 300 MBq per dose. In some embodiments, the radiopharmaceutical conjugate is administered at a dose of about 10 MBq. In some embodiments, the radiopharmaceutical conjugate is administered at a dose of about 100 MBq. In some embodiments, the agent for treating xerostomia includes agents for treating dry mouth, aptyalism, or dry throat (e.g., spray substitution of saliva). In some embodiments, the subject is not administered the agent for treating xerostomia. In some embodiments, the subject is not administered an agent for treating xerostomia for a period of 3 months, 6 months, 9 months, or 12 months since the administering of the radiopharmaceutical conjugate. In some embodiments, the subject is not administered an agent for treating xerostomia for a period of 3 months, 6 months, 9 months, or 12 months prior to the administering of the radiopharmaceutical conjugate. In some embodiments, the subject is not administered an agent for treating xerostomia for a period of at least 3 months, at least 6 months, at least 9 months, or at least 12 months before and after the administering of the radiopharmaceutical conjugate. In some embodiments, the subject is not administered an agent for treating xerostomia for a period of at least 4 weeks, at least 8 weeks, at least 12 weeks, or at least 20 weeks before and after the administering of the radiopharmaceutical conjugate. In some embodiments, administering the radiopharmaceutical conjugate reduces a risk of xerostomia in the subject, optionally wherein the risk is reduced relative to the administering of ¹⁷⁷Lu-PSMA-617 or ²²⁵Ac-PSMA-617. In some embodiments, the risk is relative to the administering of ²²⁵Ac-PSMA-617. In some embodiments, the subject has received a targeted radiopharmaceutical therapy prior to administering of the radiopharmaceutical conjugate. In some embodiments, the targeted radiopharmaceutical therapy comprises ¹⁷⁷Lu-PSMA-617 or ²²⁵Ac-PSMA-617. In some embodiments, the targeted radiopharmaceutical therapy comprises ¹⁷⁷Lu-PSMA-617. In some embodiments, the subject has progressive prostate cancer after prior treatment with ¹⁷⁷Lu-PSMA-617 or ²²⁵Ac-PSMA-617. In some embodiments, the subject is fasted prior to administering of the radiopharmaceutical conjugate. In some embodiments, the administering of the radiopharmaceutical conjugate does not cause or increase the risk of xerostomia in the subject. In some embodiments, the radiopharmaceutical conjugate is not significantly taken up in a salivary gland in the subject. In some embodiments, the subject does not develop xerostomia after the administering of the radiopharmaceutical conjugate. In some embodiments, the subject was previously diagnosed with xerostomia prior to administering the radiopharmaceutical conjugate. In some embodiments, the subject is susceptible of developing xerostomia. In some embodiments, the subject has an increased risk of developing xerostomia. In some embodiments, the subject is diagnosed as having an increased risk of developing xerostomia. In some embodiments, the administering does not cause any one or more of xerostomia, radiation nephropathy, or bone marrow toxicity, or hematological toxicity in the subject. In some embodiments, the prostate cancer is metastatic castrate resistant prostate cancer (mCRPC). In some embodiments, the prostate cancer is progressive mCRPC. In some embodiments, administering the radiopharmaceutical conjugate prevents or reduces the formation of a metastatic lesion in the subject. In some embodiments, the metastatic lesion comprises a metastatic lesion in bone, viscera, lymph node(s), or a combination thereof. In some embodiments, the metastatic lesion is a metastatic bone lesion. In some embodiments, the metastatic lesion is present in the subject prior to administering the radiopharmaceutical conjugate, and administering the radiopharmaceutical conjugate treats, reduces, or eliminates the metastatic lesion. In some embodiments, the administering prevents or reduces metastasis formation. In some embodiments, the subject does not have a metastatic lesion (e.g., metastatic lesions in bone, viscera, lymph node(s), or a combination thereof) prior to administering the radiopharmaceutical conjugate, and the administering prevents or reduces formation of the metastatic lesions in the subject. In some embodiments, the prostate cancer is metastatic and comprises skeletal metastases prior to administering the radiopharmaceutical conjugate to the subject, and the administering prevents or reduces further skeletal metastases. In some embodiments, the prostate cancer is local and administering the radiopharmaceutical conjugate to the subject prevents or reduces skeletal metastases. In some embodiments, the prostate cancer is metastatic, but does not comprise bone lesions, and administering the radiopharmaceutical conjugate to the subject prevents or reduces further skeletal metastases. In some embodiments, the radiopharmaceutical conjugate is administered via intravenous injection. In some embodiments, the radiopharmaceutical conjugate is administered via bolus intravenous injection.

In another aspect, provided herein is a method of treating a prostate cancer in a subject in need thereof, comprising administering to the subject a radiopharmaceutical conjugate of Formula (I), Formula (I-A), Formula (I-A-1), Formula (I-B-1), or Formula (I-B-2), wherein the subject has previously received an anti-cancer agent before the administering of the radiopharmaceutical conjugate. In some embodiments, provided herein is a method of treating a prostate cancer in a subject in need thereof, comprising administering to the subject a radiopharmaceutical conjugate, wherein the radiopharmaceutical conjugate comprises,
(a) a compound having the structure:

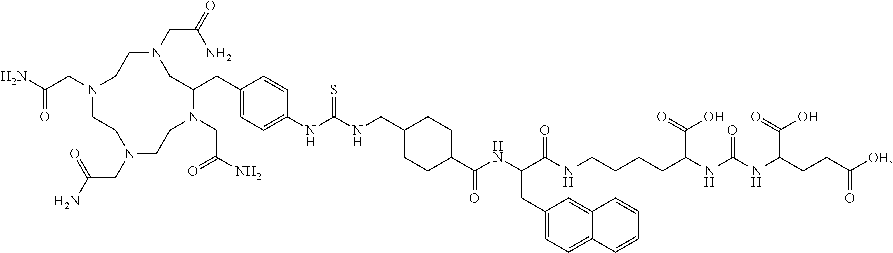 [SMILES: NC(=O)CN1CCN(CC(N)=O)CCN(CC(N)=O)C(Cc2ccc(NC(=S)NCC3CCC(C(=O)NC(Cc4ccc5ccccc5c4)C(=O)NCCCCC(NC(=O)NC(CCC(=O)O)C(=O)O)C(=O)O)CC3)cc2)CN(CC(N)=O)CC1]

or a pharmaceutically acceptable salt thereof; and
(b) a radionuclide bound to the metal chelator, wherein the radionuclide is ²¹²Pb;
   wherein the subject has previously received an anti-cancer agent before the administering of the radiopharmaceutical conjugate. In some embodiments, the method comprises administering a therapeutically effective amount of the radiopharmaceutical conjugate to the subject. In some embodiments, the radiopharmaceutical conjugate comprises a compound having the structure of

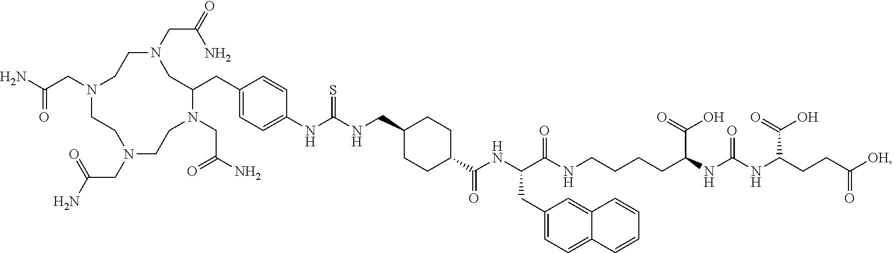

or a pharmaceutically acceptable salt thereof. In some embodiments, the anti-cancer agent is external beam radiation, chemotherapy, hormonal therapy, or a combination thereof. In some embodiments, the anti-cancer therapy is external beam radiation. In some embodiments, the anti-cancer therapy is chemotherapy. In some embodiments, the chemotherapy is a taxane (for example, docetaxel). In some embodiments, the anti-cancer therapy is hormonal therapy (for example, bicalutamide. flutamide, and goserelin). In some embodiments, the anti-cancer therapy is an androgen receptor axis targeted agent. In some embodiments, the radiopharmaceutical conjugate is administered in combination with a second anti-cancer agent, and wherein the second anti-cancer agent is administered concurrently or sequentially with the radiopharmaceutical conjugate. In some embodiments, the second anti-cancer agent is administered sequentially (e.g., after) the radiopharmaceutical conjugate. In some embodiments, the second anti-cancer agent is administered concurrently with the radiopharmaceutical conjugate. In some embodiments, the second anti-cancer agent is administered prior to administering the radiopharmaceutical conjugate. In some embodiments, the second anti-cancer agent is an androgen receptor-axis targeted agent. In some embodiments, the second anti-cancer agent is abiraterone, enzalutamide, nilutamide, flutamide, bicalutamide, ARN 509, galeterone, orteronel, or a salt thereof. In some embodiments, the second anti-cancer agent is enzalutamide, goserelin, leuprorelin, or a salt thereof. In some embodiments, the radiopharmaceutical conjugate is administered an amount of 50 kBq to 500 MBq per dose. In some embodiments, the radiopharmaceutical conjugate is administered an amount of 100 kBq to 500 MBq per dose. In some embodiments, the radiopharmaceutical conjugate is administered in an amount of about 1 MBq to 500 MBq per dose. In some embodiments, the radiopharmaceutical conjugate is administered in an amount of about 10 MBq to 500 MBq per dose. In some embodiments, the radiopharmaceutical conjugate is administered in an amount of about 50 MBq to 500 MBq per dose. In some embodiments, the radiopharmaceutical conjugate is administered in an amount of about 75 MBq to about 300 MBq per dose. In some embodiments, the radiopharmaceutical conjugate is administered at a dose of about 10 MBq. In some embodiments, the radiopharmaceutical conjugate is administered at a dose of about 100 MBq. In some embodiments, the radiopharmaceutical conjugate is administered without an agent for treating xerostomia. In some embodiments, the agent for treating xerostomia includes agents for treating dry mouth, aptyalism, or dry throat (e.g., spray substitution of saliva). In some embodiments, the subject is not administered the agent for treating xerostomia. In some embodiments, administering the radiopharmaceutical conjugate reduces a risk of xerostomia in the subject, optionally wherein the risk is reduced relative to the administering of ¹⁷⁷Lu-PSMA-617 or ²²⁵Ac-PSMA-617. In some embodiments, the risk is relative to the administering of ²²⁵Ac-PSMA-617. In some embodiments, the subject has received a targeted radiopharmaceutical therapy prior to administering of the radiopharmaceutical conjugate. In some embodiments, the targeted radiopharmaceutical therapy comprises ¹⁷⁷Lu-PSMA-617 or ²²⁵Ac-PSMA-617. In some embodiments, the targeted radiopharmaceutical therapy comprises ¹⁷⁷Lu-PSMA-617. In some embodiments, the subject has progressive prostate cancer after prior treatment with ¹⁷⁷Lu-PSMA-617 or ²²⁵Ac-PSMA-617. In some embodiments, the subject is fasted prior to administering of the radiopharmaceutical conjugate. In some embodiments, the administering of the radiopharmaceutical conjugate does not cause or increase the risk of xerostomia in the subject. In some embodiments, the radiopharmaceutical conjugate is not significantly taken up in a salivary gland in the subject. In some embodiments, the subject does not develop xerostomia after the administering of the radiopharmaceutical conjugate. In some embodiments, the subject was previously diagnosed with xerostomia prior to administering the radiopharmaceutical conjugate. In some embodiments, the subject is susceptible of developing xerostomia. In some embodiments, the administering does not cause any one or more of xerostomia, radiation nephropathy, or bone marrow toxicity, or hematological toxicity in the subject. In some embodiments, the prostate cancer is metastatic castrate resistant prostate cancer (mCRPC). In some embodiments, the prostate cancer is progressive mCRPC. In some embodiments, administering the radiopharmaceutical conjugate prevents or reduces the formation of a metastatic lesion in the subject. In some embodiments, the metastatic lesion comprises a metastatic lesion in bone, viscera, lymph node(s), or a combination thereof. In some embodiments, the metastatic lesion is a metastatic bone lesion. In some embodiments, the metastatic lesion is present in the subject prior to administering the radiopharmaceutical conjugate, and administering the radiopharmaceutical conjugate treats, reduces, or eliminates the metastatic lesion. In some embodiments, the administering prevents or reduces metastasis formation. In some embodiments, the subject does not have a metastatic lesion (e.g., metastatic lesions in bone, viscera, lymph node(s), or a combination thereof) prior to administering the radiopharmaceutical conjugate, and the administering prevents or reduces formation of the metastatic lesions in the subject. In some embodiments, the prostate cancer is metastatic and comprises skeletal metastases prior to administering the radiopharmaceutical conjugate to the subject, and the administering prevents or reduces further skeletal metastases. In some embodiments, the prostate cancer is local and administering the radiopharmaceutical conjugate to the subject prevents or reduces skeletal metastases. In some embodiments, the prostate cancer is metastatic, but does not comprise bone lesions, and administering the radiopharmaceutical conjugate to the subject prevents or reduces further skeletal metastases. In some embodiments, the radiopharmaceutical conjugate is administered via intravenous injection. In some embodiments, the radiopharmaceutical conjugate is administered via bolus intravenous injection.

In another aspect, provided herein is a method of treating a metastatic prostate cancer in a subject in need thereof, comprising administering to the subject a radiopharmaceutical conjugate of Formula (I), Formula (I-A), Formula (I-A-1), Formula (I-B-1), or Formula (I-B-2). In some embodiments, the method of treating the metastatic prostate cancer in a subject in need thereof comprises administering to the subject a radiopharmaceutical conjugate comprising:
(a) a compound having the structure:

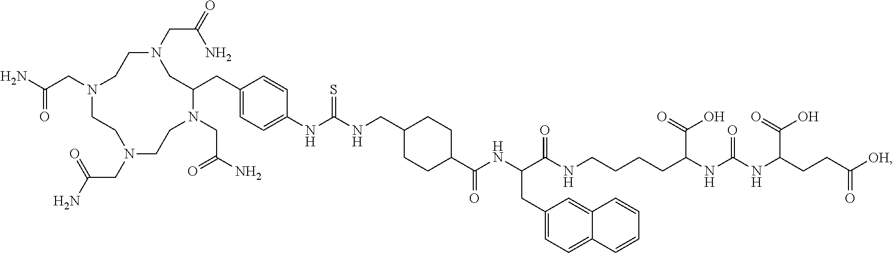 [SMILES: NC(=O)CN1CCN(CC(N)=O)CCN(CC(N)=O)C(Cc2ccc(NC(=S)NCC3CCC(C(=O)NC(Cc4ccc5ccccc5c4)C(=O)NCCCCC(NC(=O)NC(CCC(=O)O)C(=O)O)C(=O)O)CC3)cc2)CN(CC(N)=O)CC1]

or a pharmaceutically acceptable salt thereof; and
(b) a radionuclide bound to the metal chelator, wherein the radionuclide is ²¹²Pb. In some embodiments, the metastatic prostate cancer comprises a metastatic lesion in bone, viscera, lymph node(s), or a combination thereof. In some embodiments, provided herein is a method of treating a metastatic prostate cancer comprising a metastatic lesion in bone, viscera, lymph node(s), or a combination thereof, the method comprising administering ²¹²Pb-Compound 1, or a pharmaceutically acceptable salt thereof, to a subject in need thereof. In some embodiments, the metastatic lesions (e.g., metastatic lesions in bone, viscera, lymph node(s), or a combination thereof) are present in the subject prior to administering ²¹²Pb-Compound 1 or a pharmaceutically acceptable salt thereof, and administering ²¹²Pb-Compound 1 or a pharmaceutically acceptable salt thereof treats, reduces, or eliminates the metastatic lesions. In some embodiments, the metastatic lesion is a bone lesion. In some embodiments, the metastatic lesion is a lesion in the viscera. In some embodiments, the metastatic lesion is a lesion in the lymph node. In some embodiments, the bone lesion is osteolytic bone lesion. In some embodiments, administering ²¹²Pb-Compound 1, or a pharmaceutically acceptable salt thereof, to a subject with prostate cancer prevents or reduces metastasis formation. In some embodiments, administering ²¹²Pb-Compound 1, or a pharmaceutically acceptable salt thereof, to a subject with prostate cancer prevents metastasis formation. In some embodiments, the subject does not have metastatic lesions (e.g., metastatic lesions in bone, viscera, lymph node(s), or a combination thereof) prior to the administering of ²¹²Pb-Compound 1 or a pharmaceutically acceptable salt thereof, and the administering of ²¹²Pb-Compound 1 or a pharmaceutically acceptable salt thereof prevents or reduces the formation of metastatic lesions. In some embodiments, the subject does not have metastatic lesions (e.g., metastatic lesions in bone, viscera, lymph node(s), or a combination thereof) prior to the administering of ²¹²Pb-Compound 1 or a pharmaceutically acceptable salt thereof, and the administering of ²¹²Pb-Compound 1 or a pharmaceutically acceptable salt thereof prevents the formation of metastatic lesions. In some embodiments, provided herein is a method of preventing or reducing prostate cancer metastases in a subject comprising administering ²¹²Pb-Compound 1, or a pharmaceutically acceptable salt thereof, to the subject. In some embodiments, provided herein is a method of preventing prostate cancer metastases in a subject comprising administering ²¹²Pb-Compound 1, or a pharmaceutically acceptable salt thereof, to the subject. In some embodiments, provided herein is a method of treating, reducing, or eliminating prostate cancer metastases in a subject comprising administering ²¹²Pb-Compound 1, or a pharmaceutically acceptable salt thereof, to the subject. In some embodiments, the prostate cancer is metastatic and comprises skeletal metastases prior to administering ²¹²Pb-Compound 1, or a pharmaceutically acceptable salt thereof, to the subject. In some embodiments, the prostate cancer is metastatic and comprises skeletal metastases prior to administering ²¹²Pb-Compound 1, or a pharmaceutically acceptable salt thereof, to the subject, and administering ²¹²Pb-Compound 1, or a pharmaceutically acceptable salt thereof, to the subject prevents or reduces further skeletal metastases. In some embodiments, the prostate cancer is local and the administering of ²¹²Pb-Compound 1, or a pharmaceutically acceptable salt thereof, to the subject prevents or reduces skeletal metastases. In some embodiments, the prostate cancer is metastatic, but does not comprise bone lesions, and the administering of ²¹²Pb-Compound 1, or a pharmaceutically acceptable salt thereof, prevents or reduces further skeletal metastases. In some embodiments, the prostate cancer is metastatic, but does not comprise bone lesions, and the administering of ²¹²Pb-Compound 1, or a pharmaceutically acceptable salt thereof, prevents further skeletal metastases. In some embodiments, the administering of ²¹²Pb-Compound 1 or a salt thereof is more effective in preventing or reducing metastases than a control agent. In some embodiments, the control agent is saline solution. In some embodiments, the control agent is lutetium Lu 177 vipivotide tetraxetan, which is sold under the trade name PLUVICTO®.

In yet another aspect, provided herein is a method of treating a prostate cancer in a subject in need thereof, comprising administering to the subject a radiopharmaceutical conjugate of Formula (I), Formula (I-A), Formula (I-A-1), Formula (I-B-1), or Formula (I-B-2), wherein the radiopharmaceutical conjugate is administered in combination with a second anti-cancer agent. In some embodiments provided herein is a method of treating a prostate cancer in a subject in need thereof, comprising administering to the subject a radiopharmaceutical conjugate, wherein the radiopharmaceutical conjugate comprises,
(a) a compound having the structure:

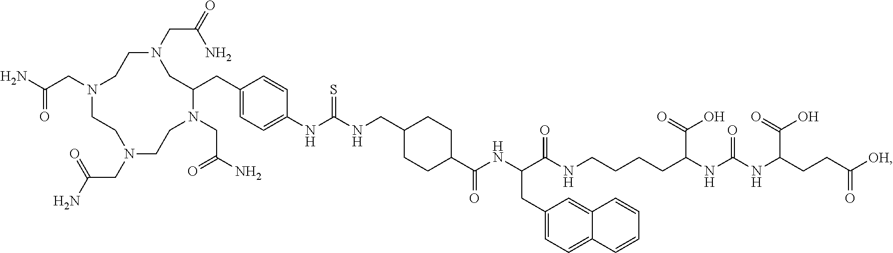 [SMILES: NC(=O)CN1CCN(CC(N)=O)CCN(CC(N)=O)C(Cc2ccc(NC(=S)NCC3CCC(C(=O)NC(Cc4ccc5ccccc5c4)C(=O)NCCCCC(NC(=O)NC(CCC(=O)O)C(=O)O)C(=O)O)CC3)cc2)CN(CC(N)=O)CC1]

or a pharmaceutically acceptable salt thereof; and
(b) a radionuclide bound to the metal chelator, wherein the radionuclide is ²¹²Pb;
   wherein the radiopharmaceutical conjugate is administered in combination with a second anti-cancer agent. In some embodiments, the method comprises administering a therapeutically effective amount of the radiopharmaceutical conjugate to the subject. In some embodiments, the radiopharmaceutical conjugate comprises a compound having the structure of

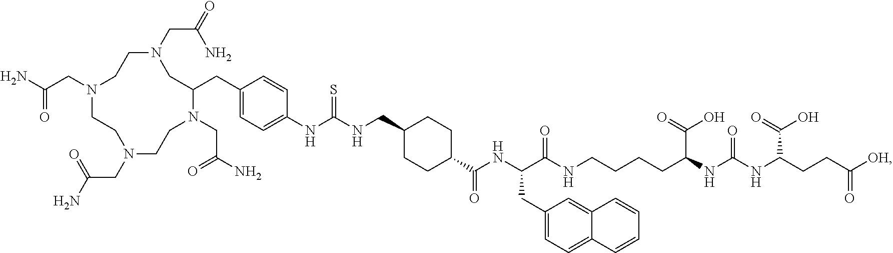

or a pharmaceutically acceptable salt thereof. In some embodiments, the subject has previously received an anti-cancer agent before the administering of the radiopharmaceutical conjugate, and wherein the anti-cancer agent is external beam radiation, chemotherapy, hormonal therapy, or a combination thereof. In some embodiments, the anti-cancer therapy is external beam radiation. In some embodiments, the anti-cancer therapy is chemotherapy. In some embodiments, the chemotherapy is a taxane (for example, docetaxel). In some embodiments, the anti-cancer therapy is hormonal therapy (for example, bicalutamide. flutamide, and goserelin). In some embodiments, the anti-cancer therapy is an androgen receptor axis targeted agent. In some embodiments, the second anti-cancer agent is administered concurrently or sequentially with the radiopharmaceutical conjugate. In some embodiments, the second anti-cancer agent is administered sequentially (e.g., after) the radiopharmaceutical conjugate. In some embodiments, the second anti-cancer agent is administered concurrently with the radiopharmaceutical conjugate. In some embodiments, the second anti-cancer agent is administered prior to administering the radiopharmaceutical conjugate. In some embodiments, the second anti-cancer agent is an androgen receptor-axis targeted agent. In some embodiments, the second anti-cancer agent is abiraterone, enzalutamide, nilutamide, flutamide, bicalutamide, ARN 509, galeterone, orteronel, or a salt thereof. In some embodiments, the second anti-cancer agent is enzalutamide, goserelin, leuprorelin, or a salt thereof. In some embodiments, the radiopharmaceutical conjugate is administered an amount of 50 kBq to 500 MBq per dose. In some embodiments, the radiopharmaceutical conjugate is administered an amount of 100 kBq to 500 MBq per dose. In some embodiments, the radiopharmaceutical conjugate is administered in an amount of about 1 MBq to 500 MBq per dose. In some embodiments, the radiopharmaceutical conjugate is administered in an amount of about 10 MBq to 500 MBq per dose. In some embodiments, the radiopharmaceutical conjugate is administered in an amount of about 50 MBq to 500 MBq per dose. In some embodiments, the radiopharmaceutical conjugate is administered in an amount of about 75 MBq to about 300 MBq per dose. In some embodiments, the radiopharmaceutical conjugate is administered at a dose of about 10 MBq. In some embodiments, the radiopharmaceutical conjugate is administered at a dose of about 100 MBq. In some embodiments, the radiopharmaceutical conjugate is administered without an agent for treating xerostomia. In some embodiments, the agent for treating xerostomia includes agents for treating dry mouth, aptyalism, or dry throat (e.g., spray substitution of saliva). In some embodiments, the subject is not administered the agent for treating xerostomia. In some embodiments, administering the radiopharmaceutical conjugate reduces a risk of xerostomia in the subject, optionally wherein the risk is reduced relative to the administering of ¹⁷⁷Lu-PSMA-617 or ²²⁵Ac-PSMA-617. In some embodiments, the risk is relative to the administering of ²²⁵Ac-PSMA-617. In some embodiments, the subject has received a targeted radiopharmaceutical therapy prior to administering of the radiopharmaceutical conjugate. In some embodiments, the targeted radiopharmaceutical therapy comprises ¹⁷⁷Lu-PSMA-617 or ²²⁵Ac-PSMA-617. In some embodiments, the targeted radiopharmaceutical therapy comprises ¹⁷⁷Lu-PSMA-617. In some embodiments, the subject has progressive prostate cancer after prior treatment with ¹⁷⁷Lu-PSMA-617 or ²²⁵Ac-PSMA-617. In some embodiments, the subject is fasted prior to administering of the radiopharmaceutical conjugate. In some embodiments, the administering of the radiopharmaceutical conjugate does not cause or increase the risk of xerostomia in the subject. In some embodiments, the radiopharmaceutical conjugate is not significantly taken up in a salivary gland in the subject. In some embodiments, the subject does not develop xerostomia after the administering of the radiopharmaceutical conjugate. In some embodiments, the subject was previously diagnosed with xerostomia prior to administering the radiopharmaceutical conjugate. In some embodiments, the subject is susceptible of developing xerostomia. In some embodiments, the administering does not cause any one or more of xerostomia, radiation nephropathy, or bone marrow toxicity, or hematological toxicity in the subject. In some embodiments, the prostate cancer is metastatic castrate resistant prostate cancer (mCRPC). In some embodiments, the prostate cancer is progressive mCRPC. In some embodiments, administering the radiopharmaceutical conjugate prevents or reduces the formation of a metastatic lesion in the subject. In some embodiments, the metastatic lesion comprises a metastatic lesion in bone, viscera, lymph node(s), or a combination thereof. In some embodiments, the metastatic lesion is a metastatic bone lesion. In some embodiments, the metastatic lesion is present in the subject prior to administering the radiopharmaceutical conjugate, and administering the radiopharmaceutical conjugate treats, reduces, or eliminates the metastatic lesion. In some embodiments, the administering prevents or reduces metastasis formation. In some embodiments, the subject does not have a metastatic lesion (e.g., metastatic lesions in bone, viscera, lymph node(s), or a combination thereof) prior to administering the radiopharmaceutical conjugate, and the administering prevents or reduces formation of the metastatic lesions in the subject. In some embodiments, the prostate cancer is metastatic and comprises skeletal metastases prior to administering the radiopharmaceutical conjugate to the subject, and the administering prevents or reduces further skeletal metastases. In some embodiments, the prostate cancer is local and administering the radiopharmaceutical conjugate to the subject prevents or reduces skeletal metastases. In some embodiments, the prostate cancer is metastatic, but does not comprise bone lesions, and administering the radiopharmaceutical conjugate to the subject prevents or reduces further skeletal metastases. In some embodiments, the radiopharmaceutical conjugate is administered via intravenous injection. In some embodiments, the radiopharmaceutical conjugate is administered via bolus intravenous injection.

In another aspect, provided herein is a method of preventing or reducing the formation of a metastatic lesion in a subject in need thereof, comprising administering to the subject a radiopharmaceutical conjugate comprising
(a) a compound of Formula (I):

TL-L-R^{M} Formula (I)
or a pharmaceutically acceptable salt thereof, wherein:
TL is a PSMA targeting ligand comprising a urea;
L is a bivalent linking moiety; and
R^{M} is a metal chelator; and
(b) a radionuclide bound to the metal chelator, wherein the radionuclide is ²¹Pb.
   In some embodiments, the subject has prostate cancer. In some embodiments, the metastatic lesion is a metastatic bone lesion. In some embodiments, the prostate cancer is metastatic and comprises a metastatic lesion in bone, viscera, lymph node(s), or a combination thereof. In some embodiments, the metastatic lesion is present in the subject prior to administering the radiopharmaceutical conjugate, and administering the radiopharmaceutical conjugate treats, reduces, or eliminates the metastatic lesion. In some embodiments, the administering prevents or reduces metastasis formation. In some embodiments, the subject does not have a metastatic lesion (e.g., metastatic lesions in bone, viscera, lymph node(s), or a combination thereof) prior to administering the radiopharmaceutical conjugate, and the administering prevents or reduces formation of the metastatic lesions in the subject. In some embodiments, the prostate cancer is metastatic and comprises skeletal metastases prior to administering the radiopharmaceutical conjugate to the subject, and the administering prevents or reduces further skeletal metastases. In some embodiments, the prostate cancer is local and administering the radiopharmaceutical conjugate to the subject prevents or reduces skeletal metastases. In some embodiments, the prostate cancer is metastatic, but does not comprise bone lesions, and administering the radiopharmaceutical conjugate to the subject prevents or reduces further skeletal metastases.

In addition to the methods of treatment described above, the radiopharmaceutical conjugates described herein (e.g., a radiopharmaceutical conjugate of Formula (I), Formula (I-A), Formula (I-A-1), Formula (I-B-1), or Formula (I-B-2) can be used to image, and/or as part of a treatment for diseases. Conjugates for imaging applications, e.g., single-photon emission computed tomography (SPECT) and positron emission tomography (PET), can comprise any radionuclide suitable for use as imaging isotopes. Accordingly, the conjugate can be administered to confirm target (e.g., PSMA) expression in a subject's tissues.

In one aspect, provided herein are methods for diagnosing or imaging a subject harboring a PSMA expressing cancer or tumor comprising administering to the subject a radiopharmaceutical described herein, or a pharmaceutically acceptable salt or solvate thereof. In one aspect, provided herein are methods for imaging a PSMA expressing cancer or tumor comprising administering to the subject a radiopharmaceutical described herein, or a pharmaceutically acceptable salt or solvate thereof. In some embodiments, the method further comprises selecting or confirming that a tumor in the subject expresses PSMA. In some embodiments, the method further comprises measuring the concentration of the radiopharmaceutical accumulated in the subject. In some embodiments, the method further comprises measuring the amount of radiation emitted from the radionuclide. In some embodiments, the method further comprises analyzing the elimination or clearance profile of the radiopharmaceutical in the subject. In some embodiments, the method further comprises measuring an elimination half-life of the radiopharmaceutical in the patient. In some embodiments, the method further comprises analyzing the clearance profile of the radiopharmaceutical in the subject. For example, radiopharmaceuticals of the present disclosure can be administered for patient selection purposes, such as to confirm the tumor has the appropriate expression of the PSMA target. As another example, radiopharmaceuticals of the present disclosure can be administered to a subject so that the subject's care team can make sure the radiopharmaceutical is cleared from the body in a suitable timeframe so that undesired irradiation of other tissues is minimized.

In one aspect, provided herein is a method of treating a prostate cancer in a subject in need thereof, comprising administering
(a) an imaging radiopharmaceutical agent comprising a first radionuclide; and
(b) a radiopharmaceutical conjugate comprising:
   (i) a compound having the structure:

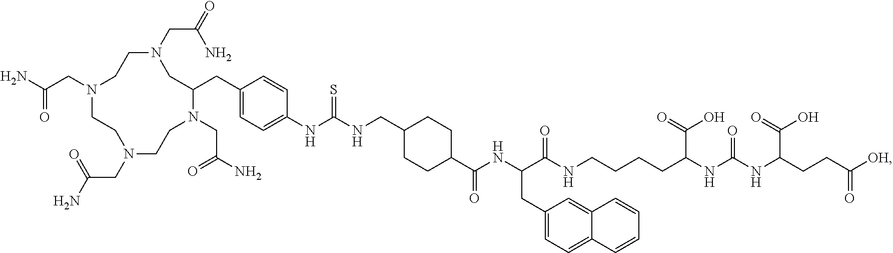 [SMILES: NC(=O)CN1CCN(CC(N)=O)CCN(CC(N)=O)C(Cc2ccc(NC(=S)NCC3CCC(C(=O)NC(Cc4ccc5ccccc5c4)C(=O)NCCCCC(NC(=O)NC(CCC(=O)O)C(=O)O)C(=O)O)CC3)cc2)CN(CC(N)=O)CC1]

or a pharmaceutically acceptable salt thereof; and
(ii) a second radionuclide bound to the metal chelator, wherein the second radionuclide is ²¹²Pb;
   wherein the imaging radiopharmaceutical agent is administered to the patient prior to the administering of the radiopharmaceutical conjugate. In some embodiments, the imaging radiopharmaceutical agent is ⁶⁸Ga-PSMA-11, capromab pendetide, ⁸⁹Zr-J591, ¹¹¹In-J591, IAB2M, ¹⁸F-DCFPyl, ^{99m}Tc-MIP-1404, ¹⁸F-flotufolastat, ¹⁸F-piflufolastat, or ¹⁸F-PSMA-1007, or a pharmaceutically acceptable salt thereof. In some embodiments, the imaging radiopharmaceutical agent is ⁶⁸Ga-PSMA-11. In some embodiments, the imaging radiopharmaceutical agent is ¹⁸F-PSMA-1007. In some embodiments, the imaging radiopharmaceutical agent comprises a compound having the structure of

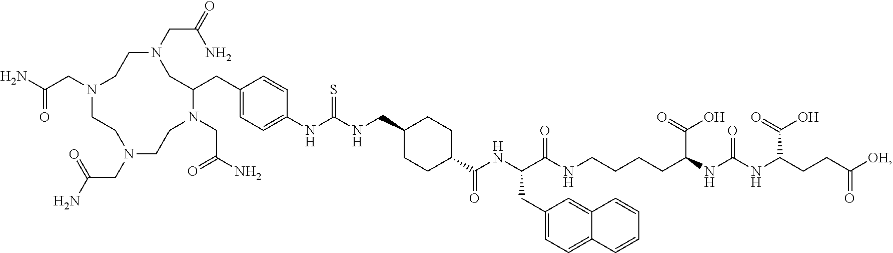

or a pharmaceutically acceptable salt thereof; and wherein the first radionuclide is ²¹²Pb or ²⁰³Pb. In some embodiments, the first radionuclide is complexed with the TCMC. In some embodiments, the first radionuclide is ²¹²Pb. In some embodiments, the first radionuclide is ²⁰³Pb. In some embodiments, the method comprises administering a therapeutically effective amount of the radiopharmaceutical conjugate to the subject. In some embodiments, the radiopharmaceutical conjugate comprises a compound having the structure of

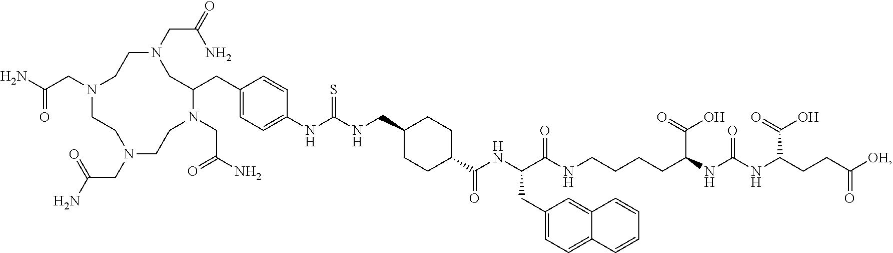

or a pharmaceutically acceptable salt thereof. In some embodiments, the subject has previously received an anti-cancer agent before the administering of the radiopharmaceutical conjugate, and wherein the anti-cancer agent is external beam radiation, chemotherapy, hormonal therapy, or a combination thereof. In some embodiments, the anti-cancer therapy is external beam radiation. In some embodiments, the anti-cancer therapy is chemotherapy. In some embodiments, the chemotherapy is a taxane (for example, docetaxel). In some embodiments, the anti-cancer therapy is hormonal therapy (for example, bicalutamide. flutamide, and goserelin). In some embodiments, the anti-cancer therapy is an androgen receptor axis targeted agent. In some embodiments, the radiopharmaceutical conjugate is administered in combination with a second anti-cancer agent, and wherein the second anti-cancer agent is administered concurrently or sequentially with the radiopharmaceutical conjugate. In some embodiments, the second anti-cancer agent is administered sequentially (e.g., after) the radiopharmaceutical conjugate. In some embodiments, the second anti-cancer agent is administered concurrently with the radiopharmaceutical conjugate. In some embodiments, the second anti-cancer agent is administered prior to administering the radiopharmaceutical conjugate. In some embodiments, the second anti-cancer agent is an androgen receptor-axis targeted agent. In some embodiments, the second anti-cancer agent is abiraterone, enzalutamide, nilutamide, flutamide, bicalutamide, ARN 509, galeterone, orteronel, or a salt thereof. In some embodiments, the second anti-cancer agent is enzalutamide, goserelin, leuprorelin, or a salt thereof. In some embodiments, the radiopharmaceutical conjugate is administered an amount of 50 kBq to 500 MBq per dose. In some embodiments, the radiopharmaceutical conjugate is administered an amount of 100 kBq to 500 MBq per dose. In some embodiments, the radiopharmaceutical conjugate is administered in an amount of about 1 MBq to 500 MBq per dose. In some embodiments, the radiopharmaceutical conjugate is administered in an amount of about 10 MBq to 500 MBq per dose. In some embodiments, the radiopharmaceutical conjugate is administered in an amount of about 50 MBq to 500 MBq per dose. In some embodiments, the radiopharmaceutical conjugate is administered in an amount of about 75 MBq to about 300 MBq per dose. In some embodiments, the radiopharmaceutical conjugate is administered at a dose of about 10 MBq. In some embodiments, the radiopharmaceutical conjugate is administered at a dose of about 100 MBq. In some embodiments, the radiopharmaceutical conjugate is administered without an agent for treating xerostomia. In some embodiments, the agent for treating xerostomia includes agents for treating dry mouth, aptyalism, or dry throat (e.g., spray substitution of saliva). In some embodiments, the subject is not administered the agent for treating xerostomia. In some embodiments, administering the radiopharmaceutical conjugate reduces a risk of xerostomia in the subject, optionally wherein the risk is reduced relative to the administering of ¹⁷⁷Lu-PSMA-617 or ²²⁵Ac-PSMA-617. In some embodiments, the risk is relative to the administering of ²²⁵Ac-PSMA-617. In some embodiments, the subject has received a targeted radiopharmaceutical therapy prior to administering of the radiopharmaceutical conjugate. In some embodiments, the targeted radiopharmaceutical therapy comprises ¹⁷⁷Lu-PSMA-617 or ²²⁵Ac-PSMA-617. In some embodiments, the targeted radiopharmaceutical therapy comprises ¹⁷⁷Lu-PSMA-617. In some embodiments, the subject has progressive prostate cancer after prior treatment with ¹⁷⁷Lu-PSMA-617 or ²²⁵Ac-PSMA-617. In some embodiments, the subject is fasted prior to administering of the radiopharmaceutical conjugate. In some embodiments, the administering of the radiopharmaceutical conjugate does not cause or increase the risk of xerostomia in the subject. In some embodiments, the radiopharmaceutical conjugate is not significantly taken up in a salivary gland in the subject. In some embodiments, the subject does not develop xerostomia after the administering of the radiopharmaceutical conjugate. In some embodiments, the subject was previously diagnosed with xerostomia prior to administering the radiopharmaceutical conjugate. In some embodiments, the subject is susceptible of developing xerostomia. In some embodiments, the administering does not cause any one or more of xerostomia, radiation nephropathy, or bone marrow toxicity, or hematological toxicity in the subject. In some embodiments, the prostate cancer is metastatic castrate resistant prostate cancer (mCRPC). In some embodiments, the prostate cancer is progressive mCRPC. In some embodiments, administering the radiopharmaceutical conjugate prevents or reduces the formation of a metastatic lesion in the subject. In some embodiments, the metastatic lesion comprises a metastatic lesion in bone, viscera, lymph node(s), or a combination thereof. In some embodiments, the metastatic lesion is a metastatic bone lesion. In some embodiments, the metastatic lesion is present in the subject prior to administering the radiopharmaceutical conjugate, and administering the radiopharmaceutical conjugate treats, reduces, or eliminates the metastatic lesion. In some embodiments, the administering prevents or reduces metastasis formation. In some embodiments, the subject does not have a metastatic lesion (e.g., metastatic lesions in bone, viscera, lymph node(s), or a combination thereof) prior to administering the radiopharmaceutical conjugate, and the administering prevents or reduces formation of the metastatic lesions in the subject. In some embodiments, the prostate cancer is metastatic and comprises skeletal metastases prior to administering the radiopharmaceutical conjugate to the subject, and the administering prevents or reduces further skeletal metastases. In some embodiments, the prostate cancer is local and administering the radiopharmaceutical conjugate to the subject prevents or reduces skeletal metastases. In some embodiments, the prostate cancer is metastatic, but does not comprise bone lesions, and administering the radiopharmaceutical conjugate to the subject prevents or reduces further skeletal metastases. In some embodiments, the radiopharmaceutical conjugate is administered via intravenous injection. In some embodiments, the radiopharmaceutical conjugate is administered via bolus intravenous injection.

In some embodiments, the radiopharmaceutical conjugate described herein (e.g., a radiopharmaceutical conjugate of Formula (I), Formula (I-A), Formula (I-A-1), Formula (I-B-1), or Formula (I-B-2) can be administered alone or in combination with one or more additional therapeutic agents. For example, the combination therapy can include a radiopharmaceutical conjugate described herein co-formulated with, and/or co-administered with, one or more additional therapeutic agents, e.g., a second anti-cancer agent. In some embodiments, the second anti-cancer agent is an androgen receptor-axis targeted agent. In some embodiments, the second anti-cancer agent is abiraterone, enzalutamide, nilutamide, flutamide, bicalutamide, ARN 509, galeterone, orteronel, or a salt thereof. In some embodiments, the second anti-cancer agent is enzalutamide, goserelin, leuprorelin, or a salt thereof. Such combination therapies may advantageously utilize lower dosages of the administered therapeutic agents, thus avoiding possible toxicities or complications associated with the various monotherapies.

When administered in combination, two (or more) different treatments can be delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concurrent delivery.” In some embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, provided herein is a method of treating a cancer in a subject in need thereof, wherein the subject has previously received an anti-cancer agent. In some embodiments, the cancer is prostate cancer. In some embodiments, the method comprises administering to the subject a radiopharmaceutical conjugate described herein (e.g., ²¹²Pb-Compound 1). In some embodiments, the subject has previously received an anti-cancer agent selected from external beam radiation, chemotherapy, and hormonal therapy, or a combination thereof. In some embodiments, the previous anti-cancer therapy is external beam radiation. In some embodiments, the previous anti-cancer therapy is chemotherapy. In some embodiments, the previous chemotherapy is a taxane (for example, docetaxel). In some embodiments, the previous anti-cancer therapy is hormonal therapy (for example, bicalutamide. flutamide, and goserelin). In some embodiments, the anti-cancer therapy is an androgen receptor axis targeted agent.

In some embodiments, the subject has a metastatic lesion visualized by PSMA PET imaging. In some embodiments, the PSMA-PET imaging co-localizes with ²¹²Pb-Compound 1 imaging.

In some embodiments, the subject has prostate cancer. In some embodiments, the subject has metastatic prostate cancer. In some embodiments, the subject has metastatic castrate resistant prostate cancer. In some embodiments, the cancer comprises PSMA-avid lesions demonstrated by PSMA PET/CT. In some embodiments, the cancer is stage IV metastatic castrate resistant prostate cancer. In some embodiments, the prostate cancer is locally extensive. In some embodiments, the prostate cancer comprises bone metastases. In some embodiments, the tumor is classified via the TNM classification system as T3NoM1b or T3N×M1b.

In some embodiments, the subject has an ECOG performance status of 0-2. In some embodiments, the subject has an ECOG performance status of 1. In some embodiments, the subject has a TEAE after administration of ²¹²Pb-Compound 1. In some embodiments, the TEAE is grade 1 or grade 2. In some embodiments, the TEAE are unrelated to ²¹²Pb-Compound 1 administration. The TEAE is selected from an injury, poisoning, or procedural complication (e.g., tooth fracture), a gastrointestinal disorder (e.g., abdominal pain, constipation, vomiting, etc.), a generalized disorder and administration site condition (e.g., localized oedema, etc.), a skin and subcutaneous tissue disorder (e.g., erythema, etc.), or a combination thereof. In some embodiments, the subject is administered a therapeutic agent for the treatment of the injury, poisoning, or procedural complication, the gastrointestinal disorder, the generalized disorder and administration site condition, or the skin and subcutaneous tissue disorder. In some embodiments, the TEAE does not include xerostomia. In some embodiments, the TEAE does not include xerostomia of any grade severity. In some embodiments, the TEAE does not include grade 1, 2, 3, or 4 xerostomia. In some embodiments, the TEAE does not include grade 2, 3, or 4 xerostomia. In some embodiments, the TEAE does not include grade 3 or 4 xerostomia. In some embodiments, the subject does not experience an adverse reaction, a serious adverse event, a suspected unexpected serious adverse event, or a death caused by administering ²¹²Pb-Compound 1. In some embodiments, the subject has a life expectancy of greater than 6 months. In some embodiments, the subject has adequate hematopoietic, kidney, and liver function.

In some embodiments, the subject has a prior orchiectomy. In some embodiments, the subject has a serum testosterone level of <50 ng/DL or <1.7 nmol/L. In some embodiments, the subject has previously received 1 or 2 regimens of taxane therapy before the administering of the radiopharmaceutical conjugate. In some embodiments, the subject is ineligible for taxane therapy. In some embodiments, the subject has at least one PSMA-avid distant metastatic lesion. In some embodiments, the subject has previously been treated with at least one androgen receptor axis targeted agent prior to the administering of the radiopharmaceutical conjugate. In some embodiments, the androgen receptor axis targeted agent is a novel androgen receptor axis targeted agent.

In some embodiments, the subject does not have a urinary obstruction. In some embodiments, the subject does not have an untreated CNS metastases. In some embodiments, the subject does not have a symptomatic medullary cord compression. In some embodiments, the subject does not have a diffuse bone or bone marrow involvement. In some embodiments, the subject does not have a radiation hypersensitivity. In some embodiments, the subject does not have a history of myelodysplastic syndrome. In some embodiments, the subject does not have a treatment-related acute myeloid leukemia. In some embodiments, the subject does not have a non-prostate cancer requiring treatment within 2 years prior to administering the radiopharmaceutical conjugate (excepting for treated basal cell carcinoma, squamous cell carcinoma, or carcinoma in situ). In some embodiments, the subject does not have an infection of CTCAE v5.0 Grade 2 not responding to therapy. In some embodiments, the subject does not have an infection of CTCAE Grade >2. In some embodiments, the subject does not have a human immunodeficiency virus. In some embodiments, the subject does not have active hepatitis B virus. In some embodiments, the subject does not have active hepatitis C virus. In some embodiments, the subject does not have a non-healing wound or bone fracture. In some embodiments, the subject does not have a major surgery within 4 weeks prior to administering the radiopharmaceutical conjugate. In some embodiments, the subject does not have impaired cardiac function or clinically significant cardiac disease. In some embodiments, the subject has not received prior systemic anti-cancer therapy or investigational therapy within 4 weeks prior to administering the radiopharmaceutical conjugate, with the exception of luteinizing hormone-releasing hormone or gonadotropin-releasing hormone. In some embodiments, the subject has not received prior PSMA-targeted radiopharmaceutical therapy, with the exception of ¹⁷⁷Lu-PSMA-617 or ²²⁵Ac-PSMA-617. In some embodiments, the subject has not received prior treatment with systemic radiopharmaceutical therapy within 6 months prior to administering the radiopharmaceutical conjugate. In some embodiments, the subject has not received prior definitive radiotherapy within 6 weeks prior to administering the radiopharmaceutical conjugate. In some embodiments, the subject has not received external beam radiotherapy to a critical organ which has exceeded the organ tolerance dose limit. In some embodiments, the subject has not received hemi-body irradiation within 6 months prior to administering the radiopharmaceutical conjugate. In some embodiments, the subject has not received high-dose chemotherapy requiring hematopoietic-stem-cell-rescue. In some embodiments, the subject has not received an autologous or allogenic stem-cell transplant. In some embodiments, the subject does not have ongoing toxicity CTCAE Grade 2 or higher due to prior anti-cancer therapy that is not stabilized. In some embodiments, the subject has not been administered a live vaccine within 4 weeks prior to administration of the radiopharmaceutical conjugate. In some embodiments, the subject has not been administered a biological response modifier, including granulocyte colony-stimulating factor, granulocyte macrophage colony-stimulating factor, and erythropoietin within 3 weeks prior to administering the radiopharmaceutical conjugate. In some embodiments, the subject has not received systemic corticosteroids (e.g., >10 mg of prednisone/prednisolone per day) or herbal products that decrease PSA levels (e.g., saw palmetto) within 4 weeks prior to administering the radiopharmaceutical conjugate.

In some embodiments, ²¹²Pb-Compound 1 is administered to the subject via intravenous injection. In some embodiments, ²¹²Pb-Compound 1 is administered via slow bolus injection. In some embodiments, ²¹²Pb-Compound 1 is administered via slow bolus intravenous injection via a 3-way adapter coupled to a conventional isotonic saline solution.

In some embodiments, the subject is imaged by gamma camera imaging. In some embodiments, the subject is imaged by gamma planar imaging. In some embodiments, the subject is imaged by SPECT/CT imaging.

In some embodiments, in vivo half-life of ²¹²Pb-Compound 1 is determined via blood sampling and whole-body probe measurements. In some embodiments, in clearance of ²¹²Pb-Compound 1 is determined via blood sampling and whole-body probe measurements. In some embodiments, blood sampling and whole-body probe measurements are taken 0-30 minutes, 1-2 hours, 4-6 hours, and 16-24 hours after administration of ²¹²Pb-Compound 1.

In some embodiments, a subject is imaged via ¹⁸F-PSMA-PET/CT prior to ²¹²Pb-Compound 1 administration. In some embodiments, the subject is imaged via ¹⁸F-PSMA-1007. In some embodiments, the ¹⁸F-PSMA-1007 is administered at a dose of 2.5 MBq/kg. In some embodiments, the ¹⁸F-PSMA-1007 is administered at a maximum dose of 300 MBq. In some embodiments, the ¹⁸F-PSMA-1007 is administered at a minimum dose of 200 MBq. In some embodiments, the ¹⁸F-PSMA-1007 is administered at a dose between 200 MBq and 300 MBq.

In some embodiments, a subject has been previously treated with a systemic anti-cancer therapy prior to ²¹²Pb-Compound 1 administration. In some embodiments, the systemic anti-cancer therapy is hormonal therapy or chemotherapy. In some embodiments, the hormonal therapy is bicalutamide (e.g., 150 mg once daily), exulexin (e.g., 250 mg three times a day), zoladex (e.g., 10 mg every 12 weeks). In some embodiments, the chemotherapy is docetaxel (e.g., 150 mg every third week) or docetaxel-21 (e.g., 170 mg).

In some embodiments, a subject administered ²¹²Pb-Compound 1 has a concomitant disease. In some embodiments, the subject has a cardiac disorder (e.g., atrial fibrillation, myocardial ischemia, etc.), a gastrointestinal disorder (e.g., hiatus hernia, etc.), an injury (e.g., spinal compression fracture, etc.), a metabolic or nutrition disorder (e.g., folate deficiency, hypercholesterolemia, etc.), a musculoskeletal or connective tissue disorder (e.g., bone pain, osteoporosis, spinal stenosis, etc.), a nervous system disorder (e.g., polyneuropathy, etc.), a renal or urinary disorder (e.g., lower urinary tract symptoms, etc.), a prior surgical procedure (e.g., artificial urinary sphincter implant, etc.), or a vascular disorder (e.g., hypertension).

In some embodiments, a subject administered ²¹²Pb-Compound 1 is receiving a concomitant medication. In some embodiments, a subject is administered ²¹²Pb-Compound 1 in combination with a concomitant medication. In some embodiments, the concomitant medication is not a chemotherapy, a biologic therapy, an immune-oncology agent, or a radiation therapy. In some embodiments, the concomitant therapy comprises a drug for acid related disorders (e.g., esomeprazole, etc.), a drug for constipation (e.g., macrogol, sodium picosulfate, etc.), a vitamin (e.g., vitamin D, etc.), a mineral supplement (e.g., calcium, including combinations with vitamin D and/or other drugs, etc.), an antithrombotic agent (e.g., acetylsalicylic acid, warfarin, etc.), an antianemic preparation (e.g., cyanocobalamin, folic acid, etc.), an agent acting on the renin-angiotensin system (e.g., candeartan, losartan, diuretics, etc.), a lipid modifying agent (e.g., atorvastin, ezetimibe, etc.), an endocrine therapy (e.g., enzalutamide, goserelin, leuprorelin, etc.), drugs for the treatment of bone diseases (e.g., denosumab, zoledronic acid, etc.), analgesics (e.g., morphine, paracetamol, etc.), or an antihistamine (e.g., alimemazine, etc.).

In some embodiments, ²¹²Pb-Compound 1 is not taken up in the salivary glands of a subject as determined by gamma imaging. In some embodiments, ²¹²Pb-Compound 1 is taken up by the kidneys in the subject as determined by gamma imaging. In some embodiments, the uptake of ²¹²Pb-Compound 1 in the kidneys of a subject is greater than or equal to the uptake of ²¹²Pb-Compound 1 in the liver of the subject. In some embodiments, ²¹²Pb-Compound 1 is not taken up by the spleen of the subject as determined by gamma imaging. In some embodiments, ²¹²Pb-Compound 1 is not taken up by the small intestines of the subject as determined by gamma imaging. In some embodiments, ²¹²Pb-Compound 1 is not taken up by the blood pool of the subject as determined by gamma imaging. In some embodiments, ²¹²Pb-Compound 1 is not taken up by the bladder of the subject as determined by gamma imaging. In some embodiments, ²¹²Pb-Compound 1 is taken up by the bladder of the subject as determined by gamma imaging. In some embodiments, the uptake of ²¹²Pb-Compound 1 in the bladder of a subject is equal to the uptake of ²¹²Pb-Compound 1 in the liver of the subject. In some embodiments, ²¹²Pb-Compound 1 is not taken up by the bone marrow of the subject as determined by gamma imaging. In some embodiments, uptake determined by gamma imaging is determined by visual assessment of a gamma planar imaging or SPECT/CT scan.

### Dose and Frequency of Administration

In one aspect, disclosed herein is a method of treating cancer in a subject in need thereof, wherein the method comprises administering to the subject a radiopharmaceutical conjugate, wherein the radiopharmaceutical conjugate comprises (a) a metal chelator; (b) a target-binding moiety, and (c) a radionuclide bound to the metal chelator, wherein the radionuclide is ²¹²Pb. In some embodiments, the radiopharmaceutical conjugate has the structure of Formula (I), Formula (I-A), Formula (I-A-1), Formula (I-B-1), or Formula (I-B-2). In some embodiments, the radiopharmaceutical conjugate is administered to the subject about every 1 to 7 weeks. In some embodiments, the radiopharmaceutical conjugate is administered to the subject about every 2 to 9 weeks. In some embodiments, the target-binding moiety binds to a PSMA. In some embodiments, the metal chelator is TCMC. In some embodiments, the metal chelator is DOTA. In some embodiments, the radiopharmaceutical conjugate is ²¹²Pb-Compound 1. In some embodiments, the radiopharmaceutical conjugate has a structure of

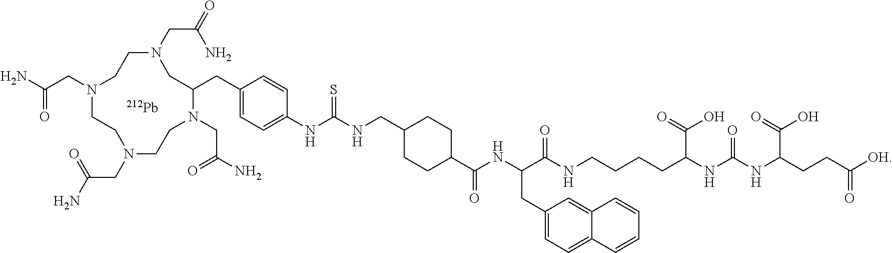

In some embodiments, the radiopharmaceutical conjugate has a structure of

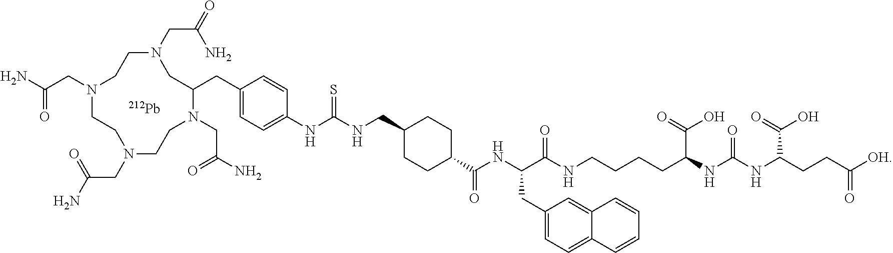

In some embodiments, the radiopharmaceutical conjugate is administered to the subject about once every week, about once every 2 weeks, about once every 3 weeks, about once every 4 weeks, about once every 5 weeks, or about once every 6 weeks. In some embodiments, the radiopharmaceutical conjugate is administered to the subject once a week. In some embodiments, the radiopharmaceutical conjugate is administered to the subject once every 2 weeks. In some embodiments, the radiopharmaceutical conjugate is administered to the subject once every 3 weeks. In some embodiments, the radiopharmaceutical conjugate is administered to the subject once every 4 weeks. In some embodiments, the radiopharmaceutical conjugate is administered to the subject once every 5 weeks. In some embodiments, the radiopharmaceutical conjugate is administered to the subject once every 6 weeks. In some embodiments, the radiopharmaceutical conjugate is administered to the subject once about every week. In some embodiments, the radiopharmaceutical conjugate is administered to the subject once about every 2 weeks. In some embodiments, the radiopharmaceutical conjugate is administered to the subject once about every 3 weeks. In some embodiments, the radiopharmaceutical conjugate is administered to the subject once about every 4 weeks. In some embodiments, the radiopharmaceutical conjugate is administered to the subject once about every 5 weeks. In some embodiments, the radiopharmaceutical conjugate is administered to the subject once about every 6 weeks. In some embodiments, the radiopharmaceutical conjugate is administered to the subject once about every 7 weeks. In some embodiments, the radiopharmaceutical conjugate is administered to the subject at a 2-week interval. In some embodiments, the radiopharmaceutical conjugate is administered to the subject at a 3-week interval. In some embodiments, the radiopharmaceutical conjugate is administered to the subject at a 4-week interval. In some embodiments, the radiopharmaceutical conjugate is administered to the subject at a 5-week interval. In some embodiments, the radiopharmaceutical conjugate is administered to the subject at a 6-week interval. In some embodiments, the radiopharmaceutical conjugate is administered to the subject at a 7-week interval. In some embodiments, the radiopharmaceutical conjugate is administered 2 times (i.e., for 2 cycles), 3 times, (i.e., for 3 cycles), 4 times, (i.e., 4 cycles), 5 times (i.e., 5 cycles), 6 times (i.e., 6 cycles), 7 times (i.e., 7 cycles), 8 times (i.e., 8 cycles), 9 times (i.e., 9 cycles), 10 times (i.e., 10 cycles), 11 times (i.e., 11 cycles), or 12 times (i.e., 12 cycles). In some embodiments, the radiopharmaceutical conjugate is administered 2 times (i.e., for 2 cycles), 3 times, (i.e., for 3 cycles), 4 times, (i.e., 4 cycles), 5 times (i.e., 5 cycles), 6 times (i.e., 6 cycles), 7 times (i.e., 7 cycles), or 8 times (i.e., 8 cycles). In some embodiments, the radiopharmaceutical conjugate is administered for at least one cycle. In some embodiments, the radiopharmaceutical conjugate is administered for 2 cycles. In some embodiments, the radiopharmaceutical conjugate is administered for 2 or more cycles. In some embodiments, the radiopharmaceutical conjugate is administered for 3 cycles. In some embodiments, the radiopharmaceutical conjugate is administered for 4 cycles. In some embodiments, the radiopharmaceutical conjugate is administered for 5 cycles. In some embodiments, the radiopharmaceutical conjugate is administered for 6 cycles. In some embodiments, the radiopharmaceutical conjugate is administered for 7 cycles. In some embodiments, the radiopharmaceutical conjugate is administered for 8 cycles. In some embodiments, the radiopharmaceutical conjugate is administered for 9 cycles. In some embodiments, the radiopharmaceutical conjugate is administered for 10 cycles. In some embodiments, the radiopharmaceutical conjugate is administered for 11 cycles. In some embodiments, the radiopharmaceutical conjugate is administered for 12 cycles. In some embodiments, the radiopharmaceutical conjugate is administered for 13 to 20 cycles.

In one aspect, provided herein is a method of treating a prostate cancer in a subject in need thereof, comprising administering to the subject a radiopharmaceutical conjugate, In some embodiments, the radiopharmaceutical conjugate has the structure of Formula (I), Formula (I-A), Formula (I-A-1), Formula (I-B-1), or Formula (I-B-2), wherein the radiopharmaceutical conjugate is administered to the subject in an amount between 50 kBq to 500 MBq per dose. In some embodiments, provided herein is a method of treating a prostate cancer in a subject in need thereof, comprising administering to the subject a radiopharmaceutical conjugate, wherein the radiopharmaceutical conjugate comprises,
(a) a compound having the structure:

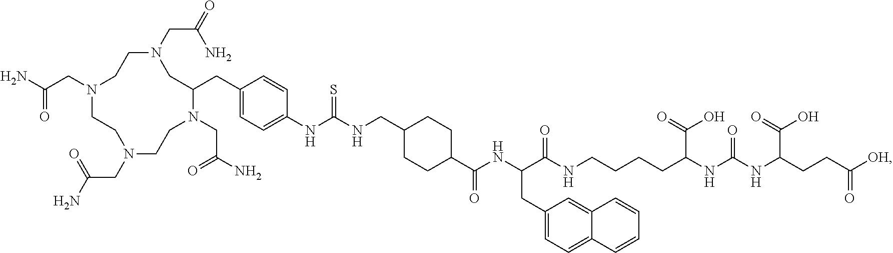 [SMILES: NC(=O)CN1CCN(CC(N)=O)CCN(CC(N)=O)C(Cc2ccc(NC(=S)NCC3CCC(C(=O)NC(Cc4ccc5ccccc5c4)C(=O)NCCCCC(NC(=O)NC(CCC(=O)O)C(=O)O)C(=O)O)CC3)cc2)CN(CC(N)=O)CC1]

or a pharmaceutically acceptable salt thereof; and
(b) a radionuclide bound to the metal chelator, wherein the radionuclide is ²¹²Pb;
   wherein the radiopharmaceutical conjugate is administered to the subject in an amount between 50 kBq to 500 MBq per dose. In some embodiments, the radiopharmaceutical conjugate has a structure of

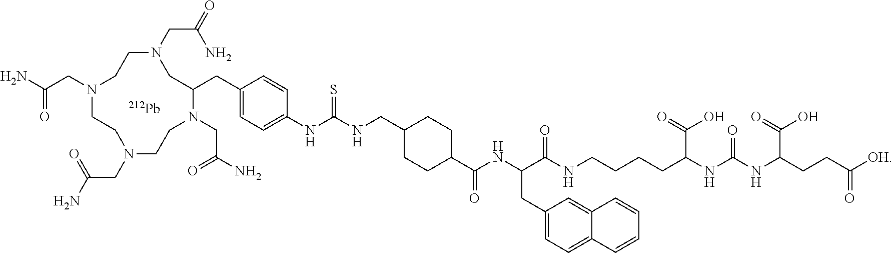

In some embodiments, the radiopharmaceutical conjugate has a structure of

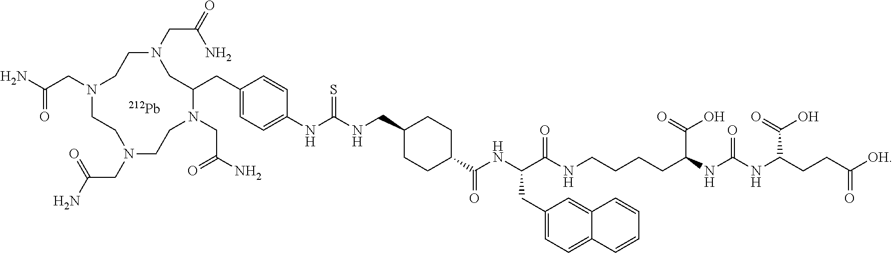

In some embodiments, the radiopharmaceutical conjugate is administered in an amount of about 100 kBq to 500 MBq per dose. In some embodiments, the radiopharmaceutical conjugate is administered in an amount of about 1 MBq to 500 MBq per dose. In some embodiments, the radiopharmaceutical conjugate is administered in an amount of about 10 MBq to 500 MBq per dose. In some embodiments, the radiopharmaceutical conjugate is administered in an amount of about 50 MBq to 500 MBq per dose. In some embodiments, the radiopharmaceutical conjugate is administered in an amount of about 10 MBq per dose. In some embodiments, the radiopharmaceutical conjugate is administered in an amount of about 100 MBq per dose. In some embodiments, the radiopharmaceutical conjugate is administered in an amount of about 10 MBq to about 75 MBq. In some embodiments, the radiopharmaceutical conjugate is administered in an amount of about 25 MBq to about 100 MBq. In some embodiments, the radiopharmaceutical conjugate is administered in an amount of about 50 MBq to about 300 MBq. In some embodiments, the radiopharmaceutical conjugate is administered in an amount of about 50 MBq to about 250 MBq. In some embodiments, the radiopharmaceutical conjugate is administered in an amount of about 50 MBq to about 200 MBq. In some embodiments, the radiopharmaceutical conjugate is administered in an amount of about 50 MBq to about 150 MBq. In some embodiments, the radiopharmaceutical conjugate is administered in an amount of about 75 MBq to about 175 MBq. In some embodiments, the radiopharmaceutical conjugate is administered in an amount of about 100 MBq to about 200 MBq. In some embodiments, the radiopharmaceutical conjugate is administered in an amount of about 125 MBq to about 225 MBq. In some embodiments, the radiopharmaceutical conjugate is administered in an amount of about 150 MBq to about 250 MBq. In some embodiments, the radiopharmaceutical conjugate is administered in an amount of about 175 MBq to about 275 MBq. In some embodiments, the radiopharmaceutical conjugate is administered in an amount of about 200 MBq to about 300 MBq. In some embodiments, the radiopharmaceutical conjugate is administered in an amount of about 225 MBq to about 325 MBq. In some embodiments, the radiopharmaceutical conjugate is administered in an amount of about 250 MBq to about 350 MBq. In some embodiments, the radiopharmaceutical conjugate is administered in an amount of about 275 MBq to about 375 MBq. In some embodiments, the radiopharmaceutical conjugate is administered in an amount of about 300 MBq to about 400 MBq. In some embodiments, the radiopharmaceutical conjugate is administered in an amount of about 25 MBq to about 75 MBq. In some embodiments, the radiopharmaceutical conjugate is administered in an amount of about 50 MBq to about 100 MBq. In some embodiments, the radiopharmaceutical conjugate is administered in an amount of about 75 MBq to about 125 MBq. In some embodiments, the radiopharmaceutical conjugate is administered in an amount of about 100 MBq to about 150 MBq. In some embodiments, the radiopharmaceutical conjugate is administered in an amount of about 125 MBq to about 175 MBq. In some embodiments, the radiopharmaceutical conjugate is administered in an amount of about 150 MBq to about 200 MBq. In some embodiments, the radiopharmaceutical conjugate is administered in an amount of about 175 MBq to about 225 MBq. In some embodiments, the radiopharmaceutical conjugate is administered in an amount of about 200 MBq to about 250 MBq. In some embodiments, the radiopharmaceutical conjugate is administered in an amount of about 225 MBq to about 275 MBq. In some embodiments, the radiopharmaceutical conjugate is administered in an amount of about 250 MBq to about 300 MBq. In some embodiments, the radiopharmaceutical conjugate is administered in an amount of about 275 MBq to about 325 MBq. In some embodiments, the radiopharmaceutical conjugate is administered in an amount of about 300 MBq to about 350 MBq. In some embodiments, the radiopharmaceutical conjugate is administered in an amount of about 325 MBq to about 375 MBq. In some embodiments, the radiopharmaceutical conjugate is administered in an amount of about 350 MBq to about 400 MBq. In some embodiments, the radiopharmaceutical conjugate is administered in an amount of about 75 MBq to about 300 MBq per dose. In some embodiments, the radiopharmaceutical conjugate is administered in an amount of about 75 MBq to about 250 MBq per dose. In some embodiments, the radiopharmaceutical conjugate is administered in an amount of about 75 MBq to about 200 MBq per dose. In some embodiments, the radiopharmaceutical conjugate is administered in an amount of about 100 MBq to about 200 MBq per dose. In some embodiments, the radiopharmaceutical conjugate is administered in an amount of about 70 MBq per dose about 75 MBq per dose, about 80 MBq per dose, about 85 MBq per dose, about 90 MBq per dose, about 95 MBq per dose, about 100 MBq per dose, about 105 MBq per dose, about 110 MBq per dose, about 115 MBq per dose, about 120 MBq per dose, about 125 MBq per dose, about 130 MBq per dose, about 140 MBq per dose, about 145 MBq per dose, about 150 MBq per dose, about 155 MBq per dose, about 160 MBq per dose, about 165 MBq per dose, about 170 MBq per dose, about 175 MBq per dose, about 180 MBq per dose, about 185 MBq per dose, about 190 MBq per dose, about 195 MBq per dose, about 200 MBq per dose, about 205 MBq per dose, about 210 MBq per dose, about 215 MBq per dose, about 220 MBq per dose, about 225 MBq per dose, about 230 MBq per dose, about 235 MBq per dose, about 240 MBq per dose, about 245 MBq per dose, about 250 MBq per dose, about 255 MBq per dose, about 260 MBq per dose, about 265 MBq per dose, about 270 MBq per dose, about 275 MBq per dose, about 280 MBq per dose, about 285 MBq per dose, about 290 MBq per dose, about 295 MBq per dose, or about 300 MBq per dose. In some embodiments, the radiopharmaceutical conjugate is administered in an amount of about 75 MBq per dose, about 100 MBq per dose, about 125 MBq per dose, about 150 MBq per dose, about 175 MBq per dose, about 200 MBq per dose, about 225 MBq per dose, about 250 MBq per dose, about 275 MBq per dose, or about 300 MBq per dose. In some embodiments, the radiopharmaceutical conjugate is administered in an amount of about 75 MBq per dose, about 100 MBq per dose, about 150 MBq per dose, about 200 MBq per dose, about 250 MBq per dose, or about 300 MBq per dose. In some embodiments, the radiopharmaceutical conjugate is administered in an amount of about 75 MBq per dose. In some embodiments, the radiopharmaceutical conjugate is administered in an amount of about 100 MBq per dose. In some embodiments, the radiopharmaceutical conjugate is administered in an amount of about 125 MBq per dose. In some embodiments, the radiopharmaceutical conjugate is administered in an amount of about 150 MBq per dose. In some embodiments, the radiopharmaceutical conjugate is administered in an amount of about 175 MBq per dose. In some embodiments, the radiopharmaceutical conjugate is administered in an amount of about 200 MBq per dose. In some embodiments, the radiopharmaceutical conjugate is administered in an amount of about 225 MBq per dose. In some embodiments, the radiopharmaceutical conjugate is administered in an amount of about 250 MBq per dose. In some embodiments, the radiopharmaceutical conjugate is administered in an amount of about 275 MBq per dose. In some embodiments, the radiopharmaceutical conjugate is administered in an amount of about 300 MBq per dose. In some embodiments, a radiopharmaceutical conjugate described herein is administered to a subject in need thereof as a weight-based dose. In some embodiments, the radiopharmaceutical conjugate is administered in an amount of about 0.02 kBq/kg to about 225 kBq/kg. In some embodiments, a radiopharmaceutical conjugate described herein is administered to a subject in need thereof as a weight-based dose. In some embodiments, the radiopharmaceutical conjugate is administered in an amount of about 0.25 kBq/kg to about 22.5 kBq/kg. In some embodiments, a radiopharmaceutical conjugate described herein is administered to a subject in need thereof as a weight-based dose. In some embodiments, the radiopharmaceutical conjugate is administered in an amount of about 0.25 kBq/kg to about 10 kBq/kg. In some embodiments, the radiopharmaceutical conjugate is administered in an amount of about 0.25 kBq/kg to about 5 kBq/kg. In some embodiments, the radiopharmaceutical conjugate is administered in an amount of about In some embodiments, the radiopharmaceutical conjugate is administered in an amount of about 0.5 kBq/kg to about 5 kBq/kg. In some embodiments, the radiopharmaceutical conjugate is administered in an amount of about In some embodiments, the radiopharmaceutical conjugate is administered in an amount of about 0.75 kBq/kg to about 5 kBq/kg. In some embodiments, the radiopharmaceutical conjugate is administered in an amount of about 1 kBq/kg to about 5 kBq/kg. In some embodiments, the radiopharmaceutical conjugate is administered in an amount of about 1 kBq/kg to about 4.5 kBq/kg. In some embodiments, the radiopharmaceutical conjugate is administered in an amount of about 1 kBq/kg to about 2 kBq/kg, or about 1.5 kBq/kg to about 2.5 kBq/kg, or about 2 kBq/kg to about 3 kBq/kg, or about 2.5 kBq/kg to about 3.5 kBq/kg, or about 3 kBq/kg to about 4 kBq/kg, or about 3.5 kBq/kg to about 4.5 kBq/kg, or about 4 kBq/kg to about 5 kBq/kg. In some embodiments, the radiopharmaceutical conjugate is administered in an amount of about 0.5 kBq/kg to about 1 kBq/kg. In some embodiments, the radiopharmaceutical conjugate is administered in an amount of about 0.75 kBq/kg to about 1.25 kBq/kg. In some embodiments, the radiopharmaceutical conjugate is administered in an amount of about 1 kBq/kg to about 1.5 kBq/kg. In some embodiments, the radiopharmaceutical conjugate is administered in an amount of about 1.25 kBq/kg to about 1.75 kBq/kg. In some embodiments, the radiopharmaceutical conjugate is administered in an amount of about 1.5 kBq/kg to about 2 kBq/kg. In some embodiments, the radiopharmaceutical conjugate is administered in an amount of about 1.75 kBq/kg to about 2.25 kBq/kg. In some embodiments, the radiopharmaceutical conjugate is administered in an amount of about 2 kBq/kg to about 2.5 kBq/kg. In some embodiments, the radiopharmaceutical conjugate is administered in an amount of about 2.25 kBq/kg to about 2.75 kBq/kg. In some embodiments, the radiopharmaceutical conjugate is administered in an amount of about 2.5 kBq/kg to about 3 kBq/kg. In some embodiments, the radiopharmaceutical conjugate is administered in an amount of about 2.75 kBq/kg to about 3.25 kBq/kg. In some embodiments, the radiopharmaceutical conjugate is administered in an amount of about 3 kBq/kg to about 3.5 kBq/kg. In some embodiments, the radiopharmaceutical conjugate is administered in an amount of about 3.25 kBq/kg to about 3.75 kBq/kg. In some embodiments, the radiopharmaceutical conjugate is administered in an amount of about 3.5 kBq/kg to about 4 kBq/kg. In some embodiments, the radiopharmaceutical conjugate is administered in an amount of about 3.75 kBq/kg to about 4.25 kBq/kg. In some embodiments, the radiopharmaceutical conjugate is administered in an amount of about 4 kBq/kg to about 4.5 kBq/kg. In some embodiments, the radiopharmaceutical conjugate is administered in an amount of about 4.25 kBq/kg to about 4.75 kBq/kg. In some embodiments, the radiopharmaceutical conjugate is administered in an amount of about 4.5 kBq/kg to about 5 kBq/kg. In some embodiments, the radiopharmaceutical conjugate is administered in an amount of about 0.25 kBq/kg. In some embodiments, the radiopharmaceutical conjugate is administered in an amount of about 0.5 kBq/kg. In some embodiments, the radiopharmaceutical conjugate is administered in an amount of about 0.75 kBq/kg. In some embodiments, the radiopharmaceutical conjugate is administered in an amount of about 1 kBq/kg. In some embodiments, the radiopharmaceutical conjugate is administered in an amount of about 1.25 kBq/kg. In some embodiments, the radiopharmaceutical conjugate is administered in an amount of about 1.5 kBq/kg. In some embodiments, the radiopharmaceutical conjugate is administered in an amount of about 1.75 kBq/kg. In some embodiments, the radiopharmaceutical conjugate is administered in an amount of about 2 kBq/kg. In some embodiments, the radiopharmaceutical conjugate is administered in an amount of about 2.25 kBq/kg. In some embodiments, the radiopharmaceutical conjugate is administered in an amount of about 2.5 kBq/kg. In some embodiments, the radiopharmaceutical conjugate is administered in an amount of about 2.75 kBq/kg. In some embodiments, the radiopharmaceutical conjugate is administered in an amount of about 3 kBq/kg. In some embodiments, the radiopharmaceutical conjugate is administered in an amount of about 3.25 kBq/kg. In some embodiments, the radiopharmaceutical conjugate is administered in an amount of about 3.5 kBq/kg. In some embodiments, the radiopharmaceutical conjugate is administered in an amount of about 3.75 kBq/kg. In some embodiments, the radiopharmaceutical conjugate is administered in an amount of about 4 kBq/kg. In some embodiments, the radiopharmaceutical conjugate is administered in an amount of about 4.25 kBq/kg. In some embodiments, the radiopharmaceutical conjugate is administered in an amount of about 4.5 kBq/kg. In some embodiments, the radiopharmaceutical conjugate is administered in an amount of about 4.75 kBq/kg. In some embodiments, the radiopharmaceutical conjugate is administered in an amount of about 5 kBq/kg.

In some embodiments, the subject is administered folate in combination with a radiopharmaceutical described herein, In some embodiments, the subject is administered folate prior to, concurrently with, or after administering a radiopharmaceutical conjugate described herein. In some embodiments, the folate is administered concurrently (i.e., at the same time) as the radiopharmaceutical conjugate. In some embodiments, the subject is administered folate after administering a radiopharmaceutical conjugate described herein. In some embodiments, the folate is administered 0.25, 0.5, 1, 1.5, 2, 2.5, 3, 4, 5, 6, 7, 8, 9, 10, 11, or 12 hours after administering the radiopharmaceutical conjugate. In some embodiments, the radiopharmaceutical conjugate has the structure of Formula (I), Formula (I-A), Formula (I-A-1), Formula (I-B-1), or Formula (I-B-2). In some embodiments, the folate is administered up to 2 days prior to administering the radiopharmaceutical conjugate. In some embodiments, the folate is administered up to 24 hours prior to administering the radiopharmaceutical conjugate. In some embodiments, the folate is administered up to 12 hours prior to administering the radiopharmaceutical conjugate. In some embodiments, the folate is administered up to 8 hours prior to administering the radiopharmaceutical conjugate. In some embodiments, the folate is administered up to 4 hours prior to administering the radiopharmaceutical conjugate. In some embodiments, the folate is administered 12, 11, 10, 9, 8, 7, 6, 5, 4, 3, 2.5, 2, 1.5, 1, 0.5, or 0.25 hours prior to administering the radiopharmaceutical conjugate. In some embodiments, the folate is administered 12 to 8 hours prior to administering the radiopharmaceutical conjugate. In some embodiments, the folate is administered 10 to 6 hours prior to administering the radiopharmaceutical conjugate. In some embodiments, the folate is administered 8 to 4 hours prior to administering the radiopharmaceutical conjugate. In some embodiments, the folate is administered 6 to 2 hours prior to administering the radiopharmaceutical conjugate. In some embodiments, the folate is administered within 4 hours prior to administering the radiopharmaceutical conjugate. In some embodiments, the folate is administered 12 hours prior to administering the radiopharmaceutical conjugate. In some embodiments, the folate is administered 11 hours prior to administering the radiopharmaceutical conjugate. In some embodiments, the folate is administered 10 hours prior to administering the radiopharmaceutical conjugate. In some embodiments, the folate is administered 9 hours prior to administering the radiopharmaceutical conjugate. In some embodiments, the folate is administered 8 hours prior to administering the radiopharmaceutical conjugate. In some embodiments, the folate is administered 7 hours prior to administering the radiopharmaceutical conjugate. In some embodiments, the folate is administered 6 hours prior to administering the radiopharmaceutical conjugate. In some embodiments, the folate is administered 5 hours prior to administering the radiopharmaceutical conjugate. In some embodiments, the folate is administered 4 hours prior to administering the radiopharmaceutical conjugate. In some embodiments, the folate is administered 3 hours prior to administering the radiopharmaceutical conjugate. In some embodiments, the folate is administered 2.5 hours prior to administering the radiopharmaceutical conjugate. In some embodiments, the folate is administered 2 hours prior to administering the radiopharmaceutical conjugate. In some embodiments, the folate is administered 1.5 hours prior to administering the radiopharmaceutical conjugate. In some embodiments, the folate is administered 1 hour prior to administering the radiopharmaceutical conjugate. In some embodiments, the folate is administered 0.5 hours prior to administering the radiopharmaceutical conjugate. In some embodiments, the folate is administered 0.25 hours prior to administering the radiopharmaceutical conjugate. In some embodiments, the folate is administered in an amount between 1 μg and 1 g. In some embodiments, the folate is administered in an amount between 10 μg and 100 mg. In some embodiments, the folate is administered in an amount between 100 μg and 100 mg. In some embodiments, the folate is administered in an amount between 100 μg and 50 mg. In some embodiments, the folate is administered in an amount between 100 μg and 25 mg. In some embodiments, the folate is administered in an amount between 100 μg and 1 mg. In some embodiments, the folate is administered in an amount between 200 μg and 2 mg. In some embodiments, the folate is administered in an amount between 300 μg and 3 mg. In some embodiments, the folate is administered in an amount between 400 μg and 4 mg. In some embodiments, the folate is administered in an amount between 500 μg and 5 mg. In some embodiments, the folate is administered in an amount between 600 μg and 6 mg. In some embodiments, the folate is administered in an amount between 700 μg and 7 mg. In some embodiments, the folate is administered in an amount between 800 μg and 8 mg. In some embodiments, the folate is administered in an amount between 900 μg and 9 mg. In some embodiments, the folate is administered in an amount between 1 mg and 10 mg. In some embodiments, the folate is administered in an amount between 2 mg and 20 mg. In some embodiments, the folate is administered in an amount between 3 mg and 30 mg. In some embodiments, the folate is administered in an amount between 4 mg and 40 mg. In some embodiments, the folate is administered in an amount between 5 mg and 50 mg. In some embodiments, the folate is administered in an amount of 400 μg, 800 μg, 1 mg, 5 mg, 7.5 mg, 10 mg, or 15 mg. In some embodiments, the folate is folic acid, dihydrofolate (DHF), tetrahydrofolate (THF), 5, 10-methylenetetrahydrofolate (5, 10-MTHF), or 5-methyltetrahydrofolate (5-MTHF). In some embodiments, the folate is folic acid or 5-MTHF. In some embodiments, the folate is folic acid.

Further embodiments of the methods described herein are enumerated in the following clauses:

Clause 1: A method of preventing or reducing the formation of a metastatic bone lesion in a human subject in need thereof, comprising administering to the subject a radiopharmaceutical composition comprising
(a) a compound of Formula (I):

TL-L-R^{M} Formula (I)
or a pharmaceutically acceptable salt thereof, wherein:
TL is a PSMA targeting ligand comprising a urea;
L is a bivalent linking moiety; and
R^{M} is a metal chelator; and
(b) a radionuclide bound to the metal chelator, wherein the radionuclide is ²¹²Pb;
   wherein the human subject has prostate cancer; and
   wherein the radiopharmaceutical conjugate is administered to the human subject in an amount between 50 kBq to 500 MBq per dose.

Clause 2: The method of clause 1, wherein the radiopharmaceutical conjugate comprises a structure of Formula (I-B-1) or Formula (I-B-2):

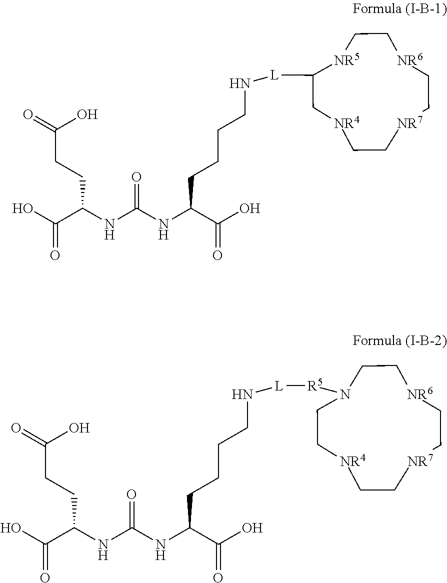

or a pharmaceutically acceptable salt thereof, wherein:
R⁴, R⁵, R⁶, and R⁷ are each independently selected from the group consisting of —C₁₋₃alkylene-C(═O)OR³, and —C₁₋₃alkylene-C(═O)N(R³)₂, wherein each C₁₋₃alkylene is optionally substituted with —C(═O)OH; and
each R³ is independently selected from the group consisting of H and C₁₋₆alkyl;
provided that R⁵ in Formula (I-B-2) is —C₁₋₃alkylene-C(═O)—, wherein the C₁₋₃alkylene is optionally substituted with —C(═O)OH.

Clause 3: The method of clause 1, wherein the compound of Formula (I) is

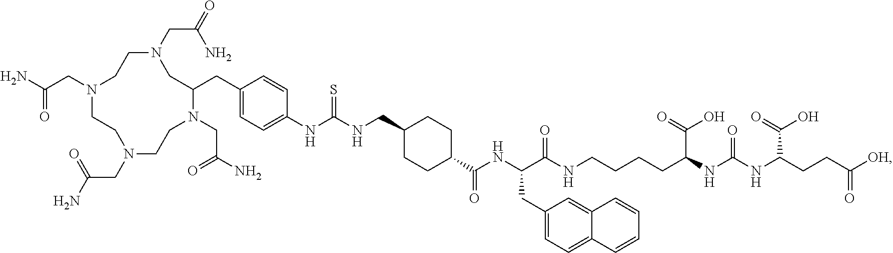

or a pharmaceutically acceptable salt thereof.

Clause 4: The method of clause 1, wherein the compound of Formula (I) is

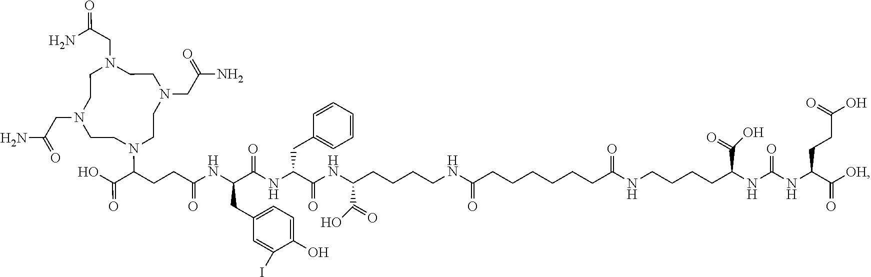 [SMILES: NC(=O)CN1CCN(CC(N)=O)CCN(C(CCC(=O)N[C@H](Cc2ccc(O)c(I)c2)C(=O)N[C@H](Cc2ccccc2)C(=O)N[C@H](CCCCNC(=O)CCCCCCC(=O)NCCCC[C@H](NC(=O)N[C@@H](CCC(=O)O)C(=O)O)C(=O)O)C(=O)O)C(=O)O)CCN(CC(N)=O)CC1]

or a pharmaceutically acceptable salt thereof.

Clause 5: The method of clause 1, wherein the prostate cancer is local and administering the radiopharmaceutical conjugate to the subject prevents or reduces skeletal metastases.

Clause 6: The method of clause 1, wherein the prostate cancer is metastatic, but does not comprise bone lesions, and administering the radiopharmaceutical conjugate to the subject prevents or reduces further skeletal metastases.

Clause 7: The method of clause 1, wherein the radiopharmaceutical conjugate is administered in an amount of about 75 MBq per dose to about 300 MBq per dose.

Clause 8: The method of clause 1, wherein the radiopharmaceutical conjugate is administered in an amount of about 75 MBq per dose, about 100 MBq per dose, about 150 MBq per dose, about 200 MBq per dose, about 250 MBq per dose, or about 300 MBq per dose.

Clause 9: The method of clause 1, wherein the radiopharmaceutical conjugate is administered about once every week, about once every 2 weeks, about once every 3 weeks, about once every 4 weeks, about once every 5 weeks, or about once every 6 weeks.

Clause 10: The method of clause 1, wherein the radiopharmaceutical conjugate is administered 2 times (i.e., for 2 cycles), 3 times, (i.e., for 3 cycles), 4 times, (i.e., 4 cycles), 5 times (i.e., 5 cycles), 6 times (i.e., 6 cycles), 7 times (i.e., 7 cycles), 8 times (i.e., 8 cycles), 9 times (i.e., 9 cycles), 10 times (i.e., 10 cycles), 11 times (i.e., 11 cycles), or 12 times (i.e., 12 cycles).

Clause 11: A method of treating a prostate cancer in a human subject in need thereof, comprising: administering to the human subject a radiopharmaceutical conjugate, wherein the radiopharmaceutical conjugate comprises,
(a) a compound of Formula (I):

TL-L-R^{M} Formula (I)
or a pharmaceutically acceptable salt thereof, wherein:
TL is a PSMA targeting ligand comprising a urea;
L is a bivalent linking moiety; and
R^{M} is a metal chelator; and
(b) a radionuclide bound to the metal chelator, wherein the radionuclide is ²¹²Pb; and wherein the radiopharmaceutical conjugate is administered in combination with folate.

Clause 12: The method of clause 11, wherein the compound has the structure of

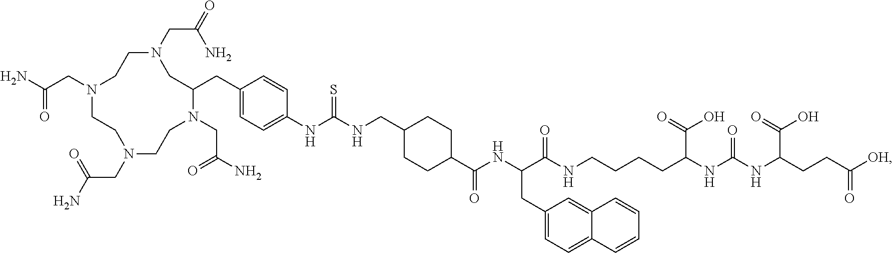 [SMILES: NC(=O)CN1CCN(CC(N)=O)CCN(CC(N)=O)C(Cc2ccc(NC(=S)NCC3CCC(C(=O)NC(Cc4ccc5ccccc5c4)C(=O)NCCCCC(NC(=O)NC(CCC(=O)O)C(=O)O)C(=O)O)CC3)cc2)CN(CC(N)=O)CC1]

or a pharmaceutically acceptable salt thereof.

Clause 13: The method of clause 11, wherein the compound has the structure of

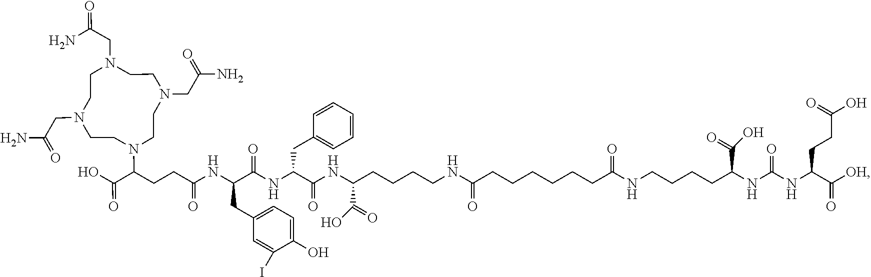 [SMILES: NC(=O)CN1CCN(CC(N)=O)CCN(C(CCC(=O)N[C@H](Cc2ccc(O)c(I)c2)C(=O)N[C@H](Cc2ccccc2)C(=O)N[C@H](CCCCNC(=O)CCCCCCC(=O)NCCCC[C@H](NC(=O)N[C@@H](CCC(=O)O)C(=O)O)C(=O)O)C(=O)O)C(=O)O)CCN(CC(N)=O)CC1]

or a pharmaceutically acceptable salt thereof.

Clause 14: The method of clause 11, wherein the folate is administered prior to, or concurrently with, the radiopharmaceutical conjugate.

Clause 15: The method of clause 11, wherein the folate is administered concurrently with the radiopharmaceutical conjugate.

Clause 16: The method of clause 11, wherein the folate is administered up to 12 hours prior to administering the radiopharmaceutical conjugate.

Clause 17: The method of clause 11, wherein the folate is administered in an amount between 100 μg and 25 mg.

Clause 18: The method of clause 11, wherein the folate is folic acid, dihydrofolate (DHF), tetrahydrofolate (THF), 5, 10-methylenetetrahydrofolate (5, 10-MTHF), or 5-methyltetrahydrofolate (5-MTHF).

Clause 19: The method of clause 11, wherein the radiopharmaceutical conjugate is administered in an amount between 50 kBq to 500 MBq per dose.

Clause 20: The method of clause 11, wherein the radiopharmaceutical conjugate is administered in an amount of about 75 MBq per dose to about 300 MBq per dose.

Clause 21: The method of clause 11, wherein the radiopharmaceutical conjugate is administered about once every week, about once every 2 weeks, about once every 3 weeks, about once every 4 weeks, about once every 5 weeks, or about once every 6 weeks.

Clause 22: The method of clause 11, wherein the radiopharmaceutical conjugate is administered 2 times (i.e., for 2 cycles), 3 times, (i.e., for 3 cycles), 4 times, (i.e., 4 cycles), 5 times (i.e., 5 cycles), 6 times (i.e., 6 cycles), 7 times (i.e., 7 cycles), 8 times (i.e., 8 cycles), 9 times (i.e., 9 cycles), 10 times (i.e., 10 cycles), 11 times (i.e., 11 cycles), or 12 times (i.e., 12 cycles).

Clause 23: A method of treating a prostate cancer in a human subject in need thereof, comprising administering to the human subject a radiopharmaceutical conjugate, wherein the radiopharmaceutical conjugate comprises

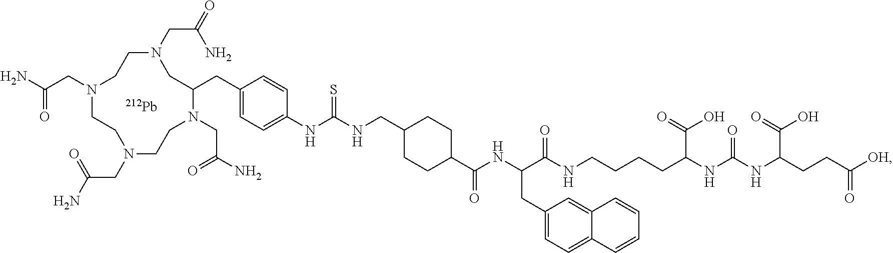

or a pharmaceutically acceptable salt thereof,
wherein the radiopharmaceutical conjugate is administered to the subject about once every week, about once every 2 weeks, about once every 3 weeks, about once every 4 weeks, about once every 5 weeks, or about once every 6 weeks.

Clause 24: The method of clause 23, wherein the radiopharmaceutical conjugate is administered to the subject about once every 3 weeks.

Clause 25: The method of clause 23, wherein the radiopharmaceutical conjugate is administered to the subject about once every 4 weeks.

Clause 26: The method of clause 23, wherein the radiopharmaceutical conjugate is administered to the subject about once every 5 weeks.

Clause 27: The method of clause 23, wherein the radiopharmaceutical conjugate is administered 2 times (i.e., for 2 cycles), 3 times, (i.e., for 3 cycles), 4 times, (i.e., 4 cycles), 5 times (i.e., 5 cycles), 6 times (i.e., 6 cycles), 7 times (i.e., 7 cycles), 8 times (i.e., 8 cycles), 9 times (i.e., 9 cycles), 10 times (i.e., 10 cycles), 11 times (i.e., 11 cycles), or 12 times (i.e., 12 cycles).

Clause 28: The method of clause 23, wherein the radiopharmaceutical conjugate is administered 4 times, 5 times, or 6 times.

Clause 29: The method of clause 23, wherein the radiopharmaceutical conjugate is administered in an amount between 50 kBq to 500 MBq per dose.

Clause 30: The method of clause 23, wherein the radiopharmaceutical conjugate is administered in an amount of about 75 MBq per dose to about 300 MBq per dose.

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

### EXAMPLES

### Example 1: Study Objectives and Overview

### Study Objectives

### Primary Objective

To explore whether the use of single-photon emission computed tomography (SPECT)/computed tomography (CT) gamma camera imaging is feasible for confirming uptake of ²¹²Pb-Compound 1 in various normal organs/tissue and in metastases.

### Secondary Objectives

To determine safety and tolerability of first-in-man exposure of ²¹²Pb-Compound 1.
To study body clearance of ²¹²Pb-Compound 1 by gamma camera imaging and by sample and probe measurements.
Based on visual assessment, localize metastatic lesion(s) with ²¹²Pb-Compound 1 uptake, and compare to localization of lesions detected by PSMA PET imaging.
Based on visual assessment of uptake in normal tissue such as kidneys and salivary glands, to match overlap between the therapeutic and diagnostic images in order to make a go/no-go decision for the Phase 1 dose-escalation part of the study.
To explore any signals of therapeutic effects on disease-related biochemical markers prostate-specific antigen (PSA) and alkaline phosphatase (ALP).

### Methods

The sample size was three participants.

Key inclusion criteria included progressive mCRPC, PSMA-avid lesions demonstrated by PSMA PET/CT, an ECOG performance status 0-2, life expectancy greater than 6 months, and adequate hematopoietic, kidney and liver function.

Study drug administration: a single injection of 10 MBq for ²¹²Pb-Compound 1 was administered for each participant. Administration was by slow bolus IV injection via a 3-way adapter coupled to a conventional isotonic saline solution.
The timepoint of injection of ²¹²Pb-Compound 1 was defined as Day 0, 0 hours.
   Imaging, whole-body probe and blood assessments of clearance were performed at the following timepoints:
Gamma camera imaging (both planar and SPECT/CT) was scheduled 1-3 hours and 16-24 hours post injection.
Clearance was assessed with blood sampling and whole-body probe measurements at the following timepoints: 15 min after dose (+/−15 min window), 1 h after dose (+1 h window), 4 h after dose (+2 h window), 16 h after dose (+8 h window). Full blood and plasma were measured separately to investigate ligand in vivo stability.
   Follow-up: Patients were followed-up for safety and tolerability for 28 days post-administration of ²¹²Pb-Compound 1, with visits on Day 14 and Day 28. Adverse events were reported per CTCAE v5 from time of informed consent until end of follow-up. Patients were followed-up for efficacy biomarkers (PSA, ALP) for 28 days post-administration of ²¹²Pb-Compound 1.

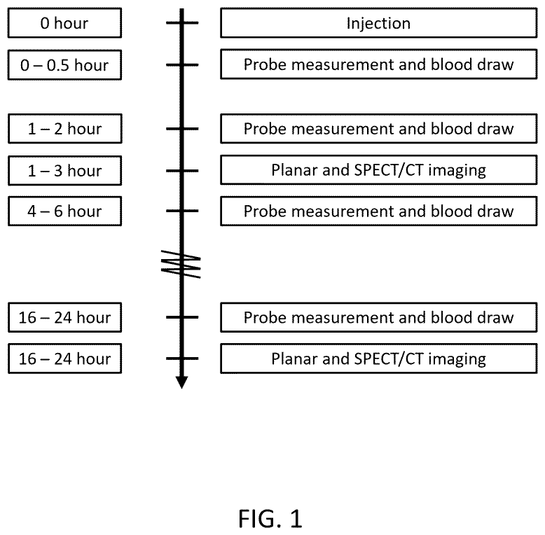

Table 2 depicts the study flow chart. A diagrammatic representation of the timepoints for planar and SPECT imaging, whole-body probe measurements, and blood sample draws is shown in FIG. 1.

TABLE 2
Study Flow-chart

| | Study period | | | | | |
|---|---|---|---|---|---|---|
| | Screening period | Treatment period | | | | |
| | Visit (V) number | | | | | |
| | V1 | V2 | | V3 | V4 | V5 (EOS) |
| | Weeks | | | | | |
| | −4 to 0 | 0 | | 0 | 2 | 4 |
| | Study days | | | | | |
| | | 0 | | | | |
| Clinical Assessments | −28 to 0 days | Pre dose | Post dose | 1 | 14 ± 2 | 28 ± 2 |
| Written Informed consent | X | | | | | |
| Inclusion/Exclusion Criteria | X | | | | | |
| Demographic Data | X | | | | | |
| Medical History | X | | | | | |
| Prior Prostate Cancer Therapies | X | | | | | |
| Physical Examination | X | X | | X | X | X |
| Vital Signs | X | X | | X | X | X |
| ECOG | X | X | | | X | X |
| ¹⁸F-PSMA-PET | X^{a} | | | | | |
| Diagnostic CT or MRI^{b} | X | | | | | |
| 12-lead ECG | X | | | | | |
| Haematology | X | X | | | X | X |
| Biochemistry | X | X | | | X | X |
| Urine analyses (dipstick) | X | X | | | | |
| Whole blood (clearance analysis)^{e} | | | X^{c} | X^{c} | | |
| Administration of ²¹²Pb-Compound 1 | | X | | | | |
| Planar gamma imaging and | | | X^{d} | X^{e} | | |
| SPECT/CT | | | | | | |
| Whole body probe measurements | | X^{f} | X^{f} | X^{f} | | |
| Biomarkers (PSA, ALP) | | X | | | | X |
| Adverse events | | | X | X | X | X |
| Concomitant Medications | | | X | X | X | X |

ALP, alkaline phosphatase; CT, computed tomography; ECG, electrocardiogram; ECOG, The Eastern Cooperative Oncology Group; EOS, end of study; IMP, investigational medicinal product; MRI, magnetic resonance imaging; PET, positron emission tomography; PI, principal investigator; PSA, prostate specific antigen; PSMA, prostate specific membrane antigen; SPECT, single-photon emission computed tomography.
^{a}Diagnostic PSMA-PET no older than six weeks prior to IMP administration. PSMA-PET/CT may be conducted using other approved PSMA-targeted PET radiotracers (including those based on 68Ga).
^{b}Not mandatory, PI's discretion.
^{c}Whole blood samples for body clearance analysis should be taken at the following time-points: 15 min after dose (+/−15 min window), one hour after dose (+1 h window), four hours after dose (+2 h window), 16 hours after dose (+8 h window), to be collected if technically feasible at the site. An additional or replacement sample may be collected within the frame of the existing visit schedule. Such an additional sample should be collected only if it is deemed safe and needed for the technical assessment by the Investigator.
^{d}Within 1-3 hours after IMP administration.
^{e}Within 16-24 hours after IMP administration.
^{f}Non-invasive whole body probe measurements should be taken at the following time-points: predose, 15 min after dose (+/−15 min window), one hour after dose (+1 h window), four hours after dose (+2 h window), 16 hours after dose (+8 h window), to be collected if technically feasible at the site.

### Example 2: Imaging

PSMA-PET/CT imaging

Participants underwent a standard of care diagnostic ¹⁸F-PSMA-PET/CT at baseline (i,e, within six weeks prior to administration of ²¹²Pb-Compound 1).

PSMA-PET/CT was conducted using the radiotracer ¹⁸F-PSMA-1007: dose 2.5 MBq/kg (minimum 200 MBq, maximum 300 MBq) with PET acquisition performed approximately 120 minutes post-injection.

### Gamma Camera Imaging

Non-invasive gamma camera imaging with both planar gamma imaging (whole body scintigraphy) and SPECT/CT was performed at 2 timepoints following injection of ²¹²Pb-Compound 1:
   1-3 hours and 16-24 hours
   The planar gamma camera imaging was acquired immediately before the SEPCT/CT imaging
Performed on a Siemens Symbia Intevo Bold hybrid scanner using a medium energy low penetration (MELP) or high energy (HE) collimator.
The same scanner was used for all acquisitions of all patients in the study.
Scanner room background radiation was measured 0.5 m from the SPECT/CT scanner table, over a time period of one minute, before the participant entered the scanner room. The measurement was repeated 0.5 m from the participant's parotid glands (from each side) and over the kidneys (posterior midline), respectively.
Planar gamma imaging whole body acquisition times were 30-40 minutes
SPECT/CT acquisition involved 2 or 3 bed positions with body contouring orbits starting from the vertex, 20-30 minutes per bed position.
   Two energy windows, 40% at 79 keV and/or 20% at 239 keV.
   Dual scatter windows of 20% for the 79 keV peak centered on 55 keV and 103 keV and 5% for the 239 keV peak centered on 209 keV and 268 keV for scatter correction.
   Attenuation correction based on a low-dose non-enhanced CT.
   Reconstruction parameters: 256×256 matrix, FLASH 3D or xSPECT and iterations x subsets: 15x1, 30x2, 30x4, 30x30.

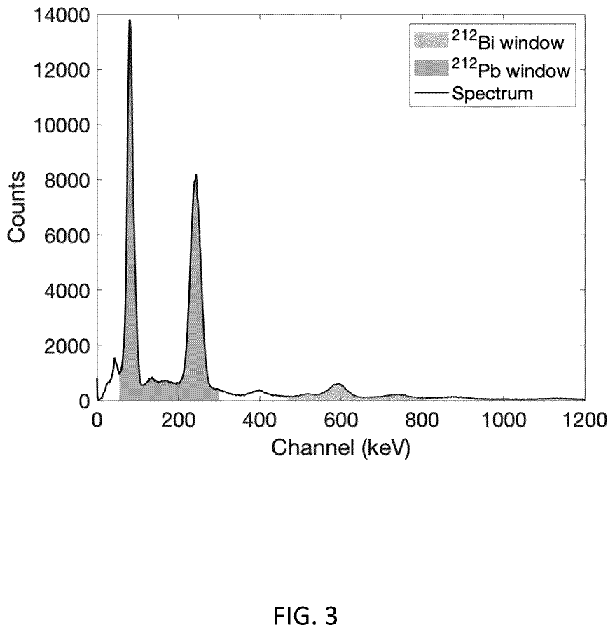

Energy spectra indicating ²¹²Bi and ²¹²Pb windows for imaging is shown in FIG. 3.

### Example 3: Patient Population: Demography, Cancer History and Medical History

Three male patients with progressive mCRPC on standard of care therapies were included in the study. The median age was 81 years at the time of informed consent (range 73-89 years), and mean height and weight were 181.5 m and 84.9 kg respectively. All patients had an ECOG status of 1 at study entry. Patient demographic data are shown in Table 3.

TABLE 3
Demographics

| | Phase 0 Participants | |
|---|---|---|
| Characteristic | (N = 3) | |
| Ethnic Group, n (%) | | |
| White | 3 | (100%) |
| Age at informed consent (years) | | |
| Mean (SD) | 81 | (8.0) |
| Median (Min, Max) | 81 | (73,89) |
| Height | | |
| Mean (SD) | 181.5 | (4.9) |
| Median (Min, Max) | 182 | (178, 185) |
| Body weight (kg) | | |
| Mean (SD) | 84.9 | (22.1) |
| Median (Min, Max) | 86.5 | (62.0, 106) |

ID, identifier; SD, standard deviation.

All patients had stage IV metastatic castration resistant prostate cancer at the time of study entry. In each case this included the presence of locally extensive disease of the prostate and bone metastases. All patients had received prior external beam radiotherapy before enrolment to the clinical trial (see Table 4). No patient received external beam radiation therapy while on study. —P-GP-89-T

TABLE 4
Previous anti-cancer radiotherapy

| Participant | | | Reason for |
|---|---|---|---|
| ID | Start Date | End Date | Ending Therapy |
| NO-01-001 | 22 Oct. 2009 | 25 Nov. 2009 | Radiological |
| | | | progression |
| | NK May 2015 | NK May 2015 | Radiological |
| | | | progression |
| NO-01-002 | 23 Sep. 2015 | NK November 2015 | Completed |
| | 8 Aug. 2019 | 26 Aug. 2019 | Completed |
| | 20 Aug. 2020 | 20 Aug. 2020 | Completed |
| | 20 Mar. 2023 | 31 Mar. 2023 | Completed |
| NO-01-003 | NK 2009 | NK 2009 | Completed |

ID, identifier;

All patients had received prior systemic anticancer therapy before enrolment to the study. One patient had received prior taxane based chemotherapy (docetaxel), whereas two patients had received only prior hormonal based therapies (See Table 5).

TABLE 5
Previous anti-cancer systemic therapy

| | | | | | | | TNM |
|---|---|---|---|---|---|---|---|
| Participant | Type of | | Start | End | Dose | | Stage of |
| ID | Therapy | Description | Date | Date | (mg) | Frequency | Disease |
| NO-01-001 | Hormonal | Bicalutamide | 13 Mar. | 06 Oct. | 150 | Once daily | T3NoM1b |
| | therapy | | 2006 | 2009 | mg | | |
| | | Eulexin | 06 Oct. | 27 Apr. | 250 | Three times | T3NoM1b |
| | | | 2009 | 2015 | mg | a day | |
| | | Zoladex | 06 Mar. | 18 Sep. | 10 | Every 12 | T3NoM1b |
| | | | 2015 | 2015 | mg | weeks | |
| NO-01-002 | Chemotherapy | Docetaxel | 1 Feb. | 25 Apr. | 150 | Every third | T3NoM1b |
| | | | 2016 | 2016 | mg | week | |
| | | Docetaxel-21 | 11 Jan. | 11 Jan. | 170 | Once due to | T3NoM1b |
| | | | 2016 | 2016 | mg | side effects | |
| NO-01-003 | Hormonal | Bicalutamide | NK 2015 | 13 Feb. | 150 | Once daily | T3NxM1b |
| | therapy | | | 2020 | mg | | |

ID, identifier;

All patients enrolled had prior medical conditions reported at the time of informed consent (i.e. medical history) as described in Table 6.

TABLE 6
Medical history/concomitant diseases

| | | Phase 0 | |
|---|---|---|---|
| | | patients | |
| System Organ Class (SOC) | Preferred Term (PT) | n | % |
| Cardiac disorders | Atrial fibrillation | 1 | 33% |
| | Myocardial ischaemia | 1 | 33% |
| | Any preferred term | 2 | 67% |
| Gastrointestinal disorders | Hiatus hernia | 1 | 33% |
| | Any preferred term | 1 | 33% |
| Injury, poisoning and procedural | Spinal compression | 1 | 33% |
| complications | fracture | | |
| | Any preferred term | 1 | 33% |
| Metabolism and nutrition | Folate deficiency | 1 | 33% |
| disorders | Hypercholesterolaemia | 1 | 33% |
| | Any preferred term | 2 | 67% |
| Musculoskeletal and connective | Bone pain | 1 | 33% |
| tissue disorders | | | |
| | Osteoporosis | 1 | 33% |
| | Spinal stenosis | 1 | 33% |
| | Any preferred term | 2 | 67% |
| Nervous system disorders | Polyneuropathy | 1 | 33% |
| | Any preferred term | 1 | 33% |
| Renal and urinary disorders | Lower urinary tract | 1 | 33% |
| | symptoms | | |
| | Any preferred term | 1 | 33% |
| Surgical and medical procedures | Artificial urinary | 1 | 33% |
| | sphincter implant | | |
| | Any preferred term | 1 | 33% |
| Vascular disorders | Hypertension | 2 | 67% |
| | Any preferred term | 2 | 67% |
| Any system organ class | Any preferred term | 3 | 100% |

All patients were reported to receive concomitant medication during the study as described in Table 7. All patients received hormonal therapy for mCRPC during the study, as permitted per protocol (e.g. enzalutamide, goserelin and leuprorelin). During the study, no patients received concomitant chemotherapy, biological therapies, immuno-oncology agents or radiation therapies.

TABLE 7
Concomitant Medications

| | Number (%) of |
|---|---|
| ATC Level 2/ATC Level 5 | Participants (N = 3) |
| A02 Drugs for acid related disorders | |
| Esomeprazole | 1 (33) |
| A06 Drugs for constipation | |
| Macrogol, combinations | 1 (33) |
| Sodium picosulfate | 1 (33) |
| A11 Vitamins | |
| Vitamin D and analogues | 1 (33) |
| A12 Mineral supplements | |
| Calcium, combinations with vitamin d | 2 (67) |
| and/or other drugs | |
| B01 Antithrombotic agents | |
| Acetylsalicylic acid | 1 (33) |
| Warfarin | 1 (33) |
| B03 Antianemic preparations | |
| Cyanocobalamin | 1 (33) |
| Folic acid | 1 (33) |
| C09 Agents acting on the renin- | |
| angiotensin system | |
| Candesartan | 1 (33) |
| Losartan and diuretics | 1 (33) |
| Valsartan, amlodipine and | 1 (33) |
| hydrochlorothiazide | |
| C10 Lipid modifying agents | |
| Atorvastatin | 2 (67) |
| Ezetimibe | 1 (33) |
| L02 Endocrine therapy | |
| Enzalutamide | 3 (100) |
| Goserelin | 2 (67) |
| Leuprorelin | 1 (33) |
| M05 Drugs for treatment of | |
| bone diseases | |
| Denosumab | 1 (33) |
| Zoledronic acid | 1 (33) |
| N02 Analgesics | |
| Morphine | 1 (33) |
| Paracetamol | 1 (33) |
| R06 Antihistamines for systemic use | |
| Alimemazine | 1 (33) |

### Example 4: Administration of Investigational Medical Product (IMP) ²¹²Pb-Compound 1

All patients 3 consented for the clinical trial subsequently received a single administration of 9.4+0.3 MBq ²¹²Pb-Compound 1 by bolus intravenous injection, as described in Table 8.

TABLE 8
IMP Administration

| | IMP dose | IMP | | | IMP | IMP |
|---|---|---|---|---|---|---|
| Participant | prepared | preparation | Start | Stop | infusion | left in |
| ID | (MBq) | time/Date | time | time | time (min) | syringe |
| NO-01- | 9.99 | 15:22/30 | 15:49 | 15:50 | 1 | 0.07 |
| 001 | | Mar. 2023 | | | | |
| NO-01- | 9.72 | 15:17/8 | 15:36 | 15:37 | 1 | 0.04 |
| 002 | | Jun. 2023 | | | | |
| NO-01- | 9.25 | 14:16/11 | 14:31 | 14:32 | 1 | 0.06 |
| 003 | | May 2023 | | | | |

### Example 5: Safety of Investigational Medical Product (IMP)²¹²Pb-Compound 1

All 3 patients consented and administered ²¹²Pb-Compound 1 subsequently completed the planned safety follow up period (no withdrawals from the clinical trial).

The Eastern Cooperative Oncology Group (ECOG) performance status of all patients remained unchanged from baseline to end of study visit. Each patient had an ECOG performance score of 1 at all visits evaluated (see Table 9).

All 3 patients experienced at least 1 treatment emergent adverse event (TEAE) during the clinical trial, as shown in Tables 9-11. All TEAEs reported were CTCAE (v5) Grade 1 or 2; all TEAEs were considered by the Investigator as probably unrelated to ²¹²Pb-Compound 1. No adverse reactions, serious adverse events (SAEs), suspected unexpected serious adverse events (SUSARs) or deaths were reported.

In addition, there were no clinically significant differences or trends reported in hematology, biochemistry and urinalysis from baseline. This included the kidney and liver function parameters alanine aminotransferase (ALT), aspartate aminotransferase (AST), bilirubin, albumin, estimated glomerular filtration rate (egfr) and creatinine. See Table 12 for a summary of safety laboratory values.

No safety concerns arose from the clinical laboratory data, vital signs or any other observations related to safety.

Table 9 provides a listing of the ECOG status of each study participant for each study visit (screening to end of follow up).

TABLE 9
ECOG Performance Status

| Participant | | | Performance |
|---|---|---|---|
| ID | Visit | Date | status (ECOG) |
| NO-01-001 | Screening | 2023 Mar. 6 | 1 |
| | (Day −28 to −1) | | |
| | Visit 2 (Day 0) | 2023 Mar. 30 | 1 |
| | Visit 4 | 2023 Apr. 13 | 1 |
| | Visit 5 | 2023 Apr. 27 | 1 |
| NO-01-002 | Screening | 2023 Apr. 27 | 1 |
| | (Day −28 to −1) | | |
| | Visit 2 (Day 0) | 2023 Jun. 8 | 1 |
| | Visit 4 | 2023 Jun. 22 | 1 |
| | Visit 5 | 2023 Jul. 6 | 1 |
| NO-01-003 | Screening | 2023 Apr. 28 | 1 |
| | (Day −28 to −1) | | |
| | Visit 2 (Day 0) | 2023 May 11 | 1 |
| | Visit 4 | 2023 May 25 | 1 |
| | Visit 5 | 2023 Jun. 8 | 1 |

A summary of the reported TEAEs in the study participants is included in Table 10.

TABLE 10
Number and Proportion of Participants with TEAEs

| | Number (%) of | |
|---|---|---|
| | Participants | |
| | (N = 3) | |
| Number (%) of patients with any adverse event | 3 | (100) |
| Number (%) of patients with 1 adverse event | 3 | (100) |
| Number (%) of patients with 2 adverse events | 2 | (67) |
| Number (%) of patients with ≥3 adverse events | 1 | (33) |
| Number (%) of patients with any possibly related | 0 | (0) |
| adverse event (excluded unrelated) | | |
| Number (%) of patients with serious adverse events | 0 | (0) |

TEAEs are summarized by system organ class (SOC) and preferred term (PT) in Table 11. None of the TEAE PTs were reported in more than one participant. Gastrointestinal disorder was the only SOC for which TEAEs were reported for more than one participant.

TABLE 11
Number and Proportion of Participants with
TEAEs by body system and preferred term

| | Number (%) of |
|---|---|
| System Organ Class/Preferred Term | Participants (N = 3) |
| Injury, poisoning and procedural | 1 (33) |
| complications | |
| Tooth fracture | 1 (33) |
| Gastrointestinal disorders | 3 (33) |
| Abdominal pain | 1 (33) |
| Constipation | 1 (33) |
| Vomiting | 1 (33) |
| General disorders and administration | 1 (33) |
| site conditions | |
| Localized oedema | 1 (33) |
| Skin and subcutaneous tissue disorders | 1 (33) |
| Erythema | 1 (33) |

All reported TEAEs were CTCAE Grade 1, with the exception of abdominal pain which was Common Terminology Criteria for Adverse Events (CTCAE) Grade 2. All reported TEAEs were considered by the Investigator as probably unrelated to study intervention (see Table 12). For laboratory parameter measurements over for each study visit, individual measurements of hematological and clinical chemistry parameters were within their normal reference ranges and variation between visits not considered clinically significant.

TABLE 12
Number of TEAEs by Severity and Relationship to Study Drug by body system and preferred term

| | | | | | | | | Relationship to treatment | | | | | | | | | |
|---|---|---|---|---|---|---|---|---|---|---|---|---|---|---|---|---|---|
| | | CTCAE grade (v5) | | | | | | | | Probably | | Possibly | | Probably | | Not | |
| System Organ | Preferred | 1 | | 2 | | 3-5 | | Unrelated | | unrelated | | related | | related | | assessable | |
| Class (SOC) | term (PT) | n | % | n | % | n | % | n | % | n | % | n | % | n | % | n | % |
| Injury, | | 1 | (33) | 0 | 0% | 0 | 0% | 0 | 0% | 1 | 33% | 0 | 0% | 0 | 0% | 0 | 0% |
| poisoning and | | | | | | | | | | | | | | | | | |
| procedural | | 1 | | | | | | | | | | | | | | | |
| complications | | | | | | | | | | | | | | | | | |
| | Tooth fracture | 1 | 33% | 0 | 0% | 0 | 0% | 0 | 0% | 1 | 33% | 0 | 0% | 0 | 0% | 0 | 0% |
| Gastrointestinal | | 2 | 67% | 1 | 33% | 0 | 0% | 0 | 0% | 3 | 100% | 0 | 0% | 0 | 0% | 0 | 0% |
| disorders | | | | | | | | | | | | | | | | | |
| | Abdominal pain | 0 | 0% | 1 | 33% | 0 | 0% | 0 | 0% | 1 | 33% | 0 | 0% | 0 | 0% | 0 | 0% |
| | Constipation | 1 | 33% | 0 | 0% | 0 | 0% | 0 | 0% | 1 | 33% | 0 | 0% | 0 | 0% | 0 | 0% |
| | Vomiting | 1 | 33% | 0 | 0% | 0 | 0% | 0 | 0% | 1 | 33% | 0 | 0% | 0 | 0% | 0 | 0% |
| General | | 1 | 33% | 0 | 0% | 0 | 0% | 0 | 0% | 1 | 33% | 0 | 0% | 0 | 0% | 0 | 0% |
| disorders and | | | | | | | | | | | | | | | | | |
| administration | | | | | | | | | | | | | | | | | |
| site conditions | | | | | | | | | | | | | | | | | |
| | Localised oedema | 1 | 33% | 0 | 0% | 0 | 0% | 0 | 0% | 1 | 33% | 0 | 0% | 0 | 0% | 0 | 0% |
| Skin and | | 1 | 33% | 0 | 0% | 0 | 0% | 0 | 0% | 1 | 33% | 0 | 0% | 0 | 0% | 0 | 0% |
| subcutaneous | | | | | | | | | | | | | | | | | |
| tissue disorders | | | | | | | | | | | | | | | | | |
| | Erythema | 1 | 33% | 0 | 0% | 0 | 0% | 0 | 0% | 1 | 33% | 0 | 0% | 0 | 0% | 0 | 0% |

### Example 6: Imaging Assessments

All 3 patients completed all planned study imaging procedures for assessment of biodistribution and clearance: PSMA-PET/CT scan at baseline, gamma camera imaging (SPECT/CT and planar gamma imaging) on Day 0 and Day 1 following ²¹²Pb-Compound 1 administration.

### Baseline PSMA-PET CT

Each patient received a baseline PSMA-PET/CT evaluation utilizing an F18-PSMA-PET tracer during the screening period (i.e. prior to ²¹²Pb-Compound 1 administration).

Normal Tissue: PSMA-PET standard uptake values (SUVs) for normal tissues are summarized in Table 13. The pattern of PSMA-PET CT tracer uptake in normal organs is within expectations in each case; with notable uptake in the salivary glands (SUVmax range 20.06-30.7), kidneys (SUVmax range 18.7-35.2) and liver (SUVmax range 14.5-25.5).

Metastatic Lesions: Metastatic lesions were observed for all three participants at screening (<20 lesions each). PSMA-PET standard uptake values (SUVs) for metastatic lesions are summarized in Table 16. All 3 patients had at least three PSMA-avid metastatic lesions, with a wide range of F18-PSMA-PET uptake values with SUVmax of 10.1-77.4. For Participant NO-01-001 lesions were located in bone and viscera, for Participant NO 01 002 lesions were located in bone, and for Participant NO-01-003 lesions were located in bone and lymph node.

²¹²mPb-Compound 1 Gamma Imaging: Planar and SPECT CT

All patients received both planar imaging and SPECT/CT scans at the 1 hour and 16 hour timepoints post administration of ²¹²Pb-Compound 1. Note that as gamma imaging of ²¹²Pb is exploratory, the acquisition parameters (i.e., scan speeds, acquisition times etc.) were optimized during the clinical study between imaging the individual patients.

Normal Tissue: Visual assessment of uptake of ²¹²Pb-Compound 1 in normal tissues was defined as uptake clearly distinguishable from adjacent tissue.

### Planar Gamma Imaging

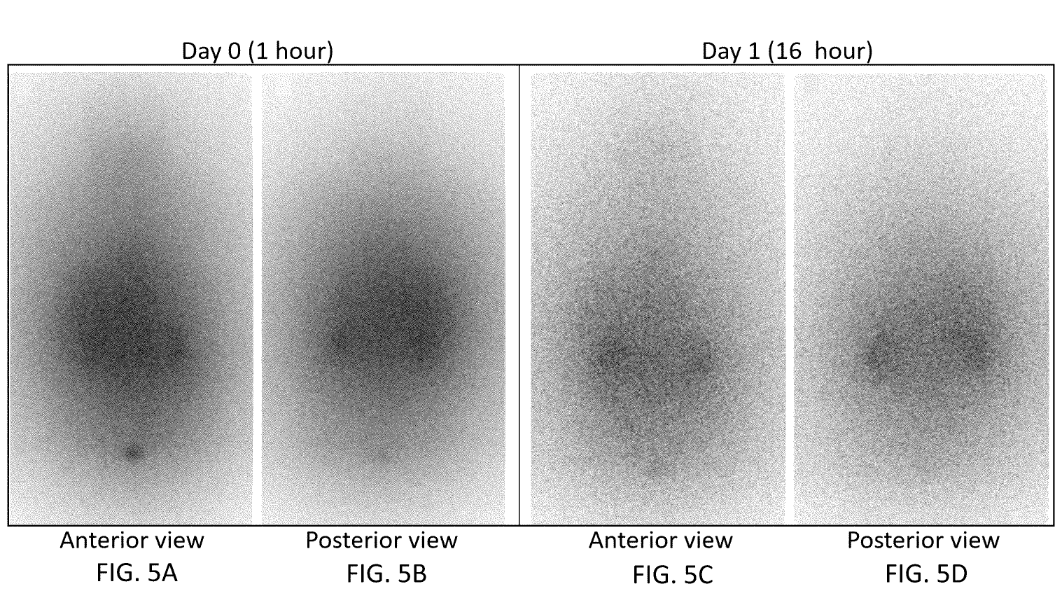

Visual assessment of planar gamma imaging indicated ²¹²Pb-Compound 1 uptake by the kidneys for all three participants at both time points, by the liver in one participant at the 1 hr time point, and by the urinary bladder (including contents) for two participants at both time points.
There was no visualization of the salivary glands indicating ²¹²Pb-Compound 1 uptake on planar gamma imaging in any of the 3 patients at any timepoint.
Visual assessment of ²¹²Pb-Compound 1 by planar gamma imaging in normal tissues in comparison to the liver is presented in Table 14.
Visual assessment of ²¹²Pb-Compound 1 in participant NO-01-002 in normal tissue by planar gamma imaging is shown in FIG. 5A (anterior view 1 hour post injection), FIG. 5B (posterior view 1 hour post injection), FIG. 5C (anterior view 16 hours post injection), and FIG. 5D (posterior view 16 hours post injection).

### SPECT/CT

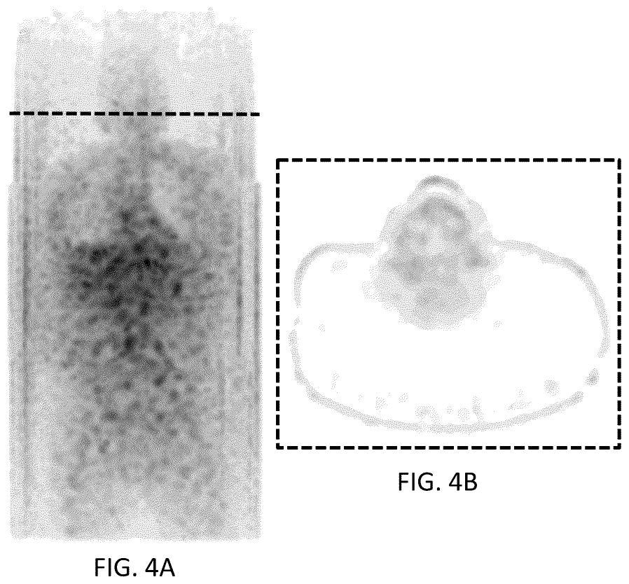

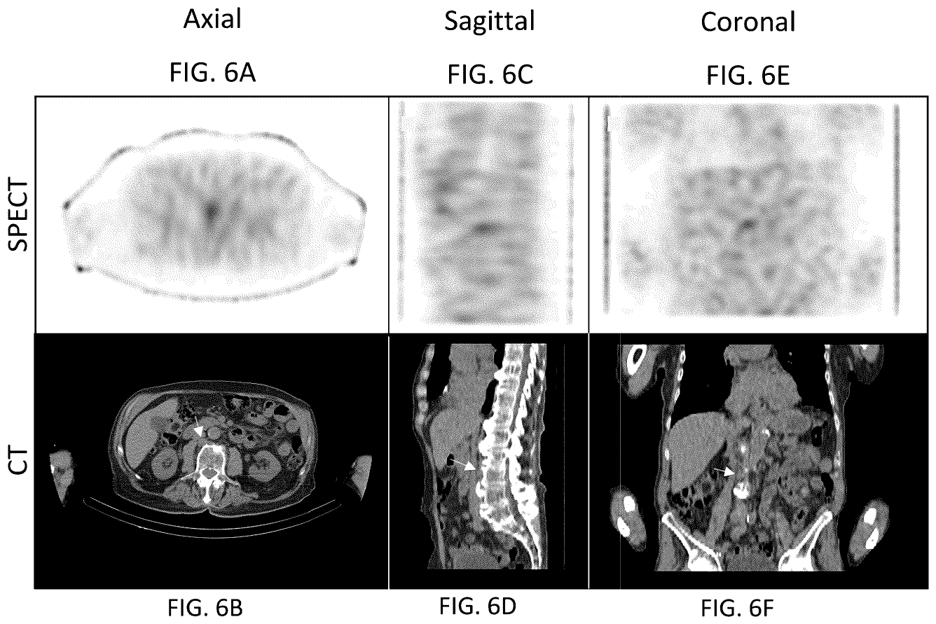

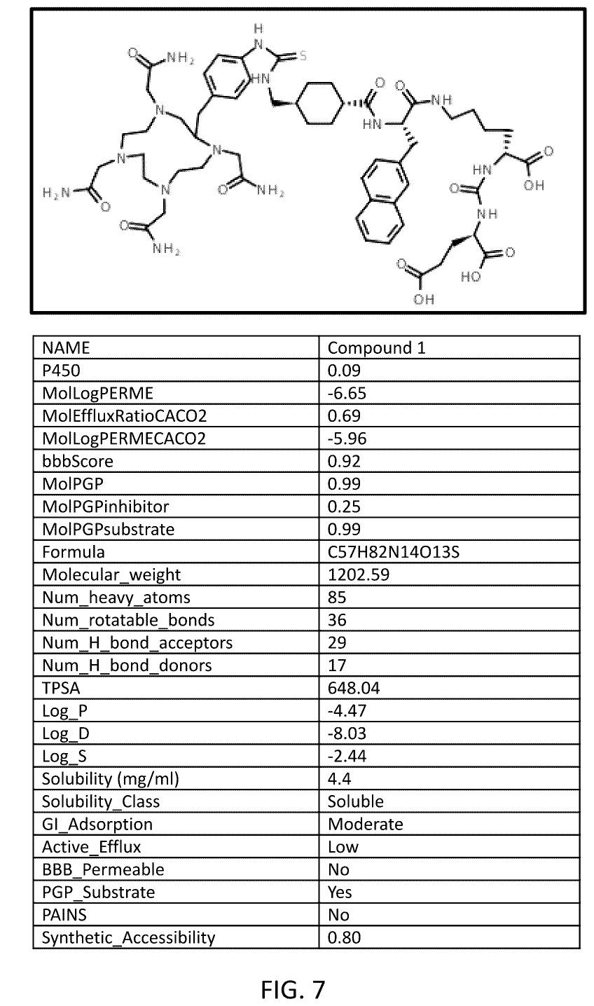

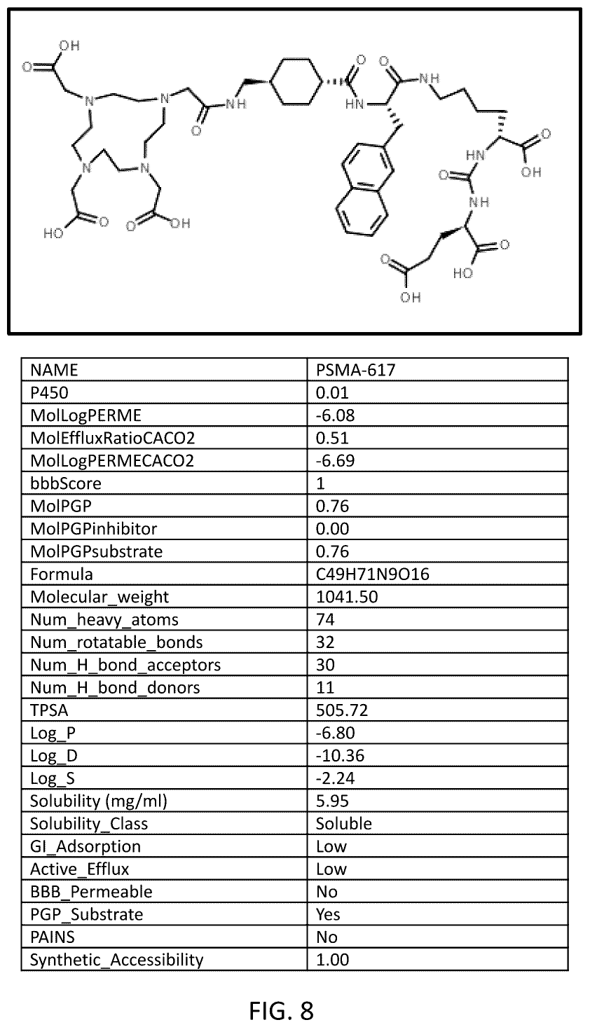

Visual assessment of SPECT/CT images indicated ²¹²Pb-Compound 1 uptake by the kidneys for all three participants for at least one of the time points, by the liver in one participant at one time point, in the blood pool for two participants for at least one of the time points, and in the urinary bladder (including contents) for one participant at one time point.
There was no visualization of the salivary glands indicating ²¹²Pb-Compound 1 uptake on SPECT/CT in any of the 3 patients at any timepoint.
Visual assessment of ²¹²Pb-Compound 1 by SPECT/CT in normal tissues in comparison to the liver is presented in Table 15.
Visual assessment of ²¹²Pb-Compound 1 in participant NO-01-001 in normal tissue by SPECT/CT is shown in FIG. 4A (whole body MIP, anterior view) and FIG. 4B (axial section through the cranium at the level of the parotid glands). ²¹²Pb-Compound 1 uptake could not be visualized in the parotid glands.
Visual assessment of ²¹²Pb-Compound 1 in participant NO-01-003 in the abdominal retrocaval lymph node is shown in FIG. 6, including paired SPECT (summed) scans of the axial section (FIG. 6A), sagittal section (FIG. 6C), and coronal section (FIG. 6E), and non-contrast enhanced CT scans of the axial section (FIG. 6B), sagittal section (FIG. 6D), and coronal section (FIG. 6F). The retrocaval lymph node metastasis is indicated with white arrows on the CT images, and the areas with increased ²¹²Pb-Compound 1 uptake (darker regions) are shown in the SPECT images.

Metastatic Lesions: Visual assessment of SPECT/CT images indicated ²¹²Pb-Compound 1 uptake at the 1 hour time point for one lesion in Participant NO-01-003, a retrocaval lymph node; 11 mm in short axis and 16 mm in longest diameter. This lesion was the most PSMA-avid lesion for any participant, with the highest SUV mean and max demonstrated on PSMA-PET (77.4 Bq/mL and 45 Bq/mL, respectively).

²¹²Pb-Compound 1 was not visualized for any other lesion, at any timepoint, by SPECT/CT or planar gamma imaging. Visual assessment of ²¹²Pb-Compound 1 by planar gamma imaging and SPECT/CT in metastatic lesions in comparison to the liver is presented in Table 17.

TABLE 13
F18-PSMA-PET: Normal Tissue Evaluation at Screening

| | | | | | | | | | | | Small | | | | | | | |
|---|---|---|---|---|---|---|---|---|---|---|---|---|---|---|---|---|---|---|
| | | | | | | | | | | | Intestine, | | | | Bladder, | | | |
| | | | Salivary | | | | | | | | including | | | | including | | Bone | |
| | | | gland | | Kidneys | | Liver | | Spleen | | contents | | Blood pool | | contents | | Marrow | |
| | How | | SUV | SUV | SUV | SUV | SUV | SUV | SUV | SUV | SUV | SUV | SUV | SUV | SUV | SUV | SUV | SUV |
| | many | | max | mean | max | mean | max | mean | max | mean | max | mean | max | mean | max | mean | max | mean |
| Participant | lesions | Regional | Bq/ | Bq/ | Bq/ | Bq/ | Bq/ | Bq/ | Bq/ | Bq/ | Bq/ | Bq/ | Bq/ | Bq/ | Bq/ | Bq/ | Bq/ | Bq/ |
| Id | observed? | locations | mL | mL | mL | mL | mL | mL | mL | mL | mL | mL | mL | mL | mL | mL | mL | mL |
| NO-01- | </=20 | Bone, | 30.7 | 18.5 | 35.2 | 19.2 | 14.5 | 11.3 | 16.2 | 12.6 | 7.1 | 5.4 | 1.2 | 0.7 | 8.3 | 6.2 | 1.8 | 1.3 |
| 001 | | Viscera | | | | | | | | | | | | | | | | |
| NO-01- | </=20 | Bone | 20.6 | 13 | 26.8 | 16.1 | 25.5 | 21.8 | 7.2 | 6.1 | 22.5 | 17.1 | 0.8 | 0.6 | 2.8 | 2.3 | 1.3 | 0.9 |
| 002 | | | | | | | | | | | | | | | | | | |
| NO-01- | </=20 | Lymph | 30.7 | 19.8 | 18.7 | 11.4 | 14.8 | 12.5 | 15.3 | 13.7 | 22 | 16.1 | 1.3 | 1.1 | 3 | 2.3 | 1 | 0.7 |
| 003 | | Node, | | | | | | | | | | | | | | | | |
| | | Bone | | | | | | | | | | | | | | | | |

TABLE 14
Gamma Imaging (Planar): Normal Tissue Evaluation

| | | | | | Salivary gland | | Kidneys | | | Spleen | |
|---|---|---|---|---|---|---|---|---|---|---|---|
| | | | | | | Visual | | Visual | Liver | | Visual |
| | | | | | Did visual | assessment | Did visual | assessment | Did visual | Did visual | assessment |
| | | | Relative | | assessment | of ²¹²Pb- | assessment | of ²¹²Pb- | assessment | assessment | of ²¹²Pb- |
| | | | time of | Scan | indicate | Compound 1 | indicate | Compound 1 | indicate | indicate | Compound 1 |
| | | | scan to | speed | ²¹²Pb- | uptake in | ²¹²Pb- | uptake in | ²¹²Pb- | ²¹²Pb- | uptake in |
| Participant | Time | | dose | (cm/ | Compound 1 | comparison | Compound 1 | comparison | Compound 1 | Compound 1 | comparison |
| Id | point | | (H.M) | min) | uptake? | to liver? | uptake? | to liver? | uptake? | uptake? | to liver? |
| NO-01-001 | 1 | h | 1.59 | 7 | No | | Yes | Greater than | No | No | |
| | 16 | h | 17.44 | 5 | No | | Yes | Greater than | No | No | |
| NO-01-002 | 1 | h | 2.06 | 3 | No | | Yes | Greater than | No | No | |
| | 16 | h | 17.54 | 3 | No | | Yes | Equal to | No | No | |
| NO-01-003 | 1 | h | 2.00 | 4 | No | | Yes | Equal to | Yes | No | |
| | 16 | h | 19.32 | 3 | No | | Yes | Greater than | No | No | |

| Small Intestine, | | | | Bladder, | | | |
|---|---|---|---|---|---|---|---|
| including contents | | Blood pool | | including contents | | Bone Marrow | |
| | Visual | | Visual | | Visual | | Visual |
| Did visual | assessment | Did visual | assessment | Did visual | assessment | Did visual | assessment |
| assessment | of ²¹²Pb- | assessment | of ²¹²Pb- | assessment | of ²¹²Pb- | assessment | of ²¹²Pb- |
| indicate | Compound 1 | indicate | Compound 1 | indicate | Compound 1 | indicate | Compound 1 |
| ²¹²Pb- | uptake in | ²¹²Pb- | uptake in | ²¹²Pb- | uptake in | ²¹²Pb- | uptake in |
| Compound 1 | comparison | Compound 1 | comparison | Compound 1 | comparison | Compound 1 | comparison |
| uptake? | to liver? | uptake? | to liver? | uptake? | to liver? | uptake? | to liver? |
| No | | No | | No | | No | |
| No | | No | | No | | No | |
| No | | No | | Yes | Equal to | No | |
| No | | No | | Yes | Equal to | No | |
| No | | No | | Yes | Equal to | No | |
| No | | No | | Yes | Equal to | No | |

TABLE 15
Gamma Imaging (SPECT/CT): Normal Tissue Evaluation

| | | | | Salivary gland | | Kidneys | | | Spleen | |
|---|---|---|---|---|---|---|---|---|---|---|
| | | | | | Visual | | Visual | Liver | | Visual |
| | | | | Did visual | assessment | Did visual | assessment | Did visual | Did visual | assessment |
| | | | Relative | assessment | of ²¹²Pb- | assessment | of ²¹²Pb- | assessment | assessment | of ²¹²Pb- |
| | | | time of | indicate | Compound 1 | indicate | Compound 1 | indicate | indicate | Compound 1 |
| | | | scan to | ²¹²Pb- | uptake in | ²¹²Pb- | uptake in | ²¹²Pb- | ²¹²Pb- | uptake in |
| Participant | Time | | dose | Compound 1 | comparison | Compound 1 | comparison | Compound 1 | Compound | comparison |
| Id | point | | (H.M) | uptake? | to liver? | uptake? | to liver? | uptake? | 1uptake? | to liver? |
| NO-01-001 | 1 | h | 2.45 | No | | No | | No | No | |
| | 16 | h | 18.34 | Not Applicable | | Yes | Greater than | No | No | |
| NO-01-002 | 1 | h | 3.03 | No | | Yes | Greater than | No | No | |
| | 16 | h | 18.52 | No | | Yes | Greater than | No | No | |
| NO-01-003 | 1 | h | 2.52 | No | | Yes | Equal to | Yes | No | |
| | 16 | h | 20.38 | No | | Yes | Greater than | No | No | |

| Small Intestine, | | | | Bladder, | | | |
|---|---|---|---|---|---|---|---|
| including contents | | Blood pool | | including contents | | Bone Marrow | |
| | Visual | | Visual | | Visual | | Visual |
| Did visual | assessment | Did visual | assessment | Did visual | assessment | Did visual | assessment |
| assessment | of ²¹²Pb- | assessment | of ²¹²Pb- | assessment | of ²¹²Pb- | assessment | of ²¹²Pb- |
| indicate | Compound 1 | indicate | Compound 1 | indicate | Compound 1 | indicate | Compound 1 |
| ²¹²Pb- | uptake in | ²¹²Pb- | uptake in | ²¹²Pb- | uptake in | ²¹²Pb- | uptake in |
| Compound 1 | comparison | Compound 1 | comparison to | Compound 1 | comparison | Compound 1 | comparison to |
| uptake? | to liver? | uptake? | liver? | uptake? | to liver? | uptake? | liver? |
| No | | Yes | Equal to | No | | No | |
| No | | Not Applicable | | No | | No | |
| No | | No | | Yes | Equal to | No | |
| No | | No | | Not Applicable | | No | |
| No | | Yes | Equal to | Not Applicable | | No | |
| No | | No | | No | | No | |

TABLE 16
F18-PSMA-PET: Metastatic Lesion Evaluation

| Participant | | Lesion | Regional | Anatomical | | SUV max | SUV mean | |
|---|---|---|---|---|---|---|---|---|
| ID | | No. | location | location | Size (mm) | Bq/mL | Bq/mL | EANM PSMA-PET score |
| NO-01-001 | Screening | Lesion 1 | Bone | Right pubic bone | 60 | 10.1 | 5.7 | 1 = Equal to or above blood pool |
| | | | | ramus superior | | | | and lower than liver |
| | | Lesion 2 | Visceral | Dorsal bladder | 28 × | 15.9 | 9 | 2 = Equal to or above liver and |
| | | | | wall | 13 × 17 | | | lower than parotid gland |
| | | Lesion 3 | Visceral | Left seminal | 12 × | 10.6 | 6.2 | 1 = Equal to or above blood pool |
| | | | | vesicle | 8 × 10 | | | and lower than liver |
| NO-01-002 | Screening | Lesion 1 | Bone | Sternum | 150 | 28.6 | 14.7 | 2 = Equal to or above liver and |
| | | | | | | | | lower than parotid gland |
| | | Lesion 2 | Bone | Left 3rd rib dorsal | 25 | 30.9 | 20.6 | 3 = Equal to or above parotid gland |
| | | Lesion 3 | Bone | Cervical vertebra | 25 | 18.9 | 11.3 | 2 = Equal to or above liver and |
| | | | | C7 | | | | lower than parotid gland |
| NO-01-003 | Screening | Lesion 1 | Lymph | Retrocaval | 11 × | 77.4 | 45 | 3 = Equal to or above parotid gland |
| | | | node | | 14 × 16 | | | |
| | | Lesion 2 | Bone | Right 4th rib | 40 | 17.4 | 11.1 | 2 = Equal to or above liver and |
| | | | | anterolateral | | | | lower than parotid gland |
| | | Lesion 3 | Bone | Lumbar vertebra | 10 | 13.6 | 8 | 2 = Equal to or above liver and |
| | | | | L3 | | | | lower than parotid gland |

TABLE 17
Gamma Imaging (planar and SPECT/CT): Metastatic Lesion Evaluation

| | | | | | | | Planar | | SPECT-CT | | |
|---|---|---|---|---|---|---|---|---|---|---|---|
| | | | | | | | | Visual | | | Visual |
| | | | | | | | Did visual | assessment | | Did visual | assessment |
| | | | | | | Relative | assessment | of ²¹²Pb- | Relative | assessment | of ²¹²Pb- |
| | | | | | | time of | indicate | Compound 1 | time of | indicate | Compound 1 |
| | | | | | | scan to | ²¹²Pb- | uptake in | scan to | ²¹²Pb- | uptake in |
| Participant | Time | Lesion | Regional | Anatomical | Size | dose | Compound 1 | comparison | dose | Compound 1 | comparison |
| ID | Point | No. | location | location | (mm) | (h.mm) | uptake? | to liver? | (h.mm) | uptake? | to liver? |
| NO-01- | 1 H | Lesion 1 | Bone | Right pubic bone | 60 | 1.59 | No | | 2.45 | No | |
| 001 | | | | ramus superior | | | | | | | |
| | | Lesion 2 | Visceral | Dorsal bladder | 28 × | | No | | 2.45 | No | |
| | | | | wall | 13 × 17 | | | | | | |
| | | Lesion 3 | Visceral | Left seminal | 12 × | | No | | 2.45 | No | |
| | | | | vesicle | 8 × 10 | | | | | | |
| | 16 H | Lesion 1 | Bone | Right pubic bone | 60 | 17.44 | No | | 18.34 | No | |
| | | | | ramus superior | | | | | | | |
| | | Lesion 2 | Visceral | Dorsal bladder | 28 × | | No | | 18.34 | No | |
| | | | | wall | 13 × 17 | | | | | | |
| | | Lesion 3 | Visceral | Left seminal | 12 × | | No | | 18.34 | No | |
| | | | | vesicle | 8 × 10 | | | | | | |
| NO-01- | 1 H | Lesion 1 | Bone | Sternum | 150 | 2.06 | No | | 3.03 | No | |
| 002 | | Lesion 2 | Bone | Left 3rd rib | 25 | | No | | 3.03 | No | |
| | | | | dorsal | | | | | | | |
| | | Lesion 3 | Bone | Cervical | 25 | | No | | 3.03 | No | |
| | | | | vertebra C7 | | | | | | | |
| | 16 H | Lesion 1 | Bone | Sternum | 150 | 17.54 | No | | 18.52 | No | |
| | | Lesion 2 | Bone | Left 3rd rib | 25 | | No | | 18.52 | No | |
| | | | | dorsal | | | | | | | |
| | | Lesion 3 | Bone | Cervical | 25 | | No | | 18.52 | No | |
| | | | | vertebra C7 | | | | | | | |
| NO-01- | 1 H | Lesion 1 | Lymph | Retrocaval | 11 × | 2.00 | No | | 2.52 | Yes | Greater than |
| 003 | | | node | | 14 × 16 | | | | | | |
| | | Lesion 2 | Bone | Right 4th rib | 40 | | No | | 2.52 | No | |
| | | | | anterolateral | | | | | | | |
| | | Lesion 3 | Bone | Lumbar | 10 | | No | | 2.52 | No | |
| | | | | vertebra L3 | | | | | | | |
| | 16 H | Lesion 1 | Lymph | Retrocaval | 11 × | 19.32 | No | | 20.38 | No | |
| | | | node | | 14 × 16 | | | | | | |
| | | Lesion 2 | Bone | Right 4th rib | 40 | | No | | 20.38 | No | |
| | | | | anterolateral | | | | | | | |
| | | Lesion 3 | Bone | Lumbar | 10 | | No | | 20.38 | No | |
| | | | | vertebra L3 | | | | | | | |

### Example 7: Summary of Findings

The primary objective of the study was to explore whether the use of gamma imaging (SPECT/CT and planar gamma imaging) was feasible for confirming uptake of ²¹²Pb-Compound 1 in various normal organs/tissue and in metastases. Further objectives included assessment of safety and tolerability, assessment of body clearance of ²¹²Pb-Compound 1, comparison to PSMA PET imaging, and assessment of the disease related biomarkers PSA and ALP.

Three adult male participants with progressive mCRPC were screened, met the inclusion criteria of the study, received 10 MBq ²¹²Pb-Compound 1 as planned, and completed the study imaging and follow up procedures. ²¹²Pb-Compound 1 was administered to each of the participants without safety concerns from the reported TEAEs, clinical laboratory data, vital signs or any other observations. The study findings support feasibility of gamma imaging for assessing uptake of ²¹²Pb-Compound 1 in various normal organs/tissues and in metastases by visual qualitative assessment.

The most PSMA-avid metastatic lesion, a retrocaval lymph node metastasis with short-axis diameter 11 mm, was visualized with ²¹²Pb-Compound 1 uptake on post-therapy SPECT/CT; however, visualization on planar imaging was not possible. For the other metastatic lesions, visualization of ²¹²Pb-Compound 1 on either SPECT/CT or planar imaging was not clearly demonstrated. This may be due to lower PSMA-expression of these metastases indicated by the lower SUVs on PSMA PET, combined with the low dose of ²¹²Pb-Compound 1 and limitations of gamma imaging described above.

The distribution of ²¹²Pb-Compound 1 observed on planar gamma imaging and SPECT/CT is compatible with the observed distribution of PSMA/PET tracer at screening for most organs, including the kidneys, liver, and urinary bladder, except for the salivary glands which were not detectable.

The absence of observed salivary gland uptake of ²¹²Pb-Compound 1 is notable. Salivary glands express PSMA; both PSMA-targeted PET/CT tracer (as demonstrated at baseline PET/CT imaging) and other PSMA-targeted radiotherapeutic agents typically demonstrate substantial uptake in the salivary glands.

Of note, salivary gland toxicity (xerostomia) is a common for most PSMA-targeting RLTs (e.g. ¹⁷⁷Lu-PSMA-617), and may be the dose limiting toxicity for several of these (e.g. ²²⁵Ac-PSMA-617). Furthermore, xerostomia even at low severity has a strong impact on patient quality of life (e.g. CTCAE grade 1 or 2); whereas higher severity (e.g. CTCAE grade 3) may render patients unable to receive food by mouth.

The observation in the study that on both SPECT/CT and planar gamma imaging that no 212Pb-Compound 1 uptake could be observed to the salivary glands, indicates a lower risk of xerostomia for ²¹²Pb-Compound 1.

### Example 8: Phase 1/2 Study to Investigate the Safety, Tolerability, Pharmacokinetics, Biodistribution and Antitumour Activity of the Alpha-Therapeutic Radioligand ²¹²Pb-Compound 1

This is a Phase 1/2 dose escalation study to investigate if participants with advanced mCRPC can be treated with ²¹²Pb-Compound 1 with an acceptable safety profile and expected positive benefit/risk ratio, and to obtain preliminary data on antitumor activity. The dose escalation design will allow determination of the recommended dose and treatment schedule of ²¹²Pb-Compound 1, for further evaluation and refinement during the dose expansion part of the trial. Furthermore, evaluation will be undertaken of the biodistribution, pharmacokinetics (PK), and body clearance for ²¹²Pb-Compound 1 to support development of this Alpha Radioligand Therapy (ART).

Dose Escalation: A step-wise escalation of the ²¹²Pb dose (MBq) of ²¹²Pb-Compound 1 will be conducted in cohorts of participants with mCRPC who have not received prior treatment with ¹⁷⁷Lu-PSMA, to establish the recommended dose and regimen to be explored in Dose Expansion.

Dose Expansion: The Dose Expansion Groups define mCRPC patient populations with unmet medical need: Group A: participants who have not received prior treatment with ¹⁷⁷Lu-PSMA-617, and Group B: participants who have been treated with ¹⁷⁷Lu-PSMA-617. Participants in Group A will receive the recommended dose and schedule from Dose Escalation. For Group B, a safety run-in cohort will first be opened at one dose level lower to ensure no safety concerns before proceeding to the recommended dose and schedule.

### Example 9: Phase 1/2 study objectives and endpoints

### Objectives: Primary

To determine the safety and tolerability profile of 2′Pb-Compound 1 in participants with mCRPC
Dose Escalation: To determine the Recommended Dose and Schedule for Dose Expansion for ²¹²Pb-Compound 1
Dose Expansion: To determine the Recommended Dose and Schedule for clinical development for ²¹²Pb-Compound 1

### Endpoints: Primary

Incidence and severity of TEAEs (including adverse event of special interest and treatment-emergent serious adverse event)
Incidence of dose limiting toxicity
Preliminary antitumor activity (ORR and PSA₅₀)
Recommended dose and schedule based on safety and preliminary antitumour activity assessments (including incidence of dose limiting toxicities (DLTs) and PSA50 response)
Recommended dose and schedule based on safety and preliminary antitumour efficacy (including incidence and severity of [S]AEs, PSA50 response, and ORR)

### Objectives: Secondary

To determine the preliminary antitumor activity of 2′Pb-Compound 1
To evaluate the antitumor efficacy of ²¹²Pb-Compound 1
To evaluate SPECT/CT gamma camera imaging for ²¹²Pb-Compound 1
To evaluate the pharmacokinetics of 2′Pb-Compound 1

### Endpoints: Secondary

Objective response rate (ORR) (per PCWG3 guidelines)
PSA₅₀ response at >12 weeks
Best overall PSA response
   Radiologic progression-free survival (rPFS) (per Prostate Cancer Working Group 3 (PCWG3) guidelines)
Duration of response (DOR) (per PCWG3 guidelines)
Duration of PSA₅₀ response
Overall survival (OS)
Assessment of uptake in regions of interest (ROIs) in normal tissues and tumor lesions
Blood activity of ²¹²Pb-Compound 1 after single and multiple doses (including Cmax, Cmin, AUC, etc.)

### Objectives: Exploratory

To evaluate early antitumour activity of ²¹²Pb-Compound 1 using PSMA/PET-CT
To further evaluate the antitumour efficacy of ²¹²Pb-Compound 1
To evaluate body clearance of ²¹²Pb-Compound 1
To evaluate biodistribution of ²¹²Pb-Compound 1 by SPECT/CT imaging
To evaluate localisation of lesions detected by SPECT/CT of ²¹²Pb-Compound 1 and baseline PSMA-PET/CT
To investigate PSMA radiotracer uptake in tumour lesions and potential relationship to the antitumour activity of ²¹²Pb-Compound 1
To investigate uptake in tumour lesions of ²¹²Pb-Compound 1 by gamma camera imaging and potential relationship the antitumour activity of ²¹²Pb-Compound 1
To assess potential pharmacodynamic and safety biomarkers for ²¹²Pb-Compound 1
To investigate the safety profile of ²¹²Pb-Compound 1 relative to prior radiation exposure
To assess impact on quality of life for participants with mCRPC receiving ²¹²Pb-Compound 1

### Endpoints: Exploratory

ORR per RECIP 1.0 between baseline to Week 12
Overall ORR using RECIP 1.0 criteria
Overall survival (OS)
Quantification of activity from SPECT/CT imaging
Quantification of activity from whole-body measurements
Urine clearance (up to 48 h)
Assessment of uptake in ROIs in normal tissues
Dosimetry estimations
Compare ²¹²Pb-Compound 1 and PSMA-PET radiotracer uptake in corresponding tumour lesions
Correlation of baseline mean standardised uptake values (SUVs) in tumour lesions from PSMA-PET/CT with antitumour activity (radiological response, PSA50, etc.)
Correlation ROI measurements in tumour lesions from gamma camera imaging with antitumour activity (radiological response, PSA50, etc.)
Various biomarkers (e.g., tumour genomic alterations, circulating safety biomarkers for critical organs, potentially predictive biomarkers, etc)
Assessment of TEAEs with exposure from EBRT and RTLs
Health-related Quality of Life (HRQoL) questionnaires FACT-P and SXI, optionally FACT-RNT

### Example 10: Dose Escalation Cohort

The Dose Escalation part of the study aims to assess the safety and tolerability of ²¹²Pb-Compound 1 and identify the recommended dose and schedule of ²¹²Pb-Compound 1 for the Dose Expansion groups. See the Inclusion and Exclusion Criteria for definition of the participant population for Dose Escalation, including requirements for prior anticancer therapies.

Dose Escalation of ²¹²Pb-Compound 1 will be based on assessment of clinical safety data supported by available PK and tumor response profiles, supported by a Time-to-Event Bayesian Optimal Interval Design (TITE-BOIN) method which includes information over the periods of exposure to ²¹²Pb-Compound 1To enable early dosage optimisation of ²¹²Pb-Compound 1, the Dose Escalation part of the study will aim to evaluate the planned dose levels of ²¹²Pb-Compound 1 and the schedule of treatment (cycle duration).

Dose Escalation will be initiated with a starting dose of 100 MBq of ²¹²Pb for the first cohort of participants. ²¹²Pb dose levels will be increased for subsequent cohorts in a planned stepwise fashion (see Table 18). Dose levels below 100 MBq or increments between the dose levels described in Table 18 may be explored if indicated by clinical efficacy and/or safety data.

The ligand dose for ²¹²Pb-Compound 1 will be in the range of 50-200 μg with no planned escalation. For example, the ligand dose for ²¹²Pb-Compound 1 will be in the range of 50-100 μg (no planned escalation).

TABLE 18
Exemplary ²¹²Pb Dose Escalation Levels

| | | | |
|---|---|---|---|
| Dose | ²¹²Pb | % Change from | No. Treatment cycles |

| Level | Dose | previous Level | Planned | Additional^{a} |
|---|---|---|---|---|
| 1 | 100 MBq | NA | 4 | +2 |
| 2 | 150 MBq | 50% | 4 | +2 |
| 3 | 200 MBq | 33% | 4 | +2 |
| −1^{b} | 75 MBq | −25% | 4 | +2 |

²¹²Pb, lead-212; NA, not applicable; No., number.
^{a}Individual participants may receive an additional two treatment cycles after the four planned if there is clinical benefit in the opinion of the Investigator, and safety criteria are met.
^{b}Dose Level −1 may be explored if indicated by clinical data (efficacy and/or safety). Reference for % change for Dose Level −1 is relative to Dose Level 1.

Dose Escalation will be initiated with ²¹²Pb-Compound 1 administered by slow bolus injection on Day 1 of a 6-week (42-day) cycle. Study treatment administration after Cycle 1 may be delayed by up to an additional six weeks if required to manage treatment-emergent adverse events (TEAEs). Participants may only receive study treatment after Cycle 1 if they meet all safety criteria required to continue, have not progressed according to the Prostate Cancer Working Group 3 (PCWG3) guidelines, and after individual positive benefit/risk assessment by the Investigator and Sponsor.

Four cycles of study treatment are planned. Individual participants may continue up to a maximum of 6 treatment cycles provided they demonstrate all of the following:
Clinical benefit in the opinion of the Investigator
A radiographic response, and/or a PSA50 response, and/or clinical improvement
No progressive disease per PCWG3 criteria
No acute toxicity greater than CTCAE Grade 1. Exception may be made for chronic, stabilized Grade 2 events, which may have contribution from past therapies or the underling disease (e.g. alopecia, neuropathy, fatigue, anorexia, nail changes, etc.)

Cycle Optimization: During Dose Escalation, dependent on the safety and efficacy profile of ²¹²Pb-Compound 1, additional cohorts may be opened to assess optimal cycle duration, including shorter cycle periods (e.g., three or four weeks). Cohorts to assess cycle optimization may be opened in parallel to assessing dose level escalation.

### Dose Escalation Cohorts and Process

Participants will be assigned to cohorts at ²¹²Pb dose levels (per the Cohort Management Plan). In the first ²¹²Pb dose level cohort there will be an interval of at least 7 days between the first and second participants' first dose, and an interval of at least 1 day between the first dose for subsequent participants. In all subsequent cohorts, there will be an interval of at least 1 day between the first and second participants' first dose; however, if a DLT occurs in the first week of treatment for a participant in any cohort, all subsequent cohorts will apply an interval of at least seven days between the first and second participants' first dose as an additional precaution.

The TITE-BOIN approach (Yuan Y, Lin R, Li D, Nie L, Warren K E, “Time-to-event Bayesian optimal interval design to accelerate phase I trials,” Clin Cancer Res. 2018; 24(20):4921-30. doi:10.1158/1078-0432.CCR-18-0246) with a target DLT rate of 30% will be used to guide the decision on the next dose level/schedule that can be taken. If the observed DLT rate at the current dose is <0.236, escalate the dose to the next higher dose level. If the observed DLT rate at the current dose is >0.359, de-escalate the dose to the next lower dose level. If the observed DLT rate at the current dose is between 0.236 and 0.359 then the current dose is maintained.

Initially, three participants will be treated in each Dose Escalation cohort, and a minimum of two participants will have to be DLT-evaluable prior to adjusting dose or dosing schedule. To be considered DLT-evaluable, a participant is required to receive at least 80% of the planned dose at Cycle 1 Day 1 and will have either completed the ‘DLT Observation Period’ of six weeks in the absence of DLT, or have experienced a DLT. Participants who discontinue during the first six weeks due to any reason other than a DLT, and participants who received less than 80% of the planned dose will not be considered evaluable, and will be replaced. 12271 The Phase 1 trial will commence at 100 MBq administered on Day 1 of a 6-week cycle to ensure participant safety. Initially, three participants will be treated. The decision to escalate, maintain, or de-escalate the dosage will be guided by the TITE-BOIN design. This approach allows for dynamic decision-making by not requiring all pending participants to be DLT evaluable before making dosage adjustments. Exemplary TITE-BOIN decisions are represented in Table 19.

TABLE 19
Dose Escalation/De-escalation Rules for the TITE-BOIN Design

| #Pts | #DLTs | #Pending | Escalate | Stay | De-escalate |
|---|---|---|---|---|---|
| 3 | 0 | ≤1 | Y | | |
| 3 | 0 | ≥2 | Suspend | | |
| 3 | 1 | 0 | | Y | |
| 3 | 1 | 1 | | >0.88 | ≤0.88 |
| 3 | 1 | ≥2 | Suspend | | |
| 3 | 2 | ≤1 | | | Y |
| 3 | 3 | 0 | | | Y &Elim |
| 6 | 0 | ≤3 | Y | | |
| 6 | 0 | ≥4 | Suspend | | |
| 6 | 1 | ≤1 | Y | | |
| 6 | 1 | 2 | ≥0.60 | <0.60 | |
| 6 | 1 | 3 | ≥1.96 | <1.96 | |
| 6 | 1 | ≥4 | Suspend | | |
| 6 | 2 | 0 | | Y | |
| 6 | 2 | 1 | | >0.73 | ≤0.73 |
| 6 | 2 | 2 | | >1.80 | ≤1.80 |
| 6 | 2 | 3 | | >2.87 | ≤2.87 |
| 6 | 2 | ≥4 | Suspend | | |
| 6 | 3 | ≤3 | | | Y |
| 6 | ≥4 | ≤2 | | | Y&Elim |
| 9 | 0 | ≤4 | Y | | |
| 9 | 0 | ≥5 | Suspend | | |
| 9 | 1 | ≤4 | Y | | |
| 9 | 1 | ≥5 | Suspend | | |
| 9 | 2 | 0 | Y | | |
| 9 | 2 | 1 | ≥0.59 | <0.59 | |
| 9 | 2 | 2 | ≥1.65 | <1.65 | |
| 9 | 2 | 3 | ≥2.71 | <2.71 | |
| 9 | 2 | 4 | ≥3.77 | <3.77 | |
| 9 | 2 | ≥5 | Suspend | | |
| 9 | 3 | 0 | | Y | |
| 9 | 3 | 1 | | >0.58 | ≤0.58 |
| 9 | 3 | 2 | | >1.65 | ≤1.65 |
| 9 | 3 | 3 | | >2.72 | ≤2.72 |
| 9 | 3 | 4 | | >3.79 | ≤3.79 |
| 9 | 3 | ≥5 | Suspend | | |
| 9 | 4 | ≤5 | | | Y |
| 9 | ≥5 | ≤4 | | | Y&Elim |

In Table 19: #, number; DLT, Dose Limiting Toxicity; Elim, eliminate; No., number; Pts, participants; STFT, standardised total follow-up time; TITE-BOIN, Time-to-Event Bayesian Optimal Interval Design; Y, yes. Note: “#Pts” is the total number of evaluable participants treated at the current dose level, “#DLTs” is the number of participants who experienced DLT at the current dose level, “#Pending” denotes the number of participants whose DLT data are pending at the current dose level, “STFT” is the standardised total follow-up time for the participants with data pending, defined as the total follow-up time for the participants with data pending divided by the length of the DLT assessment window. “Y” represents “Yes”, and “Y&Elim” represents “Yes and Eliminate”. When a dose is eliminated, all higher doses should also be eliminated.

Exemplary DLTs are shown in Table 20.

TABLE 20
Dose Limiting Toxicities

| Hematologic | Non-Hematologic |
|---|---|
| Grade 4 neutropenia | ALT or AST >3 × upper limit of normal |
| lasting >7 days. | (ULN) and concomitant total bilirubin >2 × |
| | ULN (except for participants with liver |
| | metastases or hepatic comorbidities, e.g., |
| | Gilbert's syndrome) |
| Any febrile neutropenia | AST or ALT >8 × ULN of any duration or |
| (i.e., ≥Grade 3) | AST or ALT >5 × ULN for ≥14 days (all |
| | participants: with or without liver |
| | metastases) |
| Grade 4 thrombocytopenia | Any Grade ≥3 non-haematologic toxicity, |
| | except: Isolated, asymptomatic elevations of |
| | biochemical laboratory values, including |
| | electrolyte abnormalities, that are suspected |
| | to be spurious or respond to medical |
| | intervention within 72 hours. |
| | Nausea, vomiting, or diarrhoea controlled by |
| | anti-emetic or antidiarrheal therapy within |
| | 72 hours. |
| | Transient fatigue that improves within |
| | 72 hours |
| Grade 3 thrombocytopenia | Any death not clearly due to the underlying |
| with bleeding event | disease or extraneous causes |
| Grade 4 anaemia | Any AE that requires a dose delay of >42 |
| | days |
| | Any other clinically significant and/or |
| | unacceptable toxicity judged to be a DLT |
| | by the Investigator and Sponsor |

### Parallel Cohort Evaluation to Optimise Cycle Duration.

Cohorts to assess cycle optimization may be opened in parallel to cohorts assessing higher ²¹²Pb dose levels. However, it is not permitted to increase the ²¹²Pb Dose level and decrease cycle duration in the same Dose Escalation Cohort. Example nomenclature for Cohorts is provided in Table 21.

TABLE 21
Dose Escalation Cohorts

| Cohort | Dose Level (DL) | Schedule |
|---|---|---|
| 1 | DL 1: 100 MBq | 6-week cycle |
| 1S* | DL 1: 100 MBq | Cycle length optimization: e.g. |
| | | 4-week cycle (+/−1 week) |
| 2 | DL 2: 150 MBq | 6-week cycle |
| 2S* | DL 2: 150 MBq | Cycle length optimization: e.g. |
| | | 4-week cycle (+/−1 week) |
| 3 | DL 3: 200 MBq | 6-week cycle |
| 3S* | DL 3: 200 MBq | Cycle length optimization: e.g. |
| | | 4-week cycle (+/−1 week) |
| X | Other dose level X | 6-week cycle |
| | (e.g. 75 MBq) | |
| XS* | Other dose level X | Cycle length optimization: e.g. |
| | (e.g. 75 MBq) | 4-week cycle (+/−1 week) |

Cohorts with shorter cycle durations may follow the same Dose Escalation procedure guided by the TITE-BOIN design (assumptions as for the 6-week cycle schedule), but with the additional precaution that Dose Escalation will only be permitted to a Dose Level already ‘cleared’ for a 6-week cycle. The Table 22 below outlines the decisions tree to open parallel cohorts for cycle optimisation based on prior Dose Level clearance.

TABLE 22
Decision Table for Permitting Opening Cohorts for Cycle Length
Optimisation in Parallel During ²¹²Pb Dose Level Escalation

| | Dose Escalation | Dose Escalation | Initiate parallel |
|---|---|---|---|
| | cleared for 6- | cleared for shorter | Cohort with |
| Dose | week cycle at | cycle at lower | shorter cycle |
| Level | Dose Level | (−1) Dose Level | at Dose Level |
| 100 MBq | Yes | NA | Yes |
| 100 MBq | No | NA | No |
| 150 MBq | Yes | Yes | Yes |
| 150 MBq | No | Yes | No |
| 150 MBq | Yes | No | No |
| 150 MBq | No | No | No |
| 200 MBq | Yes | Yes | Yes |
| 200 MBq | No | Yes | No |
| 200 MBq | Yes | No | No |
| 200 MBq | No | No | No |

In table 22: NA, not applicable.

### Dose Decisions

Safety data will be reviewed on an ongoing basis during the study for all participants and will be regularly discussed with the Investigators. At the time of a potential cohort Dose Decision, indicated by the TITE-BOIN (e.g., Table 19), all available clinical safety, tolerability, PK, and tumour response data will be discussed and evaluated.

Dose Decisions will be made jointly by the Sponsor and Investigators based on the totality of the data from all preceding Dose Escalation cohorts, and may include: dose escalation to the next Dose Level, dose de-escalation to a lower Dose Level, further recruitment at the same Dose Level, adjustment of the dosing schedule (e.g., cycle duration based on toxicity incidence and rate of recovery), and/or termination of Dose Escalation.

Dose Decisions will be guided by the TITE-BOIN design according to prespecified rules based on actual observations of TEAEs that fulfil the DLT criteria, and observed preliminary antitumour activity (see e.g., Table 19).

Evaluation of Antitumour Activity: During Dose Escalation, once a cohort with a given Dose Level/Schedule is ‘cleared’ by DLT evaluation, the cohort may be backfilled to up to nine evaluable participants if a primary efficacy signal is observed. This approach allows for the gathering of antitumour activity data (e.g., radiological and PSA50 responses), which will be used, along with the clinical safety data and available PK and biodistribution data, to select the Recommended Dose and Schedule for Dose Expansion. The safety data of these additional participants will be discussed by the Investigators, the Sponsor, and the CRO. Any DLTs or Regimen Impacting Toxicities (RITs) will be taken into consideration for subsequent Dose Escalation Decisions.

### End of Dose Escalation:

Dose Escalation will end if one of the stopping criteria is met:
The recommended dose for Expansion has been determined.
The Maximum Tolerated Dose (MTD) is determined (or the maximum planned dose is tolerable).
The minimum dose tested is toxic.
At any time where there are already nine or more evaluable participants treated at the ²¹²Pb dose level recommended for the next Dose Escalation cohort.

The Dose Escalation part of the study will progress into the Dose Expansion part. Based on available data, participants in the Dose Escalation Cohort at the Recommended Dose and Schedule for Expansion may be included in Dose Expansion Group A..

### Example 11: Dose Expansion

The Dose Expansion part of the study will progress seamlessly from Dose Escalation. The Dose Expansion part of the study aims to determine the preliminary antitumor activity and safety of ²¹²Pb-Compound 1 and confirm the recommended dose and schedule for further development. Participants in Dose Expansion will be monitored for adverse events (for example, DLTs) and antitumor activity to allow continual monitoring of the recommended dose and schedule throughout the clinical study.

There will be 2 separate Groups in Dose Expansion of 30 participants, with indication-based populations of mCRPC specified based on participant's prior anticancer therapies, namely:
Group A: patients who have not received prior treatment with ¹⁷⁷Lu-PSMA
Group B: patients who have been treated with ¹⁷⁷Lu-PSMA.

Participants in Group A will receive the recommended dose and schedule of ²¹²Pb-Compound 1 from Dose Escalation. For Group B, a safety run-in cohort of at least 3 evaluable participants will first be opened at one dose level lower than the recommended dose and Schedule from Escalation. Clearance of the safety run-in cohort by Dose Decision Meeting (using the same process as described for Dose Escalation) must occur before Group B may proceed to open at the recommended dose and schedule.

More than one dose level or schedule of ²¹²Pb-Compound 1 may be selected to be tested in any Dose Expansion Group, either sequentially or concurrently, based on the benefit-risk profile observed during Dose Escalation. Randomization will be performed if more than one dose level/schedule is tested concurrently in same Dose Expansion group.

The Dose Expansion part of the study will be conducted in an adaptive two-stage design with one interim assessment in each Group. The expected number of evaluable participants in each group is 30. An interim futility assessment will be performed after approximately 50% of the intended total number of evaluable participants in each group have completed the tumor assessments indicated before the end of Cycle 3.

### Example 12: Phase 1/2 Study Population

### Inclusion Criteria

Participants are eligible to be included in the study only if all the following criteria apply:
Male participants at least 18 years of age at the time of the signing informed consent.
Eastern Cooperative Oncology Group performance status (ECOG PS) of 0 or 1.
Life expectancy of at least 6 months.
Metastatic castration-resistant prostate cancer (mCRPC) with histological confirmation of prostate adenocarcinoma. The mCRPC may be progressive.
Availability of fresh or archival tumor samples obtained on or after the most recent anticancer therapy. Tumor samples obtained before the last anticancer treatment may be accepted on agreement between the Investigator and Sponsor. Availability of alternative profiling of tumour mutational status may be accepted instead on agreement between the Investigator and Sponsor.
Previous treatment with at least one novel androgen receptor axis targeted agent (ARATA).
Prior orchiectomy and/or ongoing androgen-deprivation therapy and a castrate level of serum testosterone (<50 ng/dL or <1.7 nmol/L).
Prior treatment with at least 1 but no more than 2 taxane regimens or been deemed ineligible for or refused taxane therapy on consultation with their physician.
Dose Expansion Group B only: prior treatment with at least one cycle of ¹⁷⁷Lu PSMA therapy greater than 6 weeks from start of study treatment.
At least one PSMA-avid distant metastatic lesion confirmed by PSMA-PET/CT with a permitted PSMA-targeting imaging agent. A PSMA-avid distant metastatic lesion is defined as physiological uptake greater than that of adjacent tissue in lesions of any size in any organ system. The participant should have no PSMA negative lesions, defined as physiological uptake equal to or lower than that of adjacent tissue in any lymph node with a short axis of at least 2.5 cm, in any solid organ lesion with a short axis of at least 1.0 cm, or in any bone lesion with a soft-tissue component of at least 1.0 cm in the short axis, confirmed by PSMA-PET/CT with a permitted PSMA-targeting imaging agent within 28 days before start of study treatment.
Dose Expansion Groups only: At least one measurable lesion per RECIST 1.1 criteria.
Adequate bone marrow, renal and hepatic function, as assessed by the following laboratory requirements within 28 days before start of study treatment:
   a. Absolute neutrophil count (ANC)≥1.5×10⁹′L
   b. Hemoglobin ≥9.0 g/dL
   c. Platelet count ≥100×10⁹/L
   d. Serum creatinine ≤1.5×ULN and estimated glomerular filtration rate (eGFR)≥60 mL/min/1.73 m², according to the Modified Diet in Renal Disease (MDRD) abbreviated formula.
   e. Total bilirubin ≤1.5× the upper limit of normal (ULN) (except if confirmed history of Gilbert's disease, in which case direct bilirubin must be normal)
   f. ALT and AST<2.5×ULN (≤5×ULN for participants with liver involvement)
Male participants must agree to use highly effective contraception during treatment and for 6 months after the last study treatment and refrain from donating sperm during this period.
Contraceptive use should be consistent with local regulations regarding methods of contraception for those participating in clinical studies.
Capable of giving signed informed consent which includes compliance with the requirements and restrictions listed in the Informed Consent Form (ICF) and in this protocol.

### Exclusion Criteria

Participants are excluded from the study if any of the following criteria apply:
Urinary obstruction, or where there is deemed a substantial risk of urinary obstruction due to pelvic disease.
Untreated CNS metastases (therapy includes surgery, radiotherapy, gamma knife) or treated CNS metastases which are not adequately controlled. Treated CNS metastases are permitted provided thy are neurologically stable, asymptomatic, and corticosteroids are not required for to maintain neurologic integrity.
Symptomatic medullary cord compression, or clinical or radiologic findings indicative of impending cord compression.
Diffuse bone or bone-marrow involvement i.e., a “superscan”: defined as bone scintigraphy in which there is diffuse and intense activity throughout the axial skeleton due to metastatic disease. Participants with borderline/ambiguous assessment may be included on a case-by-case basis on agreement with the Investigator and Sponsor.
Participants known to be affected by genetic defects linked to radiation hypersensitivity.
History of Myelodysplastic syndrome (MDS), treatment-related acute myeloid leukemia (t-AML) or features suggestive of MDS/AML.
A known additional malignancy that has required active treatment within the past 2 years before start of study treatment, except for adequately treated basal or squamous cell carcinoma of the skin, or carcinoma in situ that has undergone curative therapy.
Infections of CTCAE v5.0 Grade 2 not responding to therapy or active clinically serious infections of CTCAE Grade >2.
Human immunodeficiency virus (HIV) infection with any of the following: a CD4+ T-cell count of <350 cells/uL; a history of AIDS defining opportunistic infection within the past 12 months; established on stable antiretroviral therapy for less than 4 weeks; an HIV viral RNA level >400 copies/mL; requiring either antiretroviral therapy prophylactic antimicrobials expected to cause significant drug-drug interactions or overlapping toxicities.
Active hepatitis B virus (HBV) or hepatitis C virus (HCV) infection requiring treatment. Participants with chronic HBV or HCV infection are eligible provided the infection has been controlled with appropriate treatment and the viral load is below the limit of quantification.
Serious, infected non-healing wound, ulcer, or bone fracture.
Major surgery, open biopsy, or significant trauma within 4 weeks before start of study treatment.
Impaired cardiac function or clinically significant cardiac disease, including: congestive heart failure (CHF) (New York Heart Association (NYHA) Class II, III or IV); unstable angina, new-onset angina (within 3 months of start of study treatment), myocardial infarction less than 6 months before the start of study treatment; clinically significant arrhythmia; uncontrolled hypertension (systolic blood pressure >140 mmHg and/or diastolic blood pressure >100 mmHg).
Known allergies, hypersensitivity, or intolerance to the study treatment (including excipients).
Prior systemic anticancer therapy (e.g., chemotherapy, immunotherapy or biological therapy (including monoclonal antibodies)) or investigational therapy within 4 weeks before the start of study treatment, with the exception of Luteinizing Hormone-releasing Hormone (LHRH) or Gonadotropin-releasing Hormone (GnRH). Start of study treatment is allowed in shorter timeframes provided 5 half-lives of the prior agents(s) have elapsed.
Previous PSMA-targeted radiopharmaceutical treatment except:
   a. Participants who received only a micro-dose of ²¹²Pb-Compound 1 in a Phase 0 study.
   b. Participants in Dose Expansion Group B must have had prior treatment with ¹⁷⁷Lu-PSMA; this must have ended at least 6 weeks before the start of study treatment.
Prior treatment with any other systemic radiopharmaceutical therapy within 6 months before the start of study treatment (e.g., agents based on ¹⁷⁷Lu, ²²⁵Ac, ²²³Ra, etc.).
Prior definitive radiotherapy completed less than 6 weeks before the start of study treatment; palliative radiotherapy within this time is allowed provided (i) no more than 10% of the participants' bone marrow is irradiated, and (ii) it does not encompass all potential target/measurable lesions for participants in dose expansion.
Prior external beam radiotherapy (EBRT) where dose to a critical organ (e.g., kidneys, heart, lung) has exceeded the EBRT normal organ tolerance dose limit.
Hemi-body irradiation within 6 months of the start of study treatment.
Previous high-dose chemotherapy needing hematopoietic-stem-cell-rescue.
Previous autologous or allogeneic stem-cell transplantation.
Ongoing toxicity CTCAE Grade >2 due to prior anti-cancer therapy that is significant or not stabilized (including Grade >2 dry mouth [i.e. xerostomia]). Chronic toxic effects Grade <2 where no further resolution is expected do not require exclusion (e.g., chemotherapy-induced neuropathy, fatigue, alopecia, anorexia, etc.).
Live vaccines within 4 weeks before the start of study treatment; vaccines for COVID-19 are allowed.
Biological response modifiers, such as granulocyte colony-stimulating factor (G-CSF), granulocyte macrophage colony-stimulating factor (GM-CSF) and erythropoietin within 3 weeks before start of study treatment. Chronic use of erythropoietin is allowed, provided there is no dose modification within 3 weeks before the start of study treatment.
Systemic corticosteroids >10 mg of prednisone/prednisolone per day or herbal products that may decrease PSA levels (e.g., saw palmetto) within 4 weeks before the start of study treatment.

### Example 13: Experimental Metastasis Models of Prostate Cancer

Experimental metastasis models of prostate cancer involve injecting prostate cancer cells directly into the blood stream of a model animal, such as mice, to study the ability of cancer cells to grow into distant organs. Intravenous, intracardiac, intrailiac artery, intrasplenic, intratibial, intraperitoneal, or intra-bone marrow injection of prostate cancer cells may lead to establishment of metastasis in distant organs. Exemplary prostate cancer cells used in experimental metastasis models include LNCaP, PC3 (including PSMA transfected PC3-PIP cells), DU145, VCaP, MDA PCa 2b, LAPC4, and 22Rv1. Intracardiac injection allows for dissemination of cancer cells and is commonly used to model bone, liver, lung, and lymph node metastasis. As an example, metastatic prostate cancer may be established in mice via intracardiac injection of 10⁶-10⁷ prostate cancer cells, such as PC3 or PC3-PIP. After some time, the mice are intravenously injected with a ²¹²Pb radiopharmaceutical conjugate disclosed herein, such as ²¹²Pb-Compound 1, and sacrificed up to 150 days later. It is anticipated that untreated mice will develop distant organ metastases in bone, viscera (e.g., lungs, liver, heart, etc.), lymph node(s), or a combination thereof. The location of metastatic lesions may be detected using any appropriate method known in the art, such as bioluminescent imaging. It is expected that treatment with the ²¹²Pb radiopharmaceutical conjugate disclosed herein, such as ²¹²Pb-Compound 1, will prevent or reduce the formation of distant metastases, and in particular prevent or reduce bone lesions. Further, it is expected that the ²¹²Pb radiopharmaceutical conjugate disclosed herein, such as ²¹²Pb-Compound 1, will prevent or reduce the formation of said metastases to a greater extent than non-²¹²Pb radiopharmaceuticals, such as ¹⁷⁷Lu-PSMA-617.

We claim:

1. A method of treating a prostate cancer in a human subject in need thereof, comprising administering to the human subject a radiopharmaceutical conjugate, wherein the radiopharmaceutical conjugate comprises 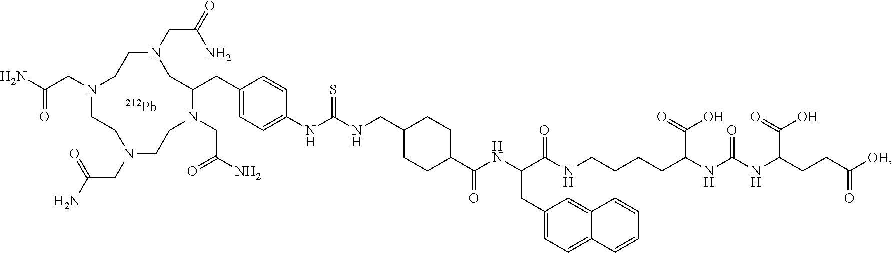
or a pharmaceutically acceptable salt thereof,
wherein the radiopharmaceutical conjugate is administered to the subject about once every week, about once every 2 weeks, about once every 3 weeks, about once every 4 weeks, about once every 5 weeks, or about once every 6 weeks.

2. The method of claim 1, wherein the radiopharmaceutical conjugate is administered to the subject about once every 3 weeks.

3. The method of claim 1, wherein the radiopharmaceutical conjugate is administered to the subject about once every 4 weeks.

4. The method of claim 1, wherein the radiopharmaceutical conjugate is administered to the subject about once every 5 weeks.

5. The method of claim 1, wherein the radiopharmaceutical conjugate is administered 2 times (i.e., for 2 cycles), 3 times, (i.e., for 3 cycles), 4 times, (i.e., 4 cycles), 5 times (i.e., 5 cycles), 6 times (i.e., 6 cycles), 7 times (i.e., 7 cycles), 8 times (i.e., 8 cycles), 9 times (i.e., 9 cycles), 10 times (i.e., 10 cycles), 11 times (i.e., 11 cycles), or 12 times (i.e., 12 cycles).

6. The method of claim 1, wherein the radiopharmaceutical conjugate is administered 4 times, 5 times, or 6 times.

7. The method of claim 1, wherein the radiopharmaceutical conjugate is administered in an amount between 50 kBq to 500 MBq per dose.

8. The method of claim 1, wherein the radiopharmaceutical conjugate is administered in an amount of about 75 MBq per dose to about 300 MBq per dose.

9. A method of preventing or reducing the formation of a metastatic bone lesion in a human subject in need thereof, comprising administering to the subject a radiopharmaceutical composition comprising
(a) a compound of Formula (I):
TL-L-R^{M} Formula (I)
or a pharmaceutically acceptable salt thereof, wherein:
TL is a PSMA targeting ligand comprising a urea;
L is a bivalent linking moiety; and
R^{M} is a metal chelator; and
(b) a radionuclide bound to the metal chelator, wherein the radionuclide is ²¹²Pb;
wherein the human subject has prostate cancer; and
wherein the radiopharmaceutical conjugate is administered to the human subject in an amount between 50 kBq to 500 MBq per dose.

10. The method of claim 9, wherein the radiopharmaceutical conjugate comprises a structure of Formula (I-B-1) or Formula (I-B-2): 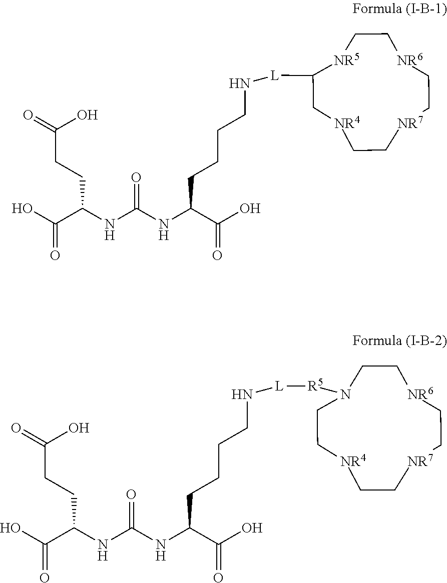
or a pharmaceutically acceptable salt thereof, wherein:
R⁴, R⁵, R⁶, and R⁷ are each independently selected from the group consisting of —C₁₋₃alkylene-C(═O)OR³, and —C₁₋₃alkylene-C(═O)N(R³)₂, wherein each C₁₋₃alkylene is optionally substituted with —C(═O)OH; and
each R³ is independently selected from the group consisting of H and C₁₋₆alkyl;
provided that R⁵ in Formula (I-B-2) is —C₁₋₃alkylene-C(═O)—, wherein the C₁₋₃alkylene is optionally substituted with —C(═O)OH.

11. The method of claim 9, wherein the compound of Formula (I) is 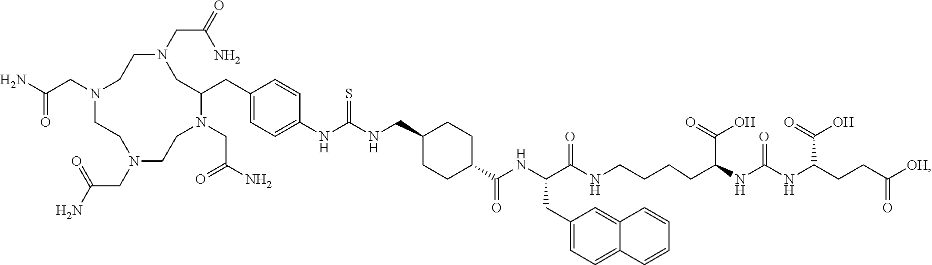
or a pharmaceutically acceptable salt thereof.

12. The method of claim 9, wherein the compound of Formula (I) is 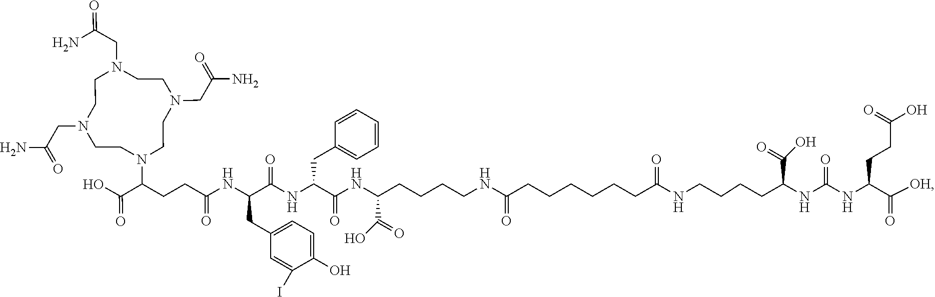 [SMILES: NC(=O)CN1CCN(CC(N)=O)CCN(C(CCC(=O)N[C@H](Cc2ccc(O)c(I)c2)C(=O)N[C@H](Cc2ccccc2)C(=O)N[C@H](CCCCNC(=O)CCCCCCC(=O)NCCCC[C@H](NC(=O)N[C@@H](CCC(=O)O)C(=O)O)C(=O)O)C(=O)O)C(=O)O)CCN(CC(N)=O)CC1]
or a pharmaceutically acceptable salt thereof.

13. The method of claim 9, wherein the prostate cancer is local and administering the radiopharmaceutical conjugate to the subject prevents or reduces skeletal metastases.

14. The method of claim 9, wherein the prostate cancer is metastatic, but does not comprise bone lesions, and administering the radiopharmaceutical conjugate to the subject prevents or reduces further skeletal metastases.

15. The method of claim 9, wherein the radiopharmaceutical conjugate is administered in an amount of about 75 MBq per dose to about 300 MBq per dose.

16. The method of claim 9, wherein the radiopharmaceutical conjugate is administered in an amount of about 75 MBq per dose, about 100 MBq per dose, about 150 MBq per dose, about 200 MBq per dose, about 250 MBq per dose, or about 300 MBq per dose.

17. The method of claim 9, wherein the radiopharmaceutical conjugate is administered about once every week, about once every 2 weeks, about once every 3 weeks, about once every 4 weeks, about once every 5 weeks, or about once every 6 weeks.

18. The method of claim 9, wherein the radiopharmaceutical conjugate is administered 2 times (i.e., for 2 cycles), 3 times, (i.e., for 3 cycles), 4 times, (i.e., 4 cycles), 5 times (i.e., 5 cycles), 6 times (i.e., 6 cycles), 7 times (i.e., 7 cycles), 8 times (i.e., 8 cycles), 9 times (i.e., 9 cycles), 10 times (i.e., 10 cycles), 11 times (i.e., 11 cycles), or 12 times (i.e., 12 cycles).

19. A method of treating a prostate cancer in a human subject in need thereof, comprising:
administering to the human subject a radiopharmaceutical conjugate, wherein the radiopharmaceutical conjugate comprises,
(a) a compound of Formula (I):
TL-L-R^{M} Formula (I)
or a pharmaceutically acceptable salt thereof, wherein:
TL is a PSMA targeting ligand comprising a urea;
L is a bivalent linking moiety; and
R^{M} is a metal chelator; and
(b) a radionuclide bound to the metal chelator, wherein the radionuclide is ²¹²Pb; and
wherein the radiopharmaceutical conjugate is administered in combination with folate.

20. The method of claim 19, wherein the compound has the structure of 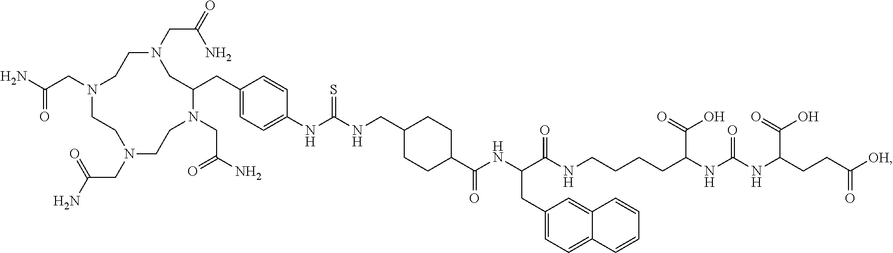 [SMILES: NC(=O)CN1CCN(CC(N)=O)CCN(CC(N)=O)C(Cc2ccc(NC(=S)NCC3CCC(C(=O)NC(Cc4ccc5ccccc5c4)C(=O)NCCCCC(NC(=O)NC(CCC(=O)O)C(=O)O)C(=O)O)CC3)cc2)CN(CC(N)=O)CC1]
or a pharmaceutically acceptable salt thereof.

21. The method of claim 19, wherein the compound has the structure of 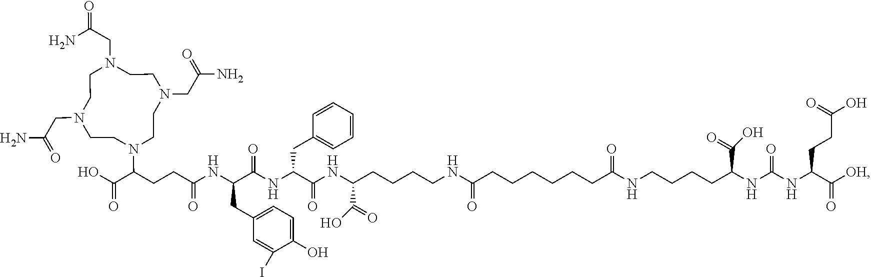 [SMILES: NC(=O)CN1CCN(CC(N)=O)CCN(C(CCC(=O)N[C@H](Cc2ccc(O)c(I)c2)C(=O)N[C@H](Cc2ccccc2)C(=O)N[C@H](CCCCNC(=O)CCCCCCC(=O)NCCCC[C@H](NC(=O)N[C@@H](CCC(=O)O)C(=O)O)C(=O)O)C(=O)O)C(=O)O)CCN(CC(N)=O)CC1]
or a pharmaceutically acceptable salt thereof.

22. The method of claim 19, wherein the folate is administered prior to, or concurrently with, the radiopharmaceutical conjugate.

23. The method of claim 19, wherein the folate is administered concurrently with the radiopharmaceutical conjugate.

24. The method of claim 19, wherein the folate is administered up to 12 hours prior to administering the radiopharmaceutical conjugate.

25. The method of claim 19, wherein the folate is administered in an amount between 100 μg and 25 mg.

26. The method of claim 19, wherein the folate is folic acid, dihydrofolate (DHF), tetrahydrofolate (THF), 5, 10-methylenetetrahydrofolate (5, 10-MTHF), or 5-methyltetrahydrofolate (5-MTHF).

27. The method of claim 19, wherein the radiopharmaceutical conjugate is administered in an amount between 50 kBq to 500 MBq per dose.

28. The method of claim 19, wherein the radiopharmaceutical conjugate is administered in an amount of about 75 MBq per dose to about 300 MBq per dose.

29. The method of claim 19, wherein the radiopharmaceutical conjugate is administered about once every week, about once every 2 weeks, about once every 3 weeks, about once every 4 weeks, about once every 5 weeks, or about once every 6 weeks.

30. The method of claim 19, wherein the radiopharmaceutical conjugate is administered 2 times (i.e., for 2 cycles), 3 times, (i.e., for 3 cycles), 4 times, (i.e., 4 cycles), 5 times (i.e., 5 cycles), 6 times (i.e., 6 cycles), 7 times (i.e., 7 cycles), 8 times (i.e., 8 cycles), 9 times (i.e., 9 cycles), 10 times (i.e., 10 cycles), 11 times (i.e., 11 cycles), or 12 times (i.e., 12 cycles).
